SEQUENCE LISTING (63 sequences)

SEQ ID NO: 1 - DNA, 855 bp, Pantoea ananatis
  source 1..855: mol_type = other DNA
atgatcaaaaaacttattgcagaaaagggtactctgatttttattgaggcccataatccgctctccgcattaattgcgtctaaagcagaacaaactaattcagaaggccgtattgtcaaatttgacggtatatggtcaagctcgttaacggactcagcatctcgcggtattcccgataacgaaacactggcattaagcagcaggttagaaaatattgctgatatccgaaatgtgacagacatgcccatcatcatggatgctgatacggggggaaaaccagaacattttagttattacgtaaaaagaatgattaacaacggtgtaaatggcgtcatcatcgaagataaaacaggattaaagaaaaattctttgttcggcactgaagtagaacagactctcgcagatattaatgatttttcagagaagattaaaagaggaaaatctgcagtttatattgatgattttatgatcatagccagacttgaaagtcttattgcagggttcgacgtagaacatgcactcgaacgtgccgacgcatacgtcgaagccggggcagacggaattatgattcatagttgtaagaagactccggatgaggttttcttattcagtacgaaatttcggaaaaaatatccatcagtaccattaatttgtgttcctactacttattctgcaaccagcaacagagaactcagtgaagcgggttttaacgtgatcatttatgcaaaccatatgctcagggctgcttataaagcaatggaaaatgtttcaaaagaaatattgagatatggcaggacggcagagatagaaaaatcttgcatgagtgtaaaggaaattatttcactgattccttaa

SEQ ID NO: 2 - DNA, 1380 bp, Pantoea ananatis
  source 1..1380: mol_type = other DNA
ttgcatgagtgtaaaggaaattatttcactgattccttaaagagaatgaccatgaaaaaaaaagagatgataataggtgcctatatatcgtatggaacaggacatcatcctgcttcatggcgcgaaagtggcgtaaatgcagcggcagcgcttgatattgatacgtatgccaatcttgcaagagtatgtgagaaaggattagctgatttactttttcttgcagatacgccttctgtatttcaggacaatatggacggttatggcagcagggtatctgtactggagccattatcattattatcgtatttagcatctcaaacacaaaatattgggctggttgcgacggcttcaacgacgtacaaacacccttacaacattgcgagggaatttgcttcgctggattacattagcaaaggaagggcagggtggaatctggtgacatcctcaaagtcggatgcggctaaaaactttggccttgccgctcatccagaacattcaaagcgttatgatatggcctgggaagcatggcaggttatcagtggtttgtgggacagctgggaagacaatgctttagtaaggaataaaaccagtggacaattcttcgttaaggataaataccgggaaataaattttgagggtgaatattttaatgtaaaaggcccattaaacatagctcgccctcctcagggctatcccgtcatcgtccaggcgggttcctctgaggaaggaaaagagttagctgcaaaaacagcagatattgtttttactgcacaaaacaatattgaagatgcaaaaaaattctatgatgatcttaaagggcgaatggaaaaatacggaaggtcaaagagtgaacttcttattcttcccggattaagcttttatattgcaagtgacgaatctaaagcccgtaaaaagcttaatgatctcaacgcactgatccctcaatcatttggcttaagtatgttatcagatttactgggtggggttgatttaaaaaacaatgatcctgaaggaccattaccagatttaccgaaatctaatggtaatcagagtagacaaaaaattattatcgatttagctcgaaaagaaaaactatctatcaagcaactttatgaaaaaataatcatttcaagaggacattatacatttacaggttcctatcaagatttagcagatgagatgattaagtgggttgaaaatgaagcatgtgatggtttcaacattatgcctcctctcatgcctgaatctcttattaatcttttcgatcatgtcattccacttattcaggcaagaggatggtataaaaaatcatattctactggaacattaagagaaaaactggggcttaaaagacctactaataaattgtttaatcaataa

SEQ ID NO: 3 - DNA, 1122 bp, Pantoea ananatis
  source 1..1122: mol_type = other DNA
atgcttaataaaaatctgattcttgaagataccactttacgtgatggtgagcaggcgccaggtgttgcatttacaccagagcaaaaagtagaaattttttatctacttgcaaatatgggcgttaaatggatcgaagccggaatacctgcgatgaagggtgatgaagtaaaggctctgtcggaaatgttagagagaaaaaatgaaattaacatcatcgcgtggaaccgaggcgtgcttgaagacattgagtacagtatctcacttggattcaaagcggtgcatatcgggctaccgacttcagctatccatttagagaaaagcgttaagaaagataagtcctggcttgtaaagacggcttcagatttagttaagtttgccaaagacaaagggatgtttgtttctatcagcgcagaagatataggccgaacagatattggattcttacaggagtatgcacaggtagttgctgaggctggagccgatcgtcttcgcctctctgacacgattggtattctttctcctgcacaatacaaagaaaaagtctctttgttaaataagaacgtcaacatcgatttgcagtgccattgccacaatgattttggttttgcagttgctaacacgctggcaggcattgaagcaggagcacgctactttcatgtctgcgtcaatggcattggtgaaagggctggaatgccagacctggcacaagttgctatggcattgcactttttccacggggttgatttagggctcgatttaacaaaattaatcgcgttgagtgaaacggtcgccaggtacagccatcaaaaaatcagtccatggcagccgatcgtaggcgataacgtttttgcacatgaatcgggcattcacgcaaatggtatgctcaaagacagcagtacttttgaacccttcgacccagctacggtgggaggagaacgacgtctggtcgtgggtaaacattccggtcgcgccattatcaaacattttctcgaagaatcaggcgtgaaagctgccgacgataaggctcttgatcgctgtttagaacgcgtgagaagtcatgccgtgcgccaccccggtgggatccctccacatgtattagttgatctgtataccgcggggtaa

SEQ ID NO: 4 - DNA, 1239 bp, Pantoea ananatis
  source 1..1239: mol_type = other DNA
atgaaaagtaatcagccgattgttaaccagatcattgcgtcacacagtggcagaggtcaggtttcagcaggtgaactgatcacggtagatgttgactacgtctatgttcaggatggaaattcaccgaccgtggcaaaactgtttcaggattatcatctgtctgaggtgctaaaacccgataaaatcgggttcttcttcgaccattcagttctggtacctgataaaaccatggctaaacgtgtcaacgaggccatggaatttgcaaaaaaacttggaataaacatctattcacgaggggagggaattagtcacgtcattgccctggagagtaaaatatttaaacccggcaatatagtgctgggcgcagattcccatacttgtacagggggggccgtacagtctttagcgctgggaatgggggcttcggatattctggttgctatgttaacaggacaaacgtggttgaaagtccctcaaacagtccatttgtgtattaagggtaaaacgggaaaagatgtgcgggcaaaagatgtcatgttggcacttttaaataagtacggacaaacaccatttctttataaatcgatcgaagtttcaggggaatgggcagaagagctaacgcttgacgaagctgcaagttttgcaagtatggctgttgagttaggagccaaatgcatatttatgccagatgggcaaggcaggcctgaggggctattgaaggcggatgcctcagtggcagacagtgtcatcaatttttctgtatcagaattaatgccccatatcgcaccacctcacagtcctttgtatgctaaacctgcaaatgactttgagggtctgaaatttgattatattttcattggaagctgtactaacagcagacttgaagatatcaaagaggtggccgaaattgttgctggtaaaacaatacatcccgatattcactgccttctgacgccaggatcgaaaagtgtttatctaaaagctctccaggcgggatatatcgatacgcttatccgctcgggcattattgtcaccccaccgggttgtggagcttgtgtgggtacccaaggaaccattcctgcggatggggagaaagtattaagtacgatgaaccgcaattttaagggaagaatggggaatgctgaggcagacatctttttatgttctccacgaactgcagcgatggttgcattgaacggcactgttccacattttgagggagagtccgcatatgagtaa

SEQ ID NO: 5 - DNA, 564 bp, Pantoea ananatis
  source 1..564: mol_type = other DNA
ctgttccacattttgagggagagtccgcatatgagtaaaactgctgaaaattatcgcgtcagacgcgtggaagggaacatctcgaccgacgatattatacctgcgcgctataaacatatgtataccgagccggcccagctggcaccgcatctttttgagagccgttttcccggatttagggaaacgctcagtatcaatgatgtgcttgtatgtgatcaaatattcggtatagggagttcgcgggagcaggcagtaacaactctgttggcatgtggtgttaaatatgtattttccccttctttcgggaggattttttttaggaactcttggaatttgggtttacatgcgattgaggtcgatacgagtgaacttgcagatttaagtgaaattaaaatagaactgactggaggggtaatttatacagaaaataatcaaataaattttttccctcccagctcgcagatgacggcaattgtcagtgcaggtggcataataccctacaccataaataaaattatggaaaaaaaaggtgatattttaagaggttatagcaatgaaaagtga

SEQ ID NO: 6 - DNA, 768 bp, Pantoea ananatis
  source 1..768: mol_type = other DNA
atgaaaagtgaaaagtttgatggtttggctgataactatgataaatatcgtccccgttatcctgcaatccttttcaaggaaatccatgactggatgcagccgtctgccaaaaatatatacgatattggcgcaggcacaggtattgctattgaaggtatgacacgtgtcactggaaaacactatgatttcacggcgatagatatttctgaagatatgataaaaaaaggaagggaaaaactgcctggtacgacttgggttaaaggaaaagcggaagatattctttctgataaaagccgtattgacgtcattatggcggcacagtccttccaatggatggatagagctaaaacattagaagtttcgataaaatctttaaataagggcggggtttttgcagttttgcaaaacaatcgagattacagaaataatgaaatgcttaacaaatatgaaggtttgctagagaaatttagtccaggttacagcagacattatcgcgactatgactatgaaaatgaaatcaccaatgtttttaaattgcctattgctaactttaagaaagtcgtcacagggtggactatggaaatgatttcagaagatttttttggattcatttcttcctcaacccaggtacaaagagctattgagaacgatcgtaatggattctggaaggagattgaaatcttgattgacgaacactcagttggtggaaaaattagcatagattatataagtgagttatttatagctaagaagcgtgatgattcatag

SEQ ID NO: 7 - DNA, 477 bp, Pantoea ananatis
  source 1..477: mol_type = other DNA
atgattcatagcatgacgattcaggacatcagcattgaacaagcttcgtatgaagatgcgaagctgttaagaaaggctttagaaaaagtttacgaaccttatacactgaatttctcacctaccgctttgcagtttactgaaaatatcattgctcaggaatcctcgaagtggctagtcgcgaaatacaagtcagacattgtgggggcagtgagatatgaactttatgatatttatctggacttccattttctctgcgtcacaccaccattcagaaaaatgggagtagggaatgaattatttcataaactgaagaaaatagcgtatgaaaaaagaaaggattttatgaagatcgttttgcgagattcgctaagctataaccgacgctattttgaaagtaaaggattttacttttatcataaataccagacaaatatgcatagtgtatttattttaaaattaaacggtgaaaagccatga

SEQ ID NO: 8 - DNA, 1104 bp, Pantoea ananatis
  source 1..1104: mol_type = other DNA
atgaataaaaaagcattggttattgggttgaaaagtaatatggaaagagtcatcaaagggctcaatgagatagaatttattattattgacagaggaacactggataacgaaagtatagattatatcattaatctttcagatgatttaatgcataaaaaatcattcagttatgttatagccagttctgaggattttatcgctttggctgggttattgcgtaatcgttattcactttatggtgaaaaatattataaaagtacaattgcaaccaataaattcctgatgcgtaatttttgctcaggttttttatcctgtccaaagttctggctatcaggtgagatcatcaattcagagaatcttttactgtcatcccagaaagattacatcgtaaaacctcttacaggtagttctgcaaaacatgtcgaaacagtcacacaggaagacttaaaccaatatcttaatgaaaataacaaattgatgttaattgaagaaaaggttttaatgagagatgaatatcatcttgattgtataatcaaagacggtaatatcctcttttcaacactgtcaatttacgacaggcctatactcgaggcaaaaagtaaaaacagagccagtatcaacttacccgatggcacacggcttcatgaagatgccttaatacttgccacgaatttgcaaagtcattttgaaatgactaatggcgtttttcatattgaaatgtatcatactcaggatggatttatactcggtgagtttggtatccgccctcccggagcgggtgtgaccgatatgtattatatgtatagaggcgtcgatttctgggaggcatttatttattctcagattgataaagaatttattttacccctaaatcataagtccgataaatattgtgctgctattgggatatgttcttctttcccggtcgatgatatcagaagtacgtcaaaagtatccgtggataaatatgtaaatttgcgtgagaatgctgctaaacccgcagttcccagttcaacgtcttttaatcatatgatttatgtttcttcatcatccttagaagaaataaggaattttttgcatgatatttctgacagacaagagtaa

SEQ ID NO: 9 - DNA, 1239 bp, Pantoea ananatis
  source 1..1239: mol_type = other DNA
atgttaaaatatgaaaccaaaaaaaacgctttcttccagatatacaattttctttcatgctcggctgaagggattcagacggttatatttttgtggcttatatatcatgaaactcatagtccgatgcttgtcagtctgactatagtttcttcatatcttccatcagccgtattaggattctttttcctcaaaaaagctgatgcgagcagtcctggaaaacagttatttatcagtaatgtttctcttagtgccatatcgctaattgtctattttattttaatgagaaatgagggttttgaactaatcacgctcagtatcttttatttggcacaggccgtattatctgttgtcaaaatgtttaataagacatcccaaaatcgcattatcagaacagcattcagtaatagtgatgcaataaaagcacttcagcttgcctcctctggcatgcaggccgctcaggtttttggcgcggccataggaggatgggctatcagtacgggttactacatgcaagcgctcatgctgacctgctgcatctaccttttgaatatttatatttcgacactttttgagaaagggaatcctgataataaagacgctgaggttattgtgcagattcactctgtcactaagaaaaatgaatcctttacatatctttttcgcagtaaggactttttgttacccttgatttttactgttccatcaagtggagctttacaatttttaaacacatcccttccttcattatcttctctatatggtaatagcgagaaaatttatccagtgctgaatatgactctacaatgtgctgtcattatctcggggatagcagcagcactcaatatgctttctctaaagtcttcattacggttttctctgtgtatttctggcatctgtctgattttgatgtgtttatcatcgagaaattattatgccgtatacttttttttatttctcacttctttttttgtttcatggcatatgatttctattaaggtactcacgaatcaaatgcctgatatagaaaatattggtaaattcacaatgatgagaaactcagtcgcttctggtgtgaaaattgtcttttctatttcgtctggtgcttttttgactttttacagcataactacaacttatctaatacttgcgatactgcttatttttttcaatgtactatgggtttgtcagagtcgcagttttaactatgaggatttaggtgatgttaaatttcaaaagtga

SEQ ID NO: 10 - DNA, 1008 bp, Pantoea ananatis
  source 1..1008: mol_type = other DNA
atgttaaatttcaaaagtgatttttctgaatttattactggtttttatcttaaacagtttactcatttaaatacgcaggaaagagaacatgtgcttgaaacgcttggcgtcacacccagcgcaatcaatgagtttataacatcagaagatatttatattaccttacctcatgcaagtatgaatgttttttttccaagagcgggggtctcacgttatgtctacgatctacaaaatagtaacaaaaacgcgttccatttaaggtttttcttgacccataccaactttagcgatctgaactggcgcccttatgcatggtggtttaataacggcgggaaaatagataaacttacctttttcacacgaaataaaaaaaagaaacataatatcgtttactcgctaaaaccaaatgaaatgcactctgcctctgtagatcgacgtctgcgtcatgactttgatacaagtatgaagtttaaacgtatttctcttagcttcatctatatgacagccgttcaagaggtcaatagcggttttgcacataaaggccgtaccctttatctcccacttgacgcatttgtagcttttattattcatcaggcaaaaaacgatgttatatcttttaattttttagaagcttttttaagccaggcacaatgcagaagactgaatggcgaagaattgagttttaccagtgaatggcgcgatgcatttatctttgataattttacaaacattgcgctgttaaatttttttcagcctgcagcatttgtgggaggcgaaaaaatggataactactggcatcaggtgattgaaaaatggaaaatggcattaccaaatcaatctgaagttgagtttgcgttacccactaatctggtcatgcctgccgttcaagagtatatttacccttataaaccatcaagcgatatagcagaacagcttattaaaaataacatcccatacagtcttaccatggctattcaggaacatgatcttttttcaaagaaagaataa

SEQ ID NO: 11 - DNA, 480 bp, Pantoea ananatis
  source 1..480: mol_type = other DNA
atgctagataaaagtgcttttcgccacgcaatgtcgcatcttcctactgcagtcactattgtaacatcaagcggctcctgcggggctgcagcatgtacagtttcatccgtgtgttctgtcaccgacgacccacccactttactcgtatgtataaatcgtgcctccaataacaatagtgttataagaaataatggctctttgtgtgtcagcattctttcaggagagcaaagcaacattgcaatgcaatgtgctaatcatcaggtttcagttgaggagcgcttagcattattcgacgcagacgttctggtcacggggtctcctgctgtagttgattccgtttgctgtttagattgtcgtgtagataatattgtggaatcaggaacccactctgtcttcttttgtcaggttctttcttctaaagcttttgtggggaaagatgcgctggtttattattgccggaattatcatcgtgtatcataa

SEQ ID NO: 12 - DNA, 1155 bp, Pantoea ananatis
  source 1..1155: mol_type = other DNA
atgatgaaaagtttaattattacgtcgcattttaatagtgatttatcctctatggcatgcgatgagtttggattaaaaagatcactaattttctatactccaaaaataaatgaacttagcttaggtttaaaatatgagtactacgataagatagattcttactctgtaagattaatggagcttgggaatgaaattgattttatatggagcacttccggttctgaaatcaactcgttcgctgaattaaaagctgttgagttggcaggattaagaaacatctatctaggcatgaatgaaaatgctttttctacagctgcttataaaagcatcacgcaagagttaatggcatttttgggtgttagtattcccaaagggcttcaatgtaacacaaaaaaagaaatacatgatttccttgaatataatggtggaactatagtctgtaaagctaacaatggagcaggtggtgtaaatcaattttattgccaaagatttgatgacatatataaactccctcatgaggtaactgattggtatgtagaacagtttctgaaaggacttgagttttctgtaaacgcttatatgctaaatgggttttatatagcctccccgataatgtttaaaggcgaaactgatattcatagcgggcacgcaatggacaaattcaggtatattagcaagctaaaaaataaaagcttaaatgaaaaaataaatgccatattaagtaaaattagtaataccaatatctttaatggatggatagaggttgagtttataaaaacacatcaggatttggtagttatagaaattaatgcgaggtataatggaacaatcagagctactggatatgcttgtaatgaaaatttatatcaattggatttggaaagtaaaatttataataaattttcttcacaattaaatcatgaaaacgaagtcattgagatgccaattcatttgaaacttgaaacaggtttaaaagaatttggtttcgtccaaaaaatgaaatctcgaaaaaccaacacaggaagggccacaatatggggtgaagatcaatgtgaactattagagaggatcaaaaatactgaattagaaatatactcagaaagaattattcatggcataaatgagagtaaggagttattcgaaaaatatatttaa

SEQ ID NO: 13 - DNA, 658 bp, synthetic construct
  misc_feature 1..658: note = Description of Artificial Sequence: Synthetic polynucleotide
  source 1..658: mol_type = other DNA
gcgcaacgcaattaatgtgagttagctcactcattaggcaccccaggctttacactttatgcttccgggttttctttgcctcacgatcgcccccaaaacacataaccaattgtatttattgaaaaataaatagatacaactcactaaacatagcaattcagatccggtccagtaatgacctcagaactccatctggatttgttcagaacgctcggttgccgccgggcgttttttattggtgagaatcgcagcaacttgtcgcgccaatcgagccatgtcgtcgtcaacgaccccccattcaagaacagcaagcagcattgagaactttggaatccagtccctcttccacctgctgatccggatcgatcccgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattgatccggccaagcttgtaagaaggagatatacat

SEQ ID NO: 14 - protein, 284 aa, Pantoea ananatis
MIKKLIAEKGTLIFIEAHNPLSALIASKAEQTNSEGRIVKFDGIWSSSLTDSASRGIPDNETLALSSRLENIADIRNVTDMPIIMDADTGGKPEHFSYYVKRMINNGVNGVIIEDKTGLKKNSLFGTEVEQTLADINDFSEKIKRGKSAVYIDDFMIIARLESLIAGFDVEHALERADAYVEAGADGIMIHSCKKTPDEVFLFSTKFRKKYPSVPLICVPTTYSATSNRELSEAGFNVIIYANHMLRAAYKAMENVSKEILRYGRTAEIEKSCMSVKEIISLIP

SEQ ID NO: 15 - protein, 459 aa, Pantoea ananatis
MHECKGNYFTDSLKRMTMKKKEMIIGAYISYGTGHHPASWRESGVNAAAALDIDTYANLARVCEKGLADLLFLADTPSVFQDNMDGYGSRVSVLEPLSLLSYLASQTQNIGLVATASTTYKHPYNIAREFASLDYISKGRAGWNLVTSSKSDAAKNFGLAAHPEHSKRYDMAWEAWQVISGLWDSWEDNALVRNKTSGQFFVKDKYREINFEGEYFNVKGPLNIARPPQGYPVIVQAGSSEEGKELAAKTADIVFTAQNNIEDAKKFYDDLKGRMEKYGRSKSELLILPGLSFYIASDESKARKKLNDLNALIPQSFGLSMLSDLLGGVDLKNNDPEGPLPDLPKSNGNQSRQKIIIDLARKEKLSIKQLYEKIIISRGHYTFTGSYQDLADEMIKWVENEACDGFNIMPPLMPESLINLFDHVIPLIQARGWYKKSYSTGTLREKLGLKRPTNKLFNQ

SEQ ID NO: 16 - protein, 373 aa, Pantoea ananatis
MLNKNLILEDTTLRDGEQAPGVAFTPEQKVEIFYLLANMGVKWIEAGIPAMKGDEVKALSEMLERKNEINIIAWNRGVLEDIEYSISLGFKAVHIGLPTSAIHLEKSVKKDKSWLVKTASDLVKFAKDKGMFVSISAEDIGRTDIGFLQEYAQVVAEAGADRLRLSDTIGILSPAQYKEKVSLLNKNVNIDLQCHCHNDFGFAVANTLAGIEAGARYFHVCVNGIGERAGMPDLAQVAMALHFFHGVDLGLDLTKLIALSETVARYSHQKISPWQPIVGDNVFAHESGIHANGMLKDSSTFEPFDPATVGGERRLVVGKHSGRAIIKHFLEESGVKAADDKALDRCLERVRSHAVRHPGGIPPHVLVDLYTAG

SEQ ID NO: 17 - protein, 412 aa, Pantoea ananatis
MKSNQPIVNQIIASHSGRGQVSAGELITVDVDYVYVQDGNSPTVAKLFQDYHLSEVLKPDKIGFFFDHSVLVPDKTMAKRVNEAMEFAKKLGINIYSRGEGISHVIALESKIFKPGNIVLGADSHTCTGGAVQSLALGMGASDILVAMLTGQTWLKVPQTVHLCIKGKTGKDVRAKDVMLALLNKYGQTPFLYKSIEVSGEWAEELTLDEAASFASMAVELGAKCIFMPDGQGRPEGLLKADASVADSVINFSVSELMPHIAPPHSPLYAKPANDFEGLKFDYIFIGSCTNSRLEDIKEVAEIVAGKTIHPDIHCLLTPGSKSVYLKALQAGYIDTLIRSGIIVTPPGCGACVGTQGTIPADGEKVLSTMNRNFKGRMGNAEADIFLCSPRTAAMVALNGTVPHFEGESAYE

SEQ ID NO: 18 - protein, 187 aa, Pantoea ananatis
MFHILRESPHMSKTAENYRVRRVEGNISTDDIIPARYKHMYTEPAQLAPHLFESRFPGFRETLSINDVLVCDQIFGIGSSREQAVTTLLACGVKYVFSPSFGRIFFRNSWNLGLHAIEVDTSELADLSEIKIELTGGVIYTENNQINFFPPSSQMTAIVSAGGIIPYTINKIMEKKGDILRGYSNEK

SEQ ID NO: 19 - protein, 255 aa, Pantoea ananatis
MKSEKFDGLADNYDKYRPRYPAILFKEIHDWMQPSAKNIYDIGAGTGIAIEGMTRVTGKHYDFTAIDISEDMIKKGREKLPGTTWVKGKAEDILSDKSRIDVIMAAQSFQWMDRAKTLEVSIKSLNKGGVFAVLQNNRDYRNNEMLNKYEGLLEKFSPGYSRHYRDYDYENEITNVFKLPIANFKKVVTGWTMEMISEDFFGFISSSTQVQRAIENDRNGFWKEIEILIDEHSVGGKISIDYISELFIAKKRDDS

SEQ ID NO: 20 - protein, 158 aa, Pantoea ananatis
MIHSMTIQDISIEQASYEDAKLLRKALEKVYEPYTLNFSPTALQFTENIIAQESSKWLVAKYKSDIVGAVRYELYDIYLDFHFLCVTPPFRKMGVGNELFHKLKKIAYEKRKDFMKIVLRDSLSYNRRYFESKGFYFYHKYQTNMHSVFILKLNGEKP

SEQ ID NO: 21 - protein, 367 aa, Pantoea ananatis
MNKKALVIGLKSNMERVIKGLNEIEFIIIDRGTLDNESIDYIINLSDDLMHKKSFSYVIASSEDFIALAGLLRNRYSLYGEKYYKSTIATNKFLMRNFCSGFLSCPKFWLSGEIINSENLLLSSQKDYIVKPLTGSSAKHVETVTQEDLNQYLNENNKLMLIEEKVLMRDEYHLDCIIKDGNILFSTLSIYDRPILEAKSKNRASINLPDGTRLHEDALILATNLQSHFEMTNGVFHIEMYHTQDGFILGEFGIRPPGAGVTDMYYMYRGVDFWEAFIYSQIDKEFILPLNHKSDKYCAAIGICSSFPVDDIRSTSKVSVDKYVNLRENAAKPAVPSSTSFNHMIYVSSSSLEEIRNFLHDISDRQE

SEQ ID NO: 22 - protein, 412 aa, Pantoea ananatis
MLKYETKKNAFFQIYNFLSCSAEGIQTVIFLWLIYHETHSPMLVSLTIVSSYLPSAVLGFFFLKKADASSPGKQLFISNVSLSAISLIVYFILMRNEGFELITLSIFYLAQAVLSVVKMFNKTSQNRIIRTAFSNSDAIKALQLASSGMQAAQVFGAAIGGWAISTGYYMQALMLTCCIYLLNIYISTLFEKGNPDNKDAEVIVQIHSVTKKNESFTYLFRSKDFLLPLIFTVPSSGALQFLNTSLPSLSSLYGNSEKIYPVLNMTLQCAVIISGIAAALNMLSLKSSLRFSLCISGICLILMCLSSRNYYAVYFFLFLTSFFVSWHMISIKVLTNQMPDIENIGKFTMMRNSVASGVKIVFSISSGAFLTFYSITTTYLILAILLIFFNVLWVCQSRSFNYEDLGDVKFQK

SEQ ID NO: 23 - protein, 335 aa, Pantoea ananatis
MLNFKSDFSEFITGFYLKQFTHLNTQEREHVLETLGVTPSAINEFITSEDIYITLPHASMNVFFPRAGVSRYVYDLQNSNKNAFHLRFFLTHTNFSDLNWRPYAWWFNNGGKIDKLTFFTRNKKKKHNIVYSLKPNEMHSASVDRRLRHDFDTSMKFKRISLSFIYMTAVQEVNSGFAHKGRTLYLPLDAFVAFIIHQAKNDVISFNFLEAFLSQAQCRRLNGEELSFTSEWRDAFIFDNFTNIALLNFFQPAAFVGGEKMDNYWHQVIEKWKMALPNQSEVEFALPTNLVMPAVQEYIYPYKPSSDIAEQLIKNNIPYSLTMAIQEHDLFSKKE

SEQ ID NO: 24 - protein, 159 aa, Pantoea ananatis
MLDKSAFRHAMSHLPTAVTIVTSSGSCGAAACTVSSVCSVTDDPPTLLVCINRASNNNSVIRNNGSLCVSILSGEQSNIAMQCANHQVSVEERLALFDADVLVTGSPAVVDSVCCLDCRVDNIVESGTHSVFFCQVLSSKAFVGKDALVYYCRNYHRVS

SEQ ID NO: 25 - protein, 384 aa, Pantoea ananatis
MMKSLIITSHFNSDLSSMACDEFGLKRSLIFYTPKINELSLGLKYEYYDKIDSYSVRLMELGNEIDFIWSTSGSEINSFAELKAVELAGLRNIYLGMNENAFSTAAYKSITQELMAFLGVSIPKGLQCNTKKEIHDFLEYNGGTIVCKANNGAGGVNQFYCQRFDDIYKLPHEVTDWYVEQFLKGLEFSVNAYMLNGFYIASPIMFKGETDIHSGHAMDKFRYISKLKNKSLNEKINAILSKISNTNIFNGWIEVEFIKTHQDLVVIEINARYNGTIRATGYACNENLYQLDLESKIYNKFSSQLNHENEVIEMPIHLKLETGLKEFGFVQKMKSRKTNTGRATIWGEDQCELLERIKNTELEIYSERIIHGINESKELFEKYI

SEQ ID NO: 26 - DNA, 18 bp, synthetic construct
  misc_feature 1..18: note = Description of Artificial Sequence: Synthetic primer
  source 1..18: mol_type = other DNA
cttgctgcaggtaggggt

SEQ ID NO: 27 - DNA, 20 bp, synthetic construct
  misc_feature 1..20: note = Description of Artificial Sequence: Synthetic primer
  source 1..20: mol_type = other DNA
tctatccacggcaaaccact

SEQ ID NO: 28 - DNA, 18 bp, synthetic construct
  misc_feature 1..18: note = Description of Artificial Sequence: Synthetic primer
  source 1..18: mol_type = other DNA
tgatggcctgcaagacgg

SEQ ID NO: 29 - DNA, 20 bp, synthetic construct
  misc_feature 1..20: note = Description of Artificial Sequence: Synthetic primer
  source 1..20: mol_type = other DNA
tctatccacggcaaaccact

SEQ ID NO: 30 - DNA, 18 bp, synthetic construct
  misc_feature 1..18: note = Description of Artificial Sequence: Synthetic primer
  source 1..18: mol_type = other DNA
cagcgcaacagactggga

SEQ ID NO: 31 - DNA, 18 bp, synthetic construct
  misc_feature 1..18: note = Description of Artificial Sequence: Synthetic primer
  source 1..18: mol_type = other DNA
cccattccgccatgagca

SEQ ID NO: 32 - DNA, 18 bp, synthetic construct
  misc_feature 1..18: note = Description of Artificial Sequence: Synthetic primer
  source 1..18: mol_type = other DNA
acggtaagattgggcgcc

SEQ ID NO: 33 - DNA, 18 bp, synthetic construct
  misc_feature 1..18: note = Description of Artificial Sequence: Synthetic primer
  source 1..18: mol_type = other DNA
ggccaacgatcgcggata

SEQ ID NO: 34 - DNA, 22 bp, synthetic construct
  misc_feature 1..22: note = Description of Artificial Sequence: Synthetic primer
  source 1..22: mol_type = other DNA
gctgctatccccgagataatga

SEQ ID NO: 35 - DNA, 22 bp, synthetic construct
  misc_feature 1..22: note = Description of Artificial Sequence: Synthetic primer
  source 1..22: mol_type = other DNA
gctgaagggattcagacggtta

SEQ ID NO: 36 - DNA, 18 bp, synthetic construct
  misc_feature 1..18: note = Description of Artificial Sequence: Synthetic primer
  source 1..18: mol_type = other DNA
ttaccgccaccttgctgg

SEQ ID NO: 37 - DNA, 18 bp, synthetic construct
  misc_feature 1..18: note = Description of Artificial Sequence: Synthetic primer
  source 1..18: mol_type = other DNA
tttcgcccgttccccttc

SEQ ID NO: 38 - DNA, 20 bp, synthetic construct
  misc_feature 1..20: note = Description of Artificial Sequence: Synthetic primer
  source 1..20: mol_type = other DNA
gccgtcctgccatatctcaa

SEQ ID NO: 39 - DNA, 20 bp, synthetic construct
  misc_feature 1..20: note = Description of Artificial Sequence: Synthetic primer
  source 1..20: mol_type = other DNA
taacggactcagcatctcgc

SEQ ID NO: 40 - DNA, 18 bp, synthetic construct
  misc_feature 1..18: note = Description of Artificial Sequence: Synthetic primer
  source 1..18: mol_type = other DNA
cccgacccgaaacaccat

SEQ ID NO: 41 - DNA, 18 bp, synthetic construct
  misc_feature 1..18: note = Description of Artificial Sequence: Synthetic primer
  source 1..18: mol_type = other DNA
tgttcgccaggctcaagg

SEQ ID NO: 42 - DNA, 44 bp, synthetic construct
  misc_feature 1..44: note = Description of Artificial Sequence: Synthetic primer
  source 1..44: mol_type = other DNA
actaaagggaacaaaagctggagctccgccaaaaatcagctgtg

SEQ ID NO: 43 - DNA, 31 bp, synthetic construct
  misc_feature 1..31: note = Description of Artificial Sequence: Synthetic primer
  source 1..31: mol_type = other DNA
ggatttaattgtgaaagactctccgctcgtg

SEQ ID NO: 44 - DNA, 41 bp, synthetic construct
  misc_feature 1..41: note = Description of Artificial Sequence: Synthetic primer
  source 1..41: mol_type = other DNA
gcggagagtctttcacaattaaatcctcacatcagtagagg

SEQ ID NO: 45 - DNA, 51 bp, synthetic construct
  misc_feature 1..51: note = Description of Artificial Sequence: Synthetic primer
  source 1..51: mol_type = other DNA
ctggatgatcctccagcggggccccccctcgagttcatggcgcggctttcg

SEQ ID NO: 46 - DNA, 36 bp, synthetic construct
  misc_feature 1..36: note = Description of Artificial Sequence: Synthetic primer
  source 1..36: mol_type = other DNA
attttatttatttaaacgttaaacaagaaattcatc

SEQ ID NO: 47 - DNA, 43 bp, synthetic construct
  misc_feature 1..43: note = Description of Artificial Sequence: Synthetic primer
  source 1..43: mol_type = other DNA
tcctccagcggggccccccctcgagttgaagcggctaacttcc

SEQ ID NO: 48 - DNA, 40 bp, synthetic construct
  misc_feature 1..40: note = Description of Artificial Sequence: Synthetic primer
  source 1..40: mol_type = other DNA
agggaacaaaagctggagctcttgtttcatccatcatacc

SEQ ID NO: 49 - DNA, 35 bp, synthetic construct
  misc_feature 1..35: note = Description of Artificial Sequence: Synthetic primer
  source 1..35: mol_type = other DNA
ttaacgtttaaataaataaaattgcttgtctcatg

SEQ ID NO: 50 - DNA, 33 bp, synthetic construct
  misc_feature 1..33: note = Description of Artificial Sequence: Synthetic primer
  source 1..33: mol_type = other DNA
ctgacggattttacaaacgcaaaaacccccgcc

SEQ ID NO: 51 - DNA, 51 bp, synthetic construct
  misc_feature 1..51: note = Description of Artificial Sequence: Synthetic primer
  source 1..51: mol_type = other DNA
ctggatgatcctccagcggggccccccctcgagtaatcgccgcccacgccg

SEQ ID NO: 52 - DNA, 45 bp, synthetic construct
  misc_feature 1..45: note = Description of Artificial Sequence: Synthetic primer
  source 1..45: mol_type = other DNA
actaaagggaacaaaagctggagctcccatcattacgtttatgcc

SEQ ID NO: 53 - DNA, 32 bp, synthetic construct
  misc_feature 1..32: note = Description of Artificial Sequence: Synthetic primer
  source 1..32: mol_type = other DNA
gtttttgcgtttgtaaaatccgtcaggtgcac

SEQ ID NO: 54 - DNA, 18 bp, synthetic construct
  misc_feature 1..18: note = Description of Artificial Sequence: Synthetic primer
  source 1..18: mol_type = other DNA
actatgagcacgtcggcg

SEQ ID NO: 55 - DNA, 18 bp, synthetic construct
  misc_feature 1..18: note = Description of Artificial Sequence: Synthetic primer
  source 1..18: mol_type = other DNA
cgcactcccgttctggat

SEQ ID NO: 56 - DNA, 20 bp, synthetic construct
  misc_feature 1..20: note = Description of Artificial Sequence: Synthetic primer
  source 1..20: mol_type = other DNA
caggatgaggatcgtttcgc

SEQ ID NO: 57 - DNA, 18 bp, synthetic construct
  misc_feature 1..18: note = Description of Artificial Sequence: Synthetic primer
  source 1..18: mol_type = other DNA
tcgaaccccagagtcccg

SEQ ID NO: 58 - DNA, 28 bp, synthetic construct
  misc_feature 1..28: note = Description of Artificial Sequence: Synthetic primer
  source 1..28: mol_type = other DNA
atgtatatctccttcttacaagcttggc

SEQ ID NO: 59 - DNA, 31 bp, synthetic construct
  misc_feature 1..31: note = Description of Artificial Sequence: Synthetic primer
  source 1..31: mol_type = other DNA
tctagatgcactccaccgctgatgacatcag

SEQ ID NO: 60 - DNA, 52 bp, synthetic construct
  misc_feature 1..52: note = Description of Artificial Sequence: Synthetic primer
  source 1..52: mol_type = other DNA
atgtcatcagcggtggagtgcatctagattaaggaatcagtgaaataatttc

SEQ ID NO: 61 - DNA, 47 bp, synthetic construct
  misc_feature 1..47: note = Description of Artificial Sequence: Synthetic primer
  source 1..47: mol_type = other DNA
aagcttgtaagaaggagatatacatatgatcaaaaaacttattgcag

SEQ ID NO: 62 - DNA, 49 bp, synthetic construct
  misc_feature 1..49: note = Description of Artificial Sequence: Synthetic primer
  source 1..49: mol_type = other DNA
catgagaattaattccggggatccgtcgacactaccatcgggggccatc

SEQ ID NO: 63 - DNA, 49 bp, synthetic construct
  misc_feature 1..49: note = Description of Artificial Sequence: Synthetic primer
  source 1..49: mol_type = other DNA
ctacagcctcgggaattgctgcaggtcgactctagatgcactccaccgc

(19) United States
(12) Patent Application Publication
(10) Pub. No.: US 2025/0282802 A1
(43) Pub. Date: Sep. 11, 2025

(54) PHOSPHONATE PRODUCTS AND METHODS

(71) Applicant: THE BOARD OF TRUSTEES OF THE UNIVERSITY OF ILLINOIS, Urbana, IL (US)

(72) Inventors: William W. METCALF, Savoy, IL (US); Alexander L. POLIDORE, Champaign, IL (US)

(73) Assignee: THE BOARD OF TRUSTEES OF THE UNIVERSITY OF ILLINOIS, Urbana, IL (US)

(21) Appl. No.: 19/093,316

(22) Filed: Mar. 28, 2025

Related U.S. Application Data
(63) Continuation of application No. 18/043,987, filed on Mar. 3, 2023, now Pat. No. 12,264,172.
(60) Provisional application No. 63/075,138, filed on Sep. 5, 2020.
(60) Provisional application No. 63/181,745, filed on Apr. 29, 2021.

(51) Int. Cl. C07F 9/38; A01N 57/20

(52) U.S. Cl. CPC C07F 9/3826; A01N 57/20

(57) ABSTRACT

P. ananatis produces at least three phosphonates, two of which were purified and structurally characterized. The first, designated pantaphos, was shown to be 2-(hydroxy(phosphono)methyl)maleate; the second, a probable biosynthetic precursor, was shown to be 2-(phosphonomethyl)maleate. Purified pantaphos is both necessary and sufficient for the hallmark lesions of onion center rot. Moreover, when tested against mustard seedlings, the phytotoxic activity of pantaphos was comparable to the widely used herbicides glyphosate and phosphinothricin. Pantaphos was also active against a variety of human cell lines.

### RELATED APPLICATIONS

This application is a continuation of U.S. application Ser. No. 18/043,987 filed Mar. 3, 2023, which is a National Stage filing under 35 U.S.C. § 371 of International Application No. PCT/US2021/048904 filed Sep. 2, 2021, which claims the benefit of U.S. Provisional Patent Application No. 63/181,745, filed Apr. 29, 2021, and U.S. Provisional Patent Application No. 63/075,138, filed Sep. 5, 2020, which applications are incorporated herein by reference.

### GOVERNMENT SUPPORT

This invention was made with government support under Grant No. R01 GM127659 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Feb. 6, 2025, is named 500.127US2_SL and is 88,137 bytes in size.

### BACKGROUND OF THE INVENTION

Pantoea species have been recognized as plant pathogens since 1928. These gram-negative Enterobacteriaceae were originally classified as members of the genus Erwinia, but were subsequently moved to Pantoea based on DNA hybridization experiments. Although many Pantoea species are benign or beneficial plant mutualists, strains of P. ananatis are consistently associated with harmful crop and forest infestations. Since 1983, the known hosts of P. ananatis have increased to eight plant species in 11 countries, including important crops such as rice, corn, onions, melon, and pineapple. Upon plant infection, these bacteria cause internal rotting, dieback, and blight resulting in severe economic losses. In addition to primary infection in the field, significant post-harvest losses, as observed in onion center rot, have also been reported. Moreover, this plant pathogen can also infect humans and insects, which serve as vectors for plant infection. Thus, there is a compelling need to understand P. ananatis pathogenesis to help address their epidemic spread among essential food crops.

Despite the economic and food safety implications of P. ananatis infection, the mechanisms of plant pathogenicity have only recently been investigated. Comparative genomic analyses revealed substantial diversity between P. ananatis strains, which may account for their ability to colonize and thrive in so many different hosts. The pathogenicity determinants encoded by diverse P. ananatis genomes include quorum sensing systems, type VI secretion systems, motility factors, cell-wall degrading enzymes and thiosulfinate resistance alleles. A novel pathogenicity determinant for onion center rot was recently revealed by comparison of the genomic sequences of two pathogenic and two non-pathogenic P. ananatis strains (Mol Plant Microbe Interact 2018, 31:1291).

This approach identified a genomic island designated “HiVir”, which was subsequently shown to be present in fourteen pathogenic strains and absent in sixteen non-pathogenic strains using a PCR-based screen. The HiVir locus encodes an eleven-gene operon (hereafter designated hvr) that was suggested to encode a biosynthetic pathway for an unknown phosphonic acid natural product based on the presence of a putative pepM gene. This gene encodes the enzyme phosphoenolpyruvate (PEP) phosphonomutase, which catalyzes the first step in all characterized phosphonate biosynthetic pathways, and which has been extensively used as a genetic marker for the ability to produce phosphonic acid metabolites. Deletion of pepM in P. ananatis OC5a resulted in a strain with severely attenuated pathogenicity in Allium cepa (onion), demonstrating a required role for the hvr operon in onion center rot. Based on this finding, Asselin et al suggested that a small molecule phosphonate is involved in plant disease caused by P. ananatis.

Phosphonates, defined by the presence of chemically stable carbon-phosphorus bonds, are an underdeveloped class of bioactive molecules with significant applications in both medicine and agriculture. The bioactivity of these molecules results from their structural similarity to phosphate esters and carboxylic acids, which allows them to bind enzymes that act on analogous substrates, thus inhibiting enzyme activity. A prominent example is the manmade herbicide glyphosate, which was first synthesized by chemists in the 1950's. The phytotoxicity of glyphosate is due to its inhibition of 5-enolpyruvylshikimate-3-phosphate (EPSP) synthase, a key enzyme in the biosynthesis of aromatic amino acids in plants. Significantly, enzyme inhibition by individual phosphonates is quite specific and typically confined to enzymes that act on chemically homologous substrates. Accordingly, a phosphonate can be toxic to one group of organisms, while remaining innocuous to another. Thus, depending on the organism in which the target enzyme is found, phosphonates find applications as specific antibacterial, antifungal, antiparasital and herbicidal compounds. Given the ubiquitous occurrence of phosphate esters and carboxylic acids in metabolism, the range of potential biological targets for phosphonate inhibitors is vast. Indeed, as demonstrated by the number of organisms known to produce bioactive phosphonates, nature has often capitalized on this metabolic Achilles heel. Examples include phosphinothricin tripeptide and fosmidomycin, produced by members of the genus Streptomyces, which have potent herbicidal and antimicrobial activity due to their inhibition of the essential enzyme's glutamate synthase and deoxyxylulose-5-phosphate reductoisomerase, respectively. Nature also makes use of the fact that the C—P bond is highly stable and resistant to both chemical and enzymatic degradation. Accordingly, many organisms replace labile biomolecules such as phospholipids and phosphate ester-modified exopolysaccharides with analogous phosphonates.

Considering their useful biological properties, it is not surprising that biosynthesis of phosphonate compounds is common among microbes. Based on the presence of pepMin sequenced genomes and metagenomes, ca. 5% of all bacteria possess the capacity for phosphonate biosynthesis. Biosynthetic gene clusters that include pepM are known to direct the biosynthesis of phosphonolipids, phosphonoglycans, and a wide variety of small molecule secondary metabolites. Like the streptomycete-derived natural products described above, many of these small molecule phosphonates are bioactive. Although considerable progress has been made in understanding the bioactivity and biosynthesis of small molecule phosphonates, only a fraction of the observed pepM-encoding gene clusters have been characterized. Thus, the extent of phosphonate chemical diversity in nature has yet to be established.

Consistent with the idea that phosphonate biosynthesis is common in nature, it has also been observed that about 30% of sequenced bacterial genomes contain genes for phosphonate catabolism, which allows their use as sources of phosphorus, carbon or nitrogen. Genes encoding the carbon-phosphorus (C—P) lyase system, which catalyzes a multi-step phosphonate degradation pathway with broad substrate specificity, are particularly common in bacteria. Other examples include the enzyme phosphonatase, which is specific for aminoethylphosphonate, and a recently characterized oxidative pathway for use of hydroxymethylphosphonate.

One of the challenges for agriculture is the emerging resistance to synthetic herbicides and the lack of novel, effective natural product herbicides. It is estimated that only 7% of conventional pest control agents (includes insecticides, fungicides, and herbicides) are natural products or natural products derived. However, in the case of herbicidal compounds, only one class of natural product-derived herbicide has been registered since 1997, and compared to 30% of fungicides and insecticides, only 8% of herbicidal compounds are natural product-derived. Derivatives of this natural product produced from this strain can be an alternative organic treatment strategy for herbicide-resistant crops.

The problem is the increased resistance to commercially available herbicides poses a threat to the agriculture industry. Accordingly, there is in need for the development of novel herbicides to combat this resistance.

### SUMMARY

Pantoea ananatis is a significant plant pathogen that targets a number of important crops, a problem that is compounded by the absence of effective treatments to prevent its spread. Our identification of pantaphos as the key virulence factor in onion center rot suggests a variety of approaches that could be employed to address this significant plant disease. Moreover, the general phytotoxicty of the molecule suggests that it could be developed into an effective herbicide to counter the alarming rise in herbicide-resistant weeds.

Accordingly, this disclosure provides a composition comprising a compound of Formula I:

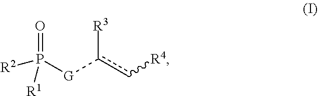

or a salt thereof, wherein
   [symbol] represents single or double bond;
   [symbol] represents double or single bond, wherein both [symbol] and [symbol] are not double bonds;
   G is X^{A}CHOR⁵, O, C(═O), C(═CH₂), CHP(═O)(R⁶)₂, or CX^{B}₂;
   X^{A} is absent or O;
   each X^{B} is independently H or halo;
   R¹ and R² are each independently OR^{A} or an amino acid;
   R³ is —C(═O)R⁷ or a triazole or tetrazole;
   R⁴ is —C(═O)R⁸ or a triazole or tetrazole;
   R⁵ is H, —(C₁-C₆)alkyl, —(C₃-C₆)cycloalkyl, aryl, or heteroaryl;
   each R⁶ is independently OR^{B} or an amino acid;
   R⁷ and R⁸ are each independently OR^{C} or an amino acid; and
   each R^{A}, R^{B} and R^{C} are independently H, —(C₁-C₆)alkyl, —(C₃-C₆)cycloalkyl, aryl, or heteroaryl; and
a non-aqueous fluid, additive or combination thereof.

This disclosure also provides a method for inhibiting growth or formation of a weed comprising contacting the weed and/or soil where the weed can form and a herbicidally effective amount of a composition or compound disclosed herein, wherein growth or formation of the weed is inhibited.

Also, this disclosure provides a method for inhibiting growth of a cancer cell, treatment of cancer in a subject in need of cancer therapy. Additionally, this disclosure provides a method for forming or manufacturing 2-(hydroxy(phosphono)methyl)maleic acid and 2-phosphono-methylmaleate.

The technology described herein provides novel compounds of Formula I and Formula II, intermediates for the synthesis of compounds of Formula I and Formula II, as well as methods of preparing compounds of Formula I and II. The technology also provides compounds of Formula I and II that are useful as intermediates for the synthesis of other useful compounds. The technology provides for the use of compounds of Formula I and Formula II for the manufacture of medicaments useful for the treatment of cancer in a mammal, such as a human.

The technology provides for the use of the compositions described herein for use in medical therapy or as herbicides. The medical therapy can be treating cancer, for example, brain cancer, breast cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Onion center rot phenotype of P. ananatis strains used in the study. Surface sterilized onion bulbs were inoculated as indicated in each panel, then incubated at 30° C. for fourteen days prior to sectioning to reveal the center rot phenotype. Panels A-C show inoculations of a sterile water control and wild-type strains. Panels D-J show inoculations with mutant derivatives of P. ananatis LMG 5342.

FIG. 2. Construction of a P. ananatis LMG 5342 derivative with IPTG-inducible hvr expression. Plasmid pAP01, which carries the hvrA gene under control of the IPTG-inducible Ptac promoter, was transferred to P. ananatis MMG1998 via conjugation from an E. coli donor. Because pAP01 is incapable of autonomous replication in P. ananatis, kanamycin-resistant (conferred by the aph gene) excongugants can only be obtained by chromosomal integration of the plasmid via homologous recombination (depicted as dotted lines). The resulting strain, P. ananatis MMG2010, expresses the entire hvr operon from the Ptac promoter. The positions of the native hvr promoters are shown as unlabeled bent arrows.

FIG. 3. Production of phosphonates is correlated with expression of the hvr operon. P. ananatis MMG2010, which expresses the hvr operon from an IPTG-inducible promoter, was grown in glycerol minimal medium with and without IPTG. Spent media were then concentrated and analyzed by ³¹P NMR. Phosphonic acids typically produce peaks in the 5-30 ppm range, whereas peaks from phosphate and its esters and anhydrides typically have chemical shifts in the <5 ppm range.

FIG. 4. Chemical complementation of the Ahvr onion rot phenotype by P. ananatis phosphonates. Surface sterilized onion bulbs were inoculated as indicated in each panel, then incubated at 300° C. for fourteen days prior to sectioning to reveal the center rot phenotype. Panels A and B show inoculations of a sterile water control and the Ahvr mutant. Panels C and D show inoculations with the hvr mutant supplemented with spent medium from an IPTG-induced culture of the phosphonate producing strain P. ananatis MMG2010 or purified pantaphos. Panels E-F show inoculations of onion with sterile spent medium or purified pantaphos in the absence of bacteria.

FIG. 5. Phytotoxicity of pantaphos compared to known herbicides. Mustard seedlings were treated as indicated and incubated at 23° C. under 16-hour light cycle for seven days. Panel A shows the observed phenotype after incubation with the indicated compounds. Panels B and C show root length and dry weight of each replicate after incubation. A Welch's t-test was performed to establish significant differences between the means of each treatment (***P-value <0.001, **P-value <0.01, *P-value <0.05; N=6 per treatment). Error bars represent the standard error of the mean.

FIG. 6. Phytotoxicity of pantaphos against Arabidopsis thaliana Col-0. A. thaliana Col-0 seedlings were treated as indicated and incubated at 23° C. at 60% humidity under 16-hour light cycle for seven days. A Welch's t-test was performed to calculate statistics between the means of the different treatments (***P-value <0.001, **P-value <0.01, *P-value <0.05; N=6 per treatment). Error bars represent the standard error of the mean.

FIG. 7. Hvr biosynthetic gene cluster in P. ananatis LMG 5342 and the proposed biosynthetic pathway. The proposed protein functions of the Hvr BGC genes based on BLAST searches and conserved protein domain analyses.

FIG. 8. Cytotoxicity of pantaphos across various cancer cell lines. Assays were conducted as described (mBio. 2021 Feb. 2; 12(1):e03402-20), using the Alamar Blue method with 72-hour treatment using Raptinal (50 mM) as Dead cell control; n=3. Cell seeding densities: CT26 (colon carcinoma) 2000 c/w; HOS (osteosarcoma) 2500 c/w; ES-2 (ovarian carcinoma), HCT-116 (colorectal carcinoma), A-549 (lung carcinoma), A-172 (glioblastoma), D54 (glioblastoma), U87 (glioblastoma), T98G (glioblastoma), SK-ML-28 (melanoma), MCF-7 (breast cancer), AM38 (glioblastoma) and MDA-MB-231 (breast cancer) 3000 c/w; U118MG (malignant glioma) 4000 c/w; HepG2 (liver cancer) 8000 c/w. c/w=cells per well. IC₅₀ and Eₘₐₓ values shown in Table 6 were extracted from the raw data plotted in FIG. 8.

### DETAILED DESCRIPTION

Pantoea ananatis is the primary cause of onion center rot. Genetic data suggest that a phosphonic acid natural product is required for pathogenesis; however, the nature of the molecule is unknown. Here we show that P. ananatis produces at least three phosphonates, two of which were purified and structurally characterized. The first, designated pantaphos, was shown to be 2-(hydroxy(phosphono)methyl)maleate; the second, a probable biosynthetic precursor, was shown to be 2-(phosphonomethyl)maleate. Purified pantaphos is both necessary and sufficient for the hallmark lesions of onion center rot. Moreover, when tested against mustard seedlings, the phytotoxic activity of pantaphos was comparable to the widely used herbicides glyphosate and phosphinothricin. Pantaphos was also active against a variety of human cell lines but was significantly more toxic to glioblastoma cells. Pantaphos showed little activity when tested against a variety of bacteria and fungi.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, +10%, +20%, or +25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. The disease, disorder, and/or condition is in a living organism such as in an animal, plant, or crop. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

Compounds disclosed herein may be used as an active ingredient in a medicament to treat an animal or an active ingredient in an herbicidal formulation to treat a plant or crop. The active ingredient in the medicament or the herbicide is administered in an amount that is effective for the treatment of a disease, disorder, or condition in the animal, plant, or crop.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs or herbicides.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobuty)), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidazolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R‘ wherein each R’ can independently be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “IC₅₀” is generally defined as the concentration required to kill 50% of the cells in 24 hours.

The term “genome” or “genomic DNA” refers to the heritable genetic information of a host organism. Said genomic DNA comprises the entire genetic material of a cell or an organism, including the DNA of the bacterial chromosome and plasmids for prokaryotic organisms and includes for eukaryotic organisms the DNA of the nucleus (chromosomal DNA), extrachromosomal DNA, and organellar DNA (e.g., of mitochondria). Preferably, the terms genome or genomic DNA refer to the chromosomal DNA of the nucleus.

The term “chromosomal DNA” or “chromosomal DNA sequence” in the context of eukaryotic cells is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), in situ PCR and next generation sequencing (NGS).

The term “promoter” refers to a polynucleotide which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent if the promoter is a constitutive promoter. The term “enhancer” refers to a polynucleotide. An enhancer can increase the efficiency with which a particular gene is transcribed into mRNA irrespective of the distance or orientation of the enhancer relative to the start site of transcription. Usually, an enhancer is located close to a promoter, a 5′-untranslated sequence or in an intron.

A polynucleotide is “heterologous to” an organism or a second polynucleotide if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e. g. a genetically engineered coding sequence or an allele from a different ecotype or variety).

“Transgene”, “transgenic” or “recombinant” refers to a polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning-A Laboratory Manual, Cold Spring Harbor Laboratory, Cold Spring Harbor, New York, (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill in the art will recognize that polynucleotides can be manipulated in many ways and are not limited to the examples above.

In case the term “recombinant” is used to specify an organism or cell, e.g., a microorganism, it is used to express that the organism or cell comprises at least one “transgene”, “transgenic” or “recombinant” polynucleotide, which is usually the specified.

A polynucleotide “exogenous to” an individual organism is a polynucleotide which is introduced into the organism by any means other than by a sexual cross.

The terms “operable linkage” or “operably linked” are generally understood as meaning an arrangement in which a genetic control sequence, e.g., a promoter, enhancer or terminator, is capable of exerting its function with regard to a polynucleotide being operably linked to it, for example a polynucleotide encoding a polypeptide. Function, in this context, may mean for example control of the expression, i.e., transcription and/or translation, of the nucleic acid sequence. Control, in this context, encompasses for example initiating, increasing, governing, or suppressing the expression, i.e., transcription and, if appropriate, translation. Controlling, in turn, may be, for example, tissue- and/or time-specific. It may also be inducible, for example by certain chemicals, stress, pathogens, and the like. Preferably, operable linkage is understood as meaning for example the sequential arrangement of a promoter, of the nucleic acid sequence to be expressed and, if appropriate, further regulatory elements such as, for example, a terminator, in such a way that each of the regulatory elements can fulfill its function when the nucleic acid sequence is expressed. An operably linkage does not necessarily require a direct linkage in the chemical sense. For example, genetic control sequences like enhancer sequences are also capable of exerting their function on the target sequence from positions located at a distance to the polynucleotide, which is operably linked. Preferred arrangements are those in which the nucleic acid sequence to be expressed is positioned after a sequence acting as promoter so that the two sequences are linked covalently to one another. The distance between the promoter and the amino acid sequence encoding polynucleotide in an expression cassette, is preferably less than 200 base pairs, especially preferably less than 100 base pairs, very especially preferably less than 50 base pairs. The skilled worker is familiar with a variety of ways in order to obtain such an expression cassette. However, an expression cassette may also be constructed in such a way that the nucleic acid sequence to be expressed is brought under the control of an endogenous genetic control element, for example an endogenous promoter, for example by means of homologous recombination or else by random insertion. Such constructs are likewise understood as being expression cassettes for the purposes of the invention.

The term “expression cassette” or “expression vector” means those constructs in which the nucleic acid sequence encoding an amino acid sequence to be expressed is linked operably to at least one genetic control element which enables or regulates its expression (i.e., transcription and/or translation). The expression may be, for example, stable or transient, constitutive, or inducible. Examples of expression vectors are well known in the art and are described, for example, in U.S. Pat. No. 7,416,874.

The terms “express,” “expressing,” “expressed” and “expression” refer to expression of a gene product (e.g., a biosynthetic enzyme of a gene of a pathway or reaction defined and described in this application) at a level that the resulting enzyme activity of this protein encoded for or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

In some embodiments, a microorganism can be physically or environmentally altered to express a gene product at an increased or lower level relative to level of expression of the gene product unaltered microorganism. For example, a microorganism can be treated with, or cultured in the presence of an agent known, or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

The term “vector”, preferably, encompasses phage, plasmid, fosmid, viral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a recombinant microorganism. The vector may be incorporated into a recombinant microorganism by various techniques well known in the art. If introduced into a recombinant microorganism, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques.

The terms “transformation” and “transfection”, conjugation and transduction, as used in the present context, are intended to comprise a multiplicity of prior-art processes for introducing foreign nucleic acid (for example DNA) into a recombinant microorganism, including calcium phosphate, rubidium chloride or calcium chloride co-precipitation, DEAE-dextran-mediated transfection, lipofection, natural competence, carbon-based clusters, chemically mediated transfer, electroporation or particle bombardment. Methods for many species of microorganisms are readily available in the literature.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

The term “sequence identity” between two nucleic acid sequences is understood as meaning the percent identity of the nucleic acid sequence over in each case the entire sequence length which is calculated by alignment with the aid of the program algorithm GAP (Wisconsin Package

Version 10.0, University of Wisconsin, Genetics Computer Group (GCG), Madison, USA), setting, for example, the following parameters:

Gap Weight: 12 Length Weight: 4; Average Match: 2,912 Average Mismatch: −2,003.

The term “sequence identity” between two amino acid sequences is understood as meaning the percent identity of the amino acids sequence over in each case the entire sequence length which is calculated by alignment with the aid of the program algorithm GAP (Wisconsin Package Version 10.0, University of Wisconsin, Genetics Computer Group (GCG), Madison, USA), setting, for example, the following parameters: Gap Weight: 8; Length Weight: 2; Average Match: 2,912; Average Mismatch: −2,003.

The term “hybridization” as defined herein is a process wherein substantially homologous complementary nucleotide sequences anneal to each other. The hybridization process can occur entirely in solution, i.e., both complementary nucleic acids are in solution. The hybridization process can also occur with one of the complementary nucleic acids immobilized to a matrix such as magnetic beads, Sepharose beads or any other resin. The hybridization process can furthermore occur with one of the complementary nucleic acids immobilized to a solid support such as a nitro-cellulose or nylon membrane or immobilized by e.g., photolithography to, for example, a siliceous glass support (the latter known as nucleic acid arrays or microarrays or as nucleic acid chips). In order to allow hybridization to occur, the nucleic acid molecules are generally thermally or chemically denatured to melt a double strand into two single strands and/or to remove hairpins or other secondary structures from single stranded nucleic acids.

The term “stringency” refers to the conditions under which a hybridization takes place. The stringency of hybridization is influenced by conditions such as temperature, salt concentration, ionic strength, and hybridization buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridization conditions are typically used for isolating hybridizing sequences that have high sequence similarity to the target nucleic acid sequence. However, nucleic acids may deviate in sequence and still encode a substantially identical polypeptide, due to the degeneracy of the genetic code. Therefore, medium stringency hybridization conditions may sometimes be needed to identify such nucleic acid molecules.

The Tₘ is the temperature under defined ionic strength and pH, at which 50% of the target sequence hybridizes to a perfectly matched probe. The Tₘ is dependent upon the solution conditions and the base composition and length of the probe. For example, longer sequences hybridize specifically at higher temperatures. The maximum rate of hybridization is obtained from about 16° C. up to 32° C. below Tₘ. The presence of monovalent cations in the hybridization solution reduces the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridization to be performed at 30 to 45° C., though the rate of hybridization will be lowered. Base pair mismatches reduce the hybridization rate and the thermal stability of the duplexes. On average and for large probes, the Tₘ decreases about 1° C. per % base mismatch. The Tₘ may be calculated using the following equations, depending on the types of hybrids:
1) DNA-DNA hybrids (Meinkoth and Wahl, Anal. Biochem., 138: 267-284, 1984):

Tₘ81.5° C.+16.6×log io[Na⁺]^{a}+0.41×%[G/C^{b}]−500×[L^{c}]−¹−0.61×% formamide
2) DNA-RNA or RNA-RNA hybrids:

Tₘ=79.8° C.+18.5(log io[Na⁺]^{a})+0.58(% G/C^{b})+11.8(% G/C^{b})²−820/L^{c}

3) oligo-DNA or oligo-RNA^{d} hybrids:
For <20 nucleotides: Tₘ 2 (lₙ)
For 20-35 nucleotides: Tₘ=22+1 0.46 (lₙ)
a or for other monovalent cation, but only accurate in the 0.01-0.4 M range.
b only accurate for % GC in the 30% to 75% range.
c L=length of duplex in base pairs.
d oligo, oligonucleotide; lₙ, =effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridization buffer, and treatment with RNAse. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridization, and which will either maintain or change the stringency conditions.

Besides the hybridization conditions, specificity of hybridization typically also depends on the function of post-hybridization washes. To remove background resulting from non-specific hybridization, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridization stringency. A positive hybridization gives a signal that is at least twice of that of the background. Generally, suitable stringent conditions for nucleic acid hybridization assays or gene amplification detection procedures are as set forth above. More or less stringent conditions may also be selected. The skilled artisan is aware of various parameters which may be altered during washing, and which will either maintain or change the stringency conditions.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. The length of the hybrid is the anticipated length for the hybridizing nucleic acid. When nucleic acids of known sequence are hybridized, the hybrid length may be determined by aligning the sequences and identifying the conserved regions described herein. 1×SSC is 0.15M NaCl and 15 mM sodium citrate; the hybridization solution and wash solutions may additionally include 5×Denhardt's reagent, 0.5-1.0% SDS, 100 g/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Embodiments of the Technology

This disclosure provides a composition comprising a compound of Formula I:

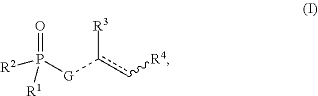

or a salt thereof; wherein
   [symbol] represents a single or double bond; [symbol] represents a double or single bond, wherein both [symbol] and [symbol] are not double bonds;
   G is X^{A}CHOR⁵, O, C(═O), C(═CH₂), CHP(═O)(R⁶)₂, or CX^{B}₂;
   X^{A} is absent or O; each X^{B} is independently H or halo;
   R¹ and R² are each independently OR^{A} or an amino acid;
   R³ is —C(═O)R⁷ or a triazole or tetrazole;
   R⁴ is —C(═O)R⁸ or a triazole or tetrazole;
   R⁵ is H, —(C₁-C₆)alkyl, —(C₃-C₆)cycloalkyl, aryl, or heteroaryl;
   each R⁶ is independently OR^{B} or an amino acid;
   R⁷ and R⁸ are each independently OR^{C} or an amino acid; and
   each R^{A}, R^{B} and R^{C} are independently H, —(C₁-C₆)alkyl, —(C₃-C₆)cycloalkyl, aryl, or heteroaryl; and
a fluid (aqueous or non-aqueous), additive (non-naturally occurring) or combination thereof.

In various embodiments, R¹, R², R⁶, R⁷ and R⁸ are each independently NR^{X}R^{Y}, wherein each R^{X} and R^{Y} are independently H, —(C₁-C₆)alkyl, —(C₃-C₆)cycloalkyl, aryl, or heteroaryl. In various additional embodiments, R³ and R⁴ are each independently NO₂, CO₂R^{X}, P(═O)(OR^{X})₂, S(═O)₂OR^{X}, or S(═O)R^{X}, wherein each R^{X} is independently H, —(C₁-C₆)alkyl, or —(C₁-C₆)cycloalkyl. In various other embodiments, the compound is a prodrug. In various embodiments, the compound is suitably substituted with a substituent (e.g., to form an ester) that is metabolized or cleaved to release the active form of the compound (e.g., pantaphos).

In some embodiments, the fluid is water or an aqueous solution, a non-aqueous fluid or solution, an oil, an organic solvent, a liquid, or combination thereof. In other embodiments, the composition is formulated as a powder, fine powder, granule, or pellet. The formulation can comprise additives, salts, an emulsifier, nanoparticles, surfactants, buffering agents, wetting agents, colloids, lipids, phospholipids, biodegradable polymers, a second active agent, one or more active agents, or a combination thereof. In other embodiments, the compound in the composition can be encapsulated in a micro- or nanocapsule, or a time release capsule.

In various embodiments, G is CHOR⁵. In various embodiments, the compound is the (S)-enantiomer. In various embodiments, the compound is the (R)-enantiomer. In various embodiments, R¹ and R² are OR^{A}. In various embodiments, R³ and R⁴ are —CO₂R^{C}. In various embodiments, R³ and R⁴ have a cis-configuration when [symbol] is double bond.

In various embodiments, a compound of Formula I is represented by Formula II.

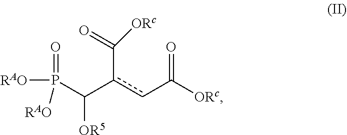

or a salt thereof.

In various embodiments, the compound is pantaphos:

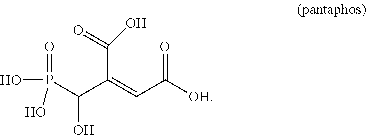 [SMILES: O=C(O)/C=C(\C(=O)O)C(O)P(=O)(O)O]

In various embodiments, the compound is compound 2:

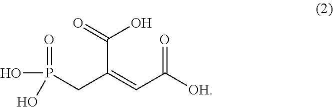 [SMILES: O=C(O)/C=C(/CP(=O)(O)O)C(=O)O]

In some embodiments, the composition comprises pantaphos and compound 2.

This disclosure also provides a compound of Formula I:

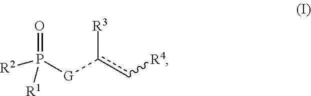

or a salt thereof, wherein
[symbol] represents double or single bond;
[symbol] represents double or single bond, wherein both [symbol] and [symbol] are not double bonds;
   G is X^{A}CHOR⁵, O, C(═O), C(═CH₂), CHP(═O)(R⁶)₂, or CX^{B}₂;
   X^{A} is absent or O; each X^{B} is independently H or halo;
   R¹ and R² are each independently OR^{A} or an amino acid;
   R³ is —C(═O)R⁷ or a triazole or tetrazole;
   R⁴ is —C(═O)R⁸ or a triazole or tetrazole;
   R⁵ is H, —(C₁-C₆)alkyl, —(C₃-C₆)cycloalkyl, aryl, or heteroaryl;
   each R⁶ is independently OR^{B} or an amino acid;
   R⁷ and R⁸ are each independently OR^{C} or an amino acid; and
   each R^{A}, R^{B} and R^{C} are independently H, —(C₁-C₆)alkyl, —(C₃-C₆)cycloalkyl, aryl, or heteroaryl.

In some embodiments, the compound is not a natural product. In some embodiments, the compound is not 2-(hydroxy(phosphono)methyl)maleic acid or 2-(phosphonomethyl) maleic acid. In various embodiments, G is CHOH. In various embodiments, R¹ and R² are OH. In various embodiments, R³ and R⁴ are —CO₂H. In other embodiments the compound is represented by any one of the following structures:

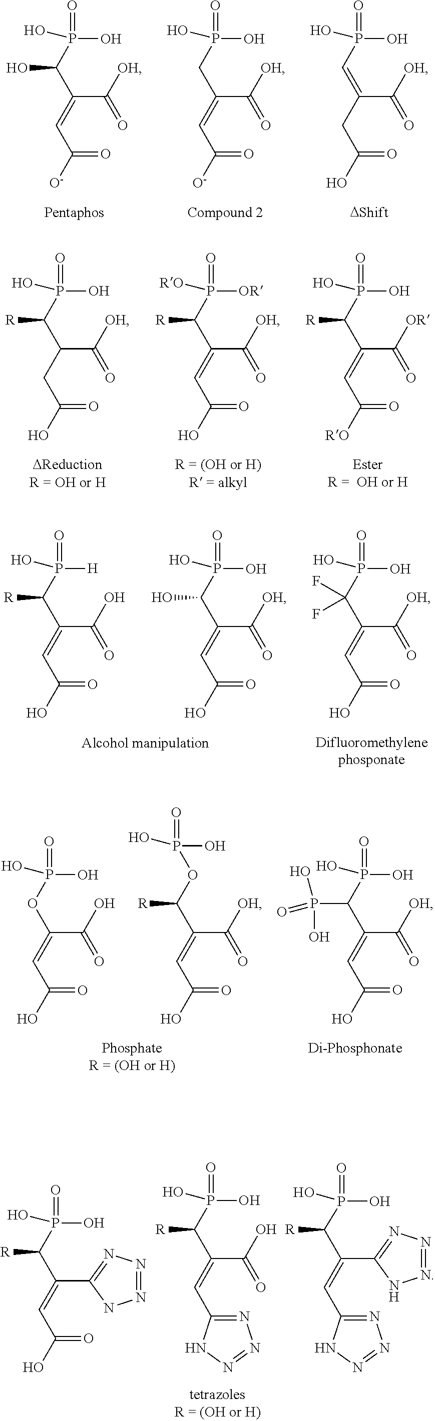

In some embodiments the compound is represented by:

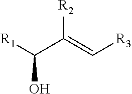

or the enantiomer thereof, wherein R₁, R₂, and R₃ are each independently carboxylate, phosphonate, nitrate, sulfonate, sulfoxide, or a combination thereof;

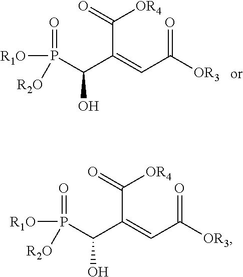

wherein R¹, R², R³, and R⁴ are each independently H, alkyl, an aromatic group, or a combination thereof;

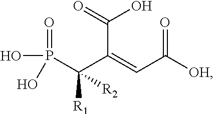

wherein R¹ and R² are each independently H, F, Cl, Br, I, or a combination thereof;

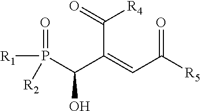

or the enantiomer thereof,
wherein R¹, R², R⁴, and R⁵ are each independently any amino acid, wherein the amino acid forms an amide or phosphonamide linkage.

In some embodiments, an aqueous composition comprises any one or more of the structures disclosed herein. In some embodiments the composition comprises adjuvants and surfactants known to one of ordinary skill in the art for herbicidal formulations. In various embodiments, the compound is the (R)- or (S)-enantiomer. In various embodiments, the compound is levorotatory or dextrorotatory. In various embodiments, the compound is a salt.

Also, this disclosure provides a method for inhibiting growth or formation of a weed comprising contacting the weed and/or soil where the weed can form and a herbicidally effective amount of a composition or compound disclosed herein, wherein growth or formation of the weed is inhibited. In some embodiments, the weed is killed. In some embodiments, the weed is killed, or blocked or suppressed from germinating without significantly harming other plants, vegetation or crops.

In some embodiments, the weed is controlled, where control is the destruction of unwanted weeds, or the damage of them to the point where they are no longer competitive with a crop, other plants or vegetation. In some other embodiments, the weed is suppressed, where suppression is incomplete control but provides an economic benefit, such as reduced competition with a crop, other plants or vegetation.

In some embodiments, the composition or compound contacts vegetation and/or soil where the vegetation can form, and growth or formation of the weed is selectively inhibited.

Additionally, this disclosure provides a method for inhibiting growth of a cancer cell comprising contacting the cancer cell and an effective amount of a composition or compound disclosed herein, wherein growth of the cancer is inhibited. In some embodiments, the cancer cell is a glioblastoma cell.

Furthermore, this disclosure provides a method for treating cancer in a subject in need thereof comprising administering a therapeutically effective amount of the compound or composition disclosed herein, wherein the cancer is treated.

In various embodiments, an effective amount of a compound disclosed herein, such as pantaphos, is about 0.01 mg/m² or less to about 100 g/m² or more in for an animal or crop. In other embodiments, the effective amount is about 0.1 mg/m² to about 10 g/m². In other embodiments, the effective amount is about 0.05 mg/m², 0.1 mg/m², 0.2 mg/m², about 0.5 mg/m², 1 mg/m², about 2 mg/m², about 5 mg/m², 10 mg/m², about 15 mg/m², 20 mg/m², about 50 mg/m², about 100 mg/m², 200 mg/m², about 300 mg/m², 500 mg/m², about 750 mg/m², about 1000 mg/m², 1500 mg/m², about 2000 mg/m², 3000 mg/m², about 5000 mg/m², about 7500 mg/m², 10,000 mg/m², about 20,000 mg/m², 50,000 mg/m², about 75,000 mg/m², about 100,000 mg/m², or any amount between the cited amounts.

In various embodiments, a compound disclosed herein in an herbicidal composition or pharmaceutical composition is synergistic with a second active agent to control weeds or treat a cancer.

Furthermore, this disclosure provides a method for forming 2-(hydroxy(phosphono)methyl)maleic acid:

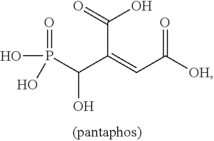 [SMILES: O=C(O)/C=C(\C(=O)O)C(O)P(=O)(O)O]

or salt thereof, comprising:
   a) isomerizing phosphoenolpyruvate (PEP; 2-(phosphonooxy)acrylic acid) to 3-phosphonopyruvate (PnPy; 2-oxo-3-phosphonopropanoic acid);
   b) condensing an acetyl group and PnPy to form phosphonomethylmalate (PMM; 2-hydroxy-2-(phosphonomethyl)succinic acid);
   c) dehydrating PMM to 2-phosphonomethylmaleate (2-(phosphonomethyl)maleic acid); and
   d) oxidizing 2-phosphonomethylmaleate to pantaphos (2-(hydroxy(phosphono)methyl) maleic acid);
wherein each step a)-d) is completed in in-vitro, a vessel, or reactor, wherein the vessel or reactor is man-made.

In some embodiments, isomerizing is catalyzed by PEP mutase (HvrA); condensing is catalyzed by phosphonomethylmalate synthase (HvrC) and the acetyl group is acetyl-CoA; dehydrating is catalyzed by large isopropylmalate dehydratase (HvrD) and/or small isopropylmalate dehydratase (HvrE) dehydratase; and oxidizing is catalyzed by flavin-dependent monooxygenase (HvrB) and optionally flavin reductase (HvrK).

In some embodiments, the compound pantaphos can be prepared from a sequential biosynthetic process using purified enzymes HvrA, HvrC, HvrDE and HvrBK (see Scheme 1), in the order shown, beginning by contacting phosphoenolpyruvate (PEP) and HvrA, wherein the reaction products are contacted by the next enzyme in the sequence.

In another embodiment, the compound pantaphos can be prepared from a sequential biosynthetic process using purified enzymes HvrA (SEQ ID NO: 14), HvrC (SEQ ID NO: 16), HvrD (SEQ ID NO: 17), HvrE (SEQ ID NO: 18), HvrB (SEQ ID NO: 15), and HvrK (SEQ ID NO: 24) (see Scheme 1), in the order shown, beginning by contacting phosphoenolpyruvate (PEP) and HvrA (SEQ ID NO: 14), wherein the reaction products are contacted by the next enzyme in the sequence.

Additionally, this disclosure provides a nucleic acid molecule comprising an hvr operon (hvrA-hvrL) of Pantoea Sp. In other embodiments, a nucleic acid molecule comprises one or more genes selected from the group consisting of hvrA, hvrB, hvrC, hvrD, hvrE, and hvrK of Pantoea Sp. In another embodiment, a nucleic acid molecule comprises the genes hvrA, hvrB, hvrC, hvrD, hvrE, and hvrK of Pantoea Sp.

In other embodiments, a nucleic acid molecule comprises the genes hvrA (SEQ ID NO: 1), hvrB (SEQ ID NO: 2), hvrC (SEQ ID NO: 3), hvrD (SEQ ID NO: 4), hvrE (SEQ ID NO: 5), hvrF (SEQ ID NO: 6), hvrG (SEQ ID NO: 7), hvrH (SEQ ID NO: 8), hvrI (SEQ ID NO: 9), hvrJ (SEQ ID NO: 10), hvrK (SEQ ID NO: 11), and hvrL (SEQ ID NO: 12) of Pantoea ananatis.

In some embodiments, a nucleic acid molecule comprises one or more genes selected from the group consisting of hvrA (SEQ ID NO: 1), hvrB (SEQ ID NO: 2), hvrC (SEQ ID NO: 3), hvrD (SEQ ID NO: 4), hvrE (SEQ ID NO: 5), hvrF (SEQ ID NO: 6), hvrG (SEQ ID NO: 7), hvrH (SEQ ID NO: 8), hvrI (SEQ ID NO: 9), hvrJ (SEQ ID NO: 10), hvrK (SEQ ID NO: 11), and hvrL (SEQ ID NO: 12) of Pantoea ananatis.

In other embodiments, a nucleic acid molecule comprises the genes hvrA (SEQ ID NO: 1), hvrB (SEQ ID NO: 2), hvrD (SEQ ID NO: 4), hvrE (SEQ ID NO: 5), and hvrK (SEQ ID NO: 11) of Pantoea ananatis. In still other embodiments, a nucleic acid molecule comprises the genes hvrA (SEQ ID NO: 1), hvrB (SEQ ID NO: 2), hvrC (SEQ ID NO: 3), hvrD (SEQ ID NO: 4), hvrE (SEQ ID NO: 5), and hvrK (SEQ ID NO: 11) of Pantoea ananatis.

In other embodiments, a nucleic acid molecule comprises one or more genes selected from the group consisting of hvrA (SEQ ID NO: 1), hvrB (SEQ ID NO: 2), hvrC (SEQ ID NO: 3), hvrD (SEQ ID NO: 4), hvrE (SEQ ID NO: 5), and hvrK (SEQ ID NO: 11) of Pantoea ananatis.

In other embodiments, the disclosure provides a nucleic acid molecule comprising an hvr operon (hvrA-hvrL) of Pantoea Sp. operably linked to an inducible promotor sequence, wherein induction of the promoter and expression of the genes of the hvr operon causes the production of a phosphonate compound of Formula I or Formula II. In various embodiments the phosphonate compound is 2-(hydroxy(phosphono)methyl) maleic acid (pantaphos).

Suitable inducible promoters for use with various embodiments include, but are not limited to, T7, T7iac, rpoS, PrhaBAD, mmsA, trc, tetA, tac, lac, tacM, P_{L}, araBAD, cspA, cspB, phyL, NBP3510, P43, Pspac, P₁₇₀, Pgrac, and trp. In some embodiments, the inducible promoter is a lac or a tac promoter. In some embodiments, the inducible promoter is a tac promoter having the nucleic acid sequence according to SEQ ID NO: 13.

In other embodiments, a nucleic acid molecule comprises an inducible promoter and one or more genes selected from the group consisting of hvrA, hvrB, hvrC, hvrD, hvrE, and hvrK of Pantoea Sp., wherein the one or more genes are operably linked to the inducible promoter.

In some embodiments, a nucleic acid molecule comprises an inducible promoter and the genes hvrA (SEQ ID NO: 1), hvrB (SEQ ID NO: 2), hvrC (SEQ ID NO: 3), hvrD (SEQ ID NO: 4), hvrE (SEQ ID NO: 5), hvrF (SEQ ID NO: 6), hvrG (SEQ ID NO: 7), hvrH (SEQ ID NO: 8), hvrI (SEQ ID NO: 9), hvrJ (SEQ ID NO: 10), hvrK (SEQ ID NO: 11), and hvrL (SEQ ID NO: 12) of Pantoea ananatis, wherein the genes are operably linked to the inducible promoter.

In some embodiments, a nucleic acid molecule comprises an inducible promoter and one or more genes selected from the group consisting of hvrA (SEQ ID NO: 1), hvrB (SEQ ID NO: 2), hvrC (SEQ ID NO: 3), hvrD (SEQ ID NO: 4), hvrE (SEQ ID NO: 5), hvrF (SEQ ID NO: 6), hvrG (SEQ ID NO: 7), hvrH (SEQ ID NO: 8), hvrI (SEQ ID NO: 9), hvrJ (SEQ ID NO: 10), hvrK (SEQ ID NO: 11), and hvrL (SEQ ID NO: 12) of Pantoea ananatis, wherein the one or more genes are operably linked to the inducible promoter.

In other embodiments, a nucleic acid molecule comprises an inducible promoter and the genes hvrA (SEQ ID NO: 1), hvrB (SEQ ID NO: 2) hvrD (SEQ ID NO: 4), hvrE (SEQ ID NO: 5), and hvrK (SEQ ID NO: 11) of Pantoea ananatis, wherein the genes are operably linked to the inducible promoter. In another embodiment, a nucleic acid molecule comprises an inducible promoter and the genes hvrA (SEQ ID NO: 1), hvrB (SEQ ID NO: 2), hvrC (SEQ ID NO: 3), hvrD (SEQ ID NO: 4), hvrE (SEQ ID NO: 5), and hvrK (SEQ ID NO: 11) of Pantoea ananatis, wherein the genes are operably linked to the inducible promoter.

In other embodiments, a nucleic acid molecule comprises an inducible promoter and one or more genes selected from the group consisting of hvrA (SEQ ID NO: 1), hvrB (SEQ ID NO: 2), hvrD (SEQ ID NO: 4), hvrE (SEQ ID NO: 5), and hvrK (SEQ ID NO: 11) of Pantoea ananatis or one or more genes selected from the group consisting of hvrA (SEQ ID NO: 1), hvrB (SEQ ID NO: 2), ), hvrC (SEQ ID NO: 3), hvrD (SEQ ID NO: 4), hvrE (SEQ ID NO: 5), and hvrK (SEQ ID NO: 11) of Pantoea ananatis.

In another embodiment, a nucleic acid molecule comprises an inducible promoter according to the nucleic acid sequence of SEQ ID NO: 13 and a nucleic acid sequence according to SEQ ID NO: 1, 2, 3, 4, 5, and 11 encoding the genes hvrA, hvrB, hvrC, hvrD, hvrE, and hvrK, respectively, wherein the genes are operably linked to the inducible promoter.

In some embodiments, a nucleic acid molecule comprises a nucleic acid sequence having at least 60%, preferably at least 65, 70, 75, 80, 85, 90, 95, 96, 97, 98 or 99%, identity to SEQ ID NO: 1, 2, 3, 4, 5, 6, 7, 8, 9, 10, 11, and 12.

Also, this disclosure provides a recombinant cell for producing a phosphonate compound according to Formula I or Formula II comprising a nucleic acid molecule (e.g., expression vector) disclosed herein. In some embodiments, a recombinant cell expresses one or more proteins selected from the group consisting of SEQ ID NO: 14, 15, 16, 17, 18, 19, 20, 21, 22, 23, 24, and 25. In some embodiments, a recombinant cell expresses one or more proteins having an amino acid sequence having at least 60%, preferably at least 65, 70, 75, 80, 85, 90, 95, 96, 97, 98 or 99%, identity to one or more proteins selected from the group consisting of SEQ ID NO: 14, 15, 16, 17, 18, 19, 20, 21, 22, 23, 24, and 25.

In various embodiments, the nucleic acid molecule is inserted into a cell and maintained as a plasmid or integrated into a chromosome of the cell. In various embodiments, the cell is selected from a genus from the group consisting of Pantoea, Clostridium, Zymomonas, Escherichia, Salmonella, Serratia, Erwinia, Klebsiella, Shigella, Rhodococcus, Pseudomonas, Bacillus, Lactobacillus, Lactococcus, Enterococcus, Alcaligenes, Paenibacillus, Arthrobacter, Corynebacterium, Brevibacterium, Schizosaccharomyces, Kluveromyces, Yarrowia, Pichia, Zygosaccharomyces, Debaryomyces, Candida, Brettanomyces, Pachysolen, Hansenula, Issatchenkia, Trichosporon, Yamadazyma, and Saccharomyces.

In various embodiments, the cell is of the genus Pantoea, Escherichia, or Saccharomyces. Examples of species belonging to the Pantoea genus include, but are not limited to Pantoea agglomerans, Pantoea ananatis, Pantoea stewartii, Pantoea citrea, Pantoea dispersa, Pantoea punctate, Pantoea terrea, Pantoea deleyi, Pantoea anthophila, Pantoea allii, and Pantoea eucalypti. In various embodiments, the cell is Pantoea ananatis, Escherichia coli, or Saccharomyces cerevisiae.

Methods of making recombinant cells and nucleic acid molecules are known in the art, are described herein and, for example, in International Publication Nos. WO 2020/090940 and WO 2014/129898, and in U.S. Publication Nos. 2019/0256838 and 2014/0134689.

This disclosure also provides a process for producing a phosphonate compound according to Formula I or Formula II comprising the steps of:
a) providing a cell culture of a recombinant cell disclosed herein (see above), wherein the recombinant cell produces the phosphonate, and the cell culture is about 1 L to about 10 L in volume;
b) mixing an inducer molecule with the recombinant cell culture;
c) incubating the induced recombinant cell culture for up to 96 hours with constant oxygenation;
d) pelleting cells of the recombinant cell culture and collecting a supernatant;
e) concentrating the supernatant;
f) extracting the phosphonate from the concentrated supernatant using methanol extraction to form an extracted supernatant; and
g) purifying the phosphonate from a methanol soluble fraction of the extracted supernatant.

In various embodiments, the phosphonate is 2-(hydroxy(phosphono)methyl)maleic acid. In some embodiments, step g comprises iron-IMAC purification followed by flash chromatography and HILIC HPLC. In some embodiments, the cell culture comprises a recombinant Pantoea ananatis, Escherichia coli, or Saccharomyces cerevisiae cell. In some embodiments, the cell culture is a recombinant Pantoea ananatis. In some embodiments, the constant oxygenation has a flow rate of 5 L/min, and the cell culture is maintained at a temperature of 30° C.

Another embodiment of the present disclosure provides a compound comprising a hydroxylated allyl phosphonic acid of Formula A, wherein R₁ and R₂ are independently selected from the group comprising carboxylic acids and derivatives (ketones, esters, carboxylic acids); hydroxyl groups; amines; ethers; halogens; alkyl or aryl groups; or alkyl or aryl groups containing the abovementioned functional groups.

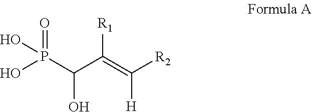

Another embodiment of the present disclosure provides a compound comprising a hydroxylated allyl phosphonic acid of Formula B, wherein each R is independently selected from the group comprising halogens, amino acids, carboxylic acids and derivatives (ketones, esters, carboxylic acids); hydroxyl groups; amines; ethers; halogens; alkyl or aryl groups; or alkyl or aryl groups containing the abovementioned functional groups.

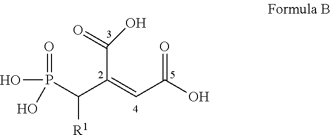

Another embodiment of the present disclosure provides a compound of Formula A wherein R₁ and R₂ are in the (Z) configuration.

Another embodiment of the present disclosure provides a compound of Formula A wherein R₁ and R₂ are in the (E) configuration.

Another embodiment of the present disclosure provides a compound of Formula A or Formula B wherein the chiral orientation is R.

Another embodiment of the present disclosure provides a compound of Formula A or Formula B wherein the chiral orientation is S.

Another embodiment of the present disclosure provides a compound of Formula A wherein R₁ and R₂ are COOH or COO—.

Another embodiment of the present disclosure provides a compound of Formula A wherein R₁ and R₂ are COOH or COO—, and one or both of the carboxylates is provided as a salt selected of the group comprising monovalent and divalent counterions.

Another embodiment of the present disclosure provides a salted form of the compound of Formula A and/or Formula B wherein the monovalent and divalent counterions are selected from the group consisting of sodium, potassium, calcium, lithium, magnesium, manganese, and mixtures thereof.

Another embodiment of the present disclosure provides a herbicidal formulation comprising the compound of Formula A and/or Formula B that is effective against plants.

Another embodiment of the present disclosure provides a salted form of the compound of Formula A and/or Formula B that is effective against monocot plants.

Another embodiment of the present disclosure provides a salted form of the compound of Formula A and/or Formula B that is effective against dicot plants.

Another embodiment of the present disclosure provides a salted form of the compound of Formula A and/or Formula B comprising water, one or more emulsifying agents, one or more surfactants, one or more wetting agents, and/or one or more pH buffering components, wherein the said composition has a pH between 5 and 9.

Another embodiment of the present disclosure provides a salted form of the compound of Formula A and/or Formula B comprising other bioactive agents, including other pesticides, adjuvants, or macro- or micro-nutrients.

Another embodiment of the present disclosure provides a method of biosynthesizing the compound of Formula A and/or Formula B using bacteria of the genus Pantoea.

Another embodiment of the present disclosure provides a method of biosynthesizing the compound of Formula A and/or Formula B using bacteria, wherein the bacterial strain is Pantoea ananatis.

Another embodiment of the present disclosure provides a method of biosynthesizing the compound of Formula A and/or Formula B using the bacteria Pantoea wherein the expression of the hvr gene cluster is modified.

Another embodiment of the present disclosure provides a method of biosynthesizing the compound of Formula A and/or Formula B using the bacteria Pantoea, which has been genetically modified to biosynthesize the compound of Formula A and/or Formula B in titers of >3 mg/L.

Another embodiment of the present disclosure provides a genetically modified bacterium wherein the expression of the hvr gene cluster has been modified to produce Formula A and/or Formula B.

Another embodiment of the present disclosure provides an herbicidal composition comprising the bacteria, bacterial-derived isolates, or supernatant of the bacteria of any of the foregoing embodiments that is effective against plants.

Another embodiment of the present disclosure provides an herbicidal composition of the foregoing embodiments that is effective against monocot plants.

Another embodiment of the present disclosure provides an herbicidal composition of the foregoing embodiments that is effective against dicot plants.

Another embodiment of the present disclosure provides a method for drying, killing, or suppressing a plant, which comprises contacting the plant with an herbicidal composition of the foregoing embodiments.

Another embodiment of the present disclosure provides a method of treating or preventing a fungal infection comprising the step of administering an antifungal amount of a compound or Formula A and/or Formula B to a patient in need thereof.

Another embodiment of the present disclosure provides a method of treating or preventing cancer comprising the step of administering a therapeutic amount of a compound or Formula A and/or Formula B to a patient in need thereof.

Another embodiment of the present disclosure provides a method of treating or preventing cancer comprising the step of administering a therapeutic amount of a compound or Formula A and/or Formula B to a patient in need thereof wherein the patient is a mammal and the cancer is glioblastoma.

A Phosphonate Natural Product Made by Pantoea ananatis is Necessary and Sufficient for the Hallmark Lesions of Onion Center Rot.

Although the indirect evidence for the involvement of a bioactive phosphonate in P. ananatis pathogenesis is strong, this has yet to be established. Here, we show that the hvr operon indeed encodes enzymes responsible for production of a small molecule phosphonate, which we show to be 2-(hydroxy(phosphono)methyl)maleate. The purified molecule, which we have designated pantaphos, has significant herbicidal activity and is able to produce the characteristic lesions of onion center rot in the absence of P. ananatis. Accordingly, this novel phosphonate natural product is both necessary and sufficient for onion rot pathogenesis. In addition, pathogenicity is enhanced in strains lacking phosphonate catabolism, suggesting that endogenous catabolism of pantaphos attenuates virulence.

### Results.

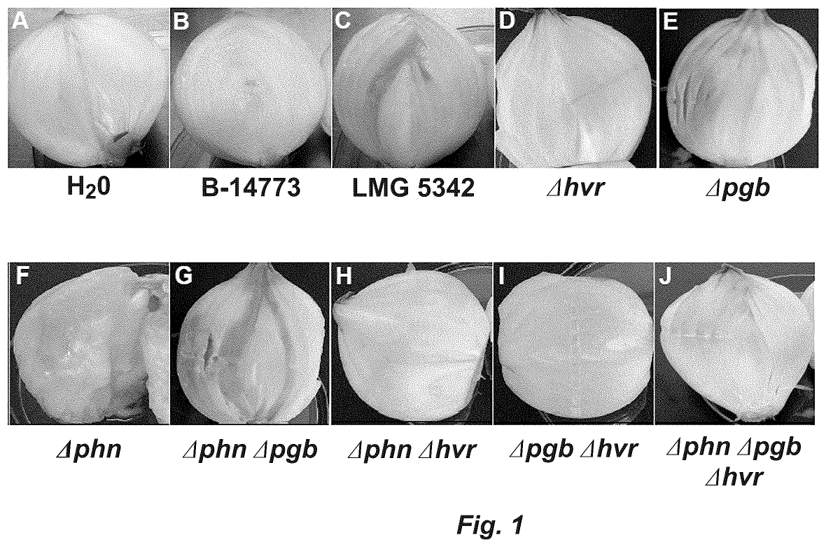

Role of phosphonate metabolism in onion center rot. To examine the broader role of phosphonate metabolism in onion pathogenesis, we characterized P. ananatis LMG 5342, which carries the hvr, pgb and phn loci, using P. ananatis B-14773, which does not encode any phosphonate biosynthetic genes, as a control. Because the pathogenicity of these strains has not been established, initial experiments were conducted to assess their ability to cause onion rot. Consistent with the previously observed correlation between the presence of the hvr locus and pathogenesis, we observed onion center rot in bulbs inoculated with strain LMG 5342, but not in bulbs inoculated with B-14773 (FIG. 1).

To verify that the observed pathogenic phenotype required hvr, and to examine whether the additional phosphonate metabolism genes play a role in pathogenesis, we constructed a series of LMG 5342 mutants lacking the hvr, pgb and phn loci in all possible combinations and scored their ability to cause onion rot (FIG. 1). The center rot phenotype was observed in all mutants that retained the hvr locus and absent in all strains with the Ahvr mutation. Therefore, as observed in P. ananatis OC5a, the hvr locus is necessary for onion pathogenicity in strain LMG 5342. In contrast, the pgb locus does not significantly contribute to pathogenicity, because the onion rot phenotype for mutants lacking these genes was identical to each of the otherwise isogenic strains. Interestingly, strains with intact hvr and phn loci were attenuated relative to those with the Δphn mutation, suggesting that endogenous phosphonate catabolism minimizes virulence (FIG. 1).

Production of phosphonic acids in P. ananatis LMG 5342. To examine whether phosphonates are actually produced by P. ananatis LMG 5342, we grew the wild-type strains in a variety of liquid and solid media. Spent media were then concentrated and screened for the presence of phosphonates using ³¹P nuclear magnetic resonance (NMR), which allows relatively sensitive detection of molecules containing a carbon-phosphorus (C—P) bond, even in complex mixtures containing phosphate and phosphate esters. In no case did we observe signals consistent with the presence of phosphonates. We suspected that our inability to detect phosphonates in spent media was due to poor expression of the hvr operon in the media we employed. Thus, we conducted similar experiments in media supplemented with onion extract, with the idea that a plant metabolite was required to induce expression of the hvr locus; however, we failed to detect phosphonates in these media as well.

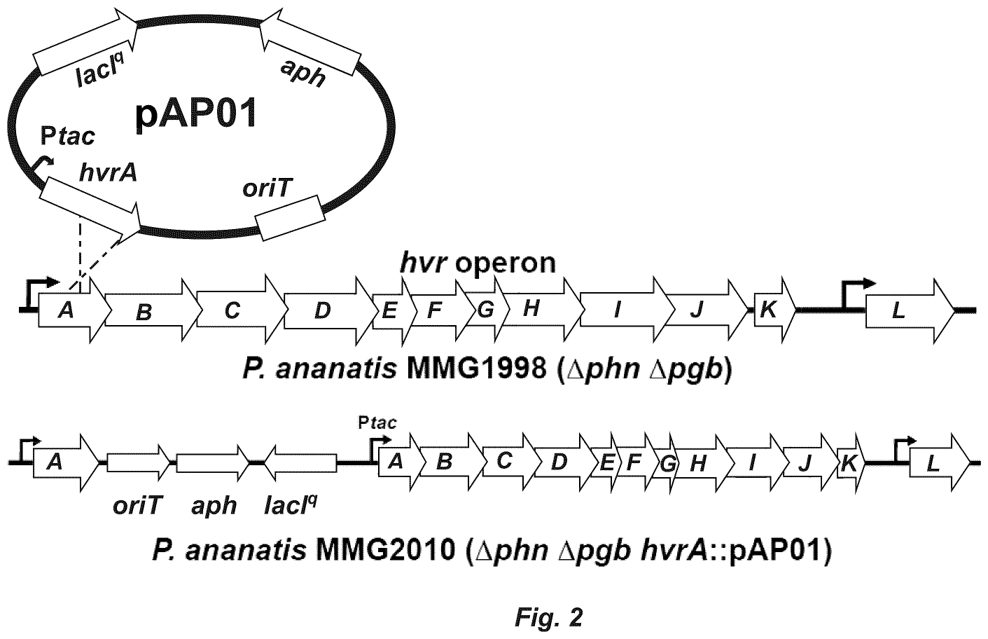

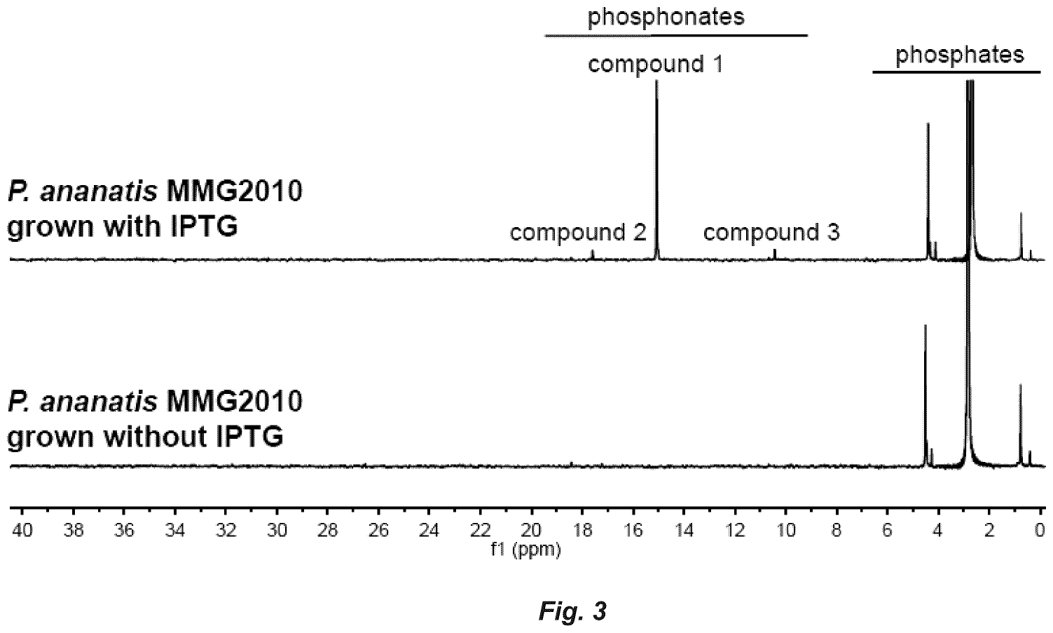

To circumvent issues arising from native gene regulation, we constructed a strain that expresses the hvr operon from a strong, isopropyl-β-D-1-thiogalactopyranoside (IPTG)-inducible promoter (FIG. 2). To avoid complications caused by the other phosphonate metabolic genes, this strain also carried the Δphn and Δpgb mutations. After growth of this recombinant strain in media with IPTG, three distinct ³¹P NMR peaks were observed (FIG. 3). The chemical shifts of these peaks (δ_{P} 17.6, δ 15.0, and δ 10.4 ppm) are consistent with the presence of molecules containing C—P bonds. These peaks were not observed after growth in media without IPTG. Thus, putative phosphonates are produced only when the hvr operon is expressed. After optimization of the medium and growth conditions, the IPTG-inducible strain catalyzed nearly complete conversion of phosphate to biomass and phosphonates, with final concentrations of approximately 0.653, 0.080, 0.039 mM for compounds 1; ³¹P NMR (δ 17.6), 2 (δ 15.0), and 3 (δ 10.4 ppm), respectively.

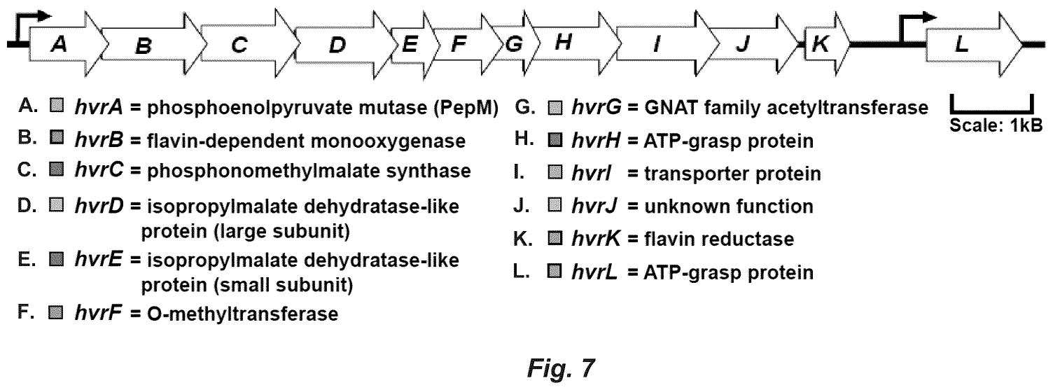

Structure elucidation of hvr-related phosphonates. From a 3.2-liter culture grown using these optimized conditions, we were able to isolate 8.9 mg of pure compound 1 and small amount (<600 μg) of pure compound 2. Compound 3 (δ_{P} 10.42 ppm) was not obtained, because it is unstable at the low pH used during affinity chromatography (data not shown). The structures of compounds 1 and 2 were elucidated using a series of one- and two-dimensional carbon, phosphorus and proton NMVR experiments (summarized in Table 1; the full data set is described in the Supplementary Dataset 2). Compound 1, which we designated pantaphos, was shown to be 2-(hydroxy(phosphono)methyl)maleate. Compound 2 (2-phosphonomethylmaleate) has a nearly identical structure, but lacks the hydroxyl-group, suggesting that it may be an intermediate in the biosynthesis of pantaphos (Table 1). High-resolution mass spectrometry data of the purified compounds are fully consistent with these structures (see Examples, FIG. 7).

TABLE 1
Summary of NMR data supporting the structure of compounds 1 and 2. Primary
data and full description of structural elucidation is provided in the Examples.

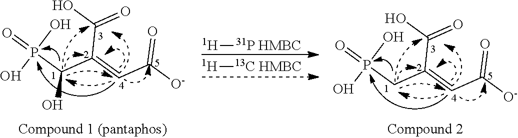

| | | | | | | |
|---|---|---|---|---|---|---|
| | δ_{C} | δ_{H} | ¹H-¹³C | ¹H-¹³C | ¹H-³¹P | ¹H-¹H |
| position | (mult.; J in Hz) | (mult.; J in Hz) | HSQC | HMBC | HMBC | NOSEY/COSY |

Compound 1 (pantaphos)

| | | | | | | |
|---|---|---|---|---|---|---|
| 1 | 71.00 | 4.31 | Y | H1→C2, | H1→δ_{P} | H4 |
| | (d; 144.0) | (d; 15.3) | | C3, C4 | 15.40 ppm | |
| 2 | 142.98 | | N | | | |
| 3 | 174.62 | | N | | | |
| 4 | 126.50 | 5.91 | Y | H4→C1, | H4→δ_{P} | H1 |
| | (d; 9.05) | (d; 6.00) | | C2, C3, C5 | 15.40 ppm | |
| 5 | 175.20 | | N | | | |

Compound 2

| | | | | | | |
|---|---|---|---|---|---|---|
| 1 | 34.05 | 2.43 | Y | H1→C2, | H1→δ_{P} | H4 |
| | (d; 123.0) | (d; 18.0) | | C3, C4 | 18.47 ppm | |
| 2 | 140.36 | | N | | | |
| 3 | 174.68 | | N | | | |
| 4 | 126.10 | 5.71 | Y | H4→C1, | H4→δ_{P} | H1 |
| | (d; 10.60) | (d; 6.00) | | C2, C3, C5 | 18.47 ppm | |
| 5 | 177.02 | | N | | | |

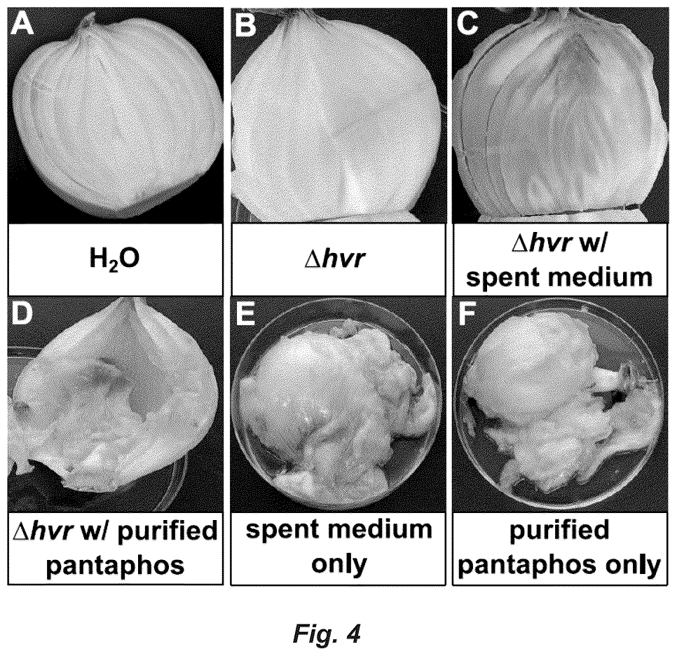

Lesions of onion center rot are caused by pantaphos. To investigate the role of hvr-associated phosphonates in pathogenesis, we repeated the onion rot assay using concentrated culture supernatants or purified pantaphos in the presence and absence the P. ananatis Ahvr mutant (FIG. 4). (Pure compound 2 was not obtained in sufficient quantities to allow bioactivity testing.) As described above, onions inoculated with P. ananatis Ahvr mutants showed minimal damage. However, when concentrated spent medium from IPTG-induced cultures of the phosphonate over-producing strain was co-inoculated with the Ahvr mutant, center rot was again observed. Similarly, co-inoculation with purified pantaphos also resulted in onion rot. Significantly, onions injected with either concentrated spent medium or purified pantaphos showed severe onion rot lesions in the absence of bacteria. The occurrence of center rot was dose-dependent with the characteristic lesions observed using as little as 90 μg (0.40 μmol) of pantaphos. Therefore, pantaphos is both necessary and sufficient to cause the center rot lesions in onions.

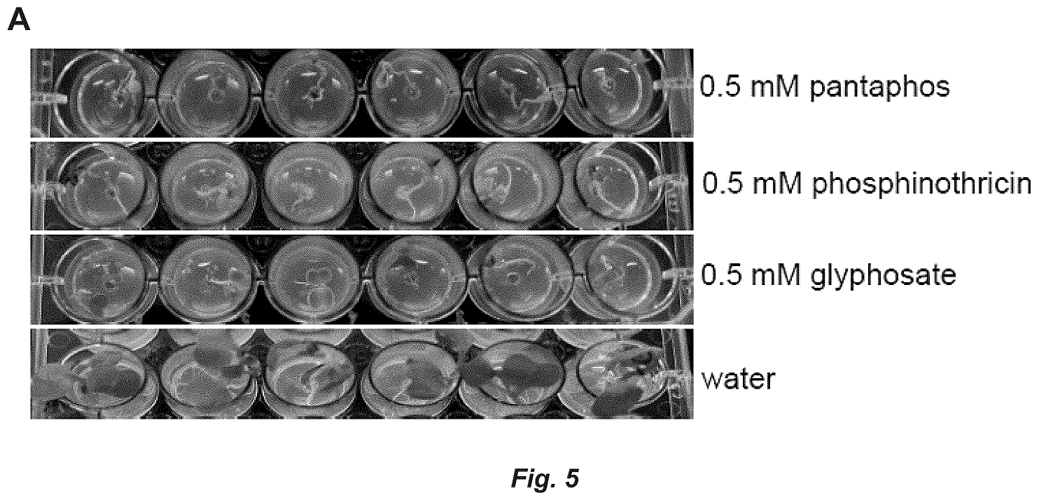

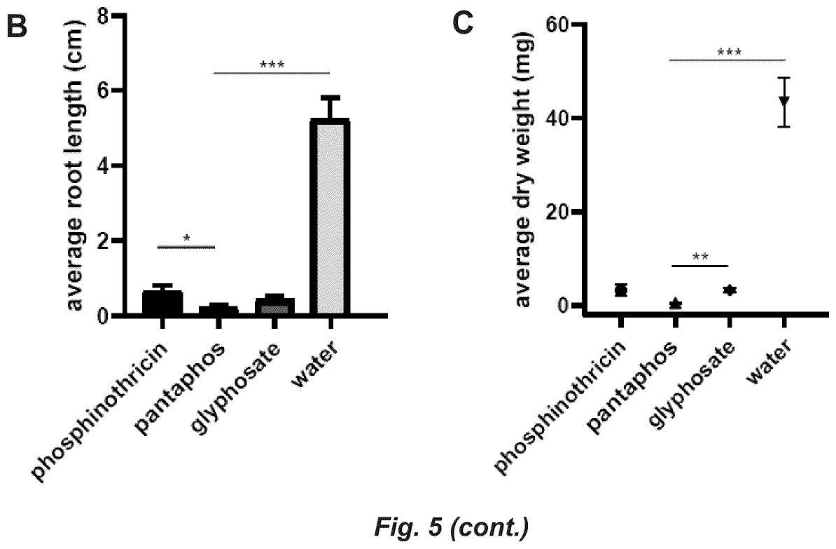

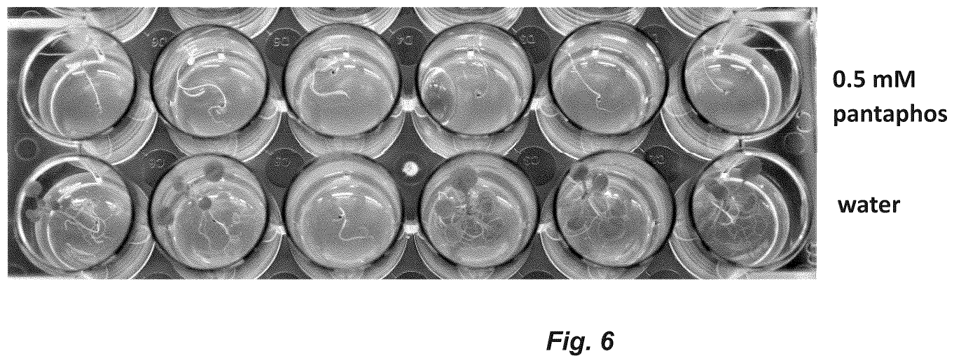

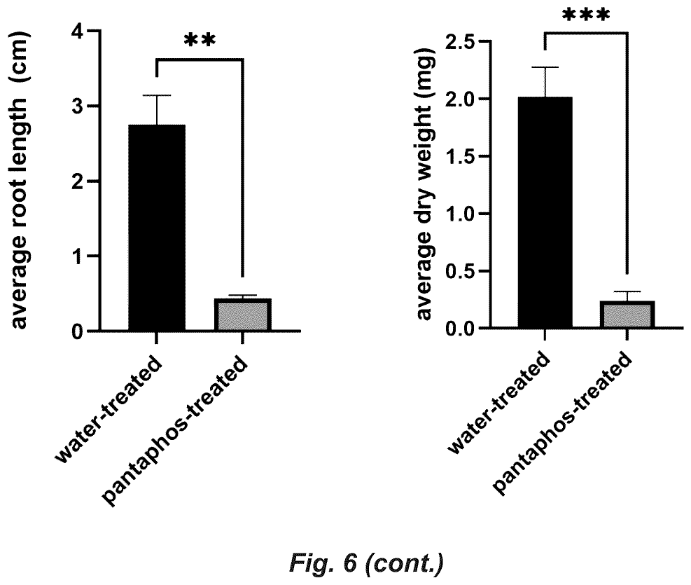

Phytotoxic effects of pantaphos treatment are comparable to known herbicides. To test whether the phytotoxicity observed in onion bulbs could be extended to growing plants, we treated newly germinated mustard seedlings (Brassica sp.) and Arabidopsis thaliana Col-0 seedlings with purified pantaphos using two well-characterized phosphonate herbicides (glyphosate and phosphinothricin) as controls (FIG. 5 and FIG. 6). After seven days growth, all three compounds caused a substantial reduction in root length and total dry weight of the seedlings relative to water-treated controls, with pantaphos being significantly more potent than phosphinothricin in the root length assay and than glyphosate in the dry weight assays. The phytotoxicity of pantaphos was dose-dependent with significant activity at concentrations 1.95 μM and above in the root length assay and above 31.3 μM in the dry weight assay.

Cytotoxic, antibacterial, and antifungal bioactivities of pantaphos. To examine whether bioactivity of pantaphos was specific to plants, we also conducted a series of bioassays against human cell lines, bacteria and fungi. Pantophos showed modest cytotoxicity to several human cell lines (Table 2). With the exception of one ovarian cancer cell line (ES-2), which was unaffected at the maximum dose, the IC₅₀ levels were roughly similar, in the range of 6.0 to 37.0 μM for each of the cell lines tested. One glioma cell line (A-172) was especially sensitive to pantaphos (IC₅₀ of 1.0 μM). In contrast, the molecule had no effect on the growth of fungi in rich or minimal media, including Candida albicans, Aspergillus fumigatus and two strains of Saccharomyces cerevisiae (Table 2). Similarly, a variety of Gram-negative and Gram-positive bacteria, including all of the so-called ESKAPE pathogens, were insensitive to pantaphos in both minimal and rich media (Table 2). To examine whether the insensitivity of E. coli was due to a lack of transport, we also tested bioactivity using the phosphonate-specific bioassay strain E. coli WM6242, which carries two copies of an IPTG-inducible, broad substrate-specificity phosphonate transporter. This strain was insensitive to pantaphos, with or without IPTG induction, suggesting that the lack of bioactivity in E. coli is not due to poor transport of the molecule.

TABLE 2
Bioactivity of pantaphos against human cells and microorganisms.

| Human cell line | IC₅₀ (μM)^{a} |
|---|---|
| HOS (human osteosarcoma) | 36.98 ± 6.28; Eₘₐₓ 58% |
| ES-2 (human ovarian cancer) | >100 |
| HCT-116 (human colon cancer) | 10.42 ± 2.00; Eₘₐₓ 59% |
| A-549 (human lung carcinoma) | 14.73 ± 0.61; Eₘₐₓ 66% |
| HFF-1 (human fibroblast cells) | 6.69 ± 0.29; Eₘₐₓ 85% |
| A-172 (human glioma cancer) | 1.01 ± 0.06; Eₘₐₓ 99% |

MIC (μM) for: ^{b}

| | Rich | Minimal |
|---|---|---|
| Fungi | medium | medium |
| Candida albicans SN250 | >125 | n.t.^{c} |
| Aspergillus fumigatus 1163 | >125 | >125 |
| Saccharomyces cerevisiae X2180-1A | >125 | >62.5 |

MIC (μM) for:^{d}

| | Rich | Minimal |
|---|---|---|
| Bacteria | medium | medium |
| Enterococcus faecalis ATCC 19433 | >200 | n.t.^{c} |
| Staphylococcus aureus ATCC 29213 | >200 | >200 |
| Klebsiella pneumoniae ATCC 27736 | >200 | >200 |
| Acinetobacter baumannii ATCC 19606 | >200 | >200 |
| Pseudomonas aeruginosa PAO1 | >200 | >200 |
| Escherichia coli ATCC 25922 | >200 | >200 |
| Salmonella enterica LT2 | >200 | >200 |
| Escherichia coli WM6242 −IPTG | >200 | >200 |
| Escherichia coli WM6242 +IPTG | >200 | >200 |

^{a}50% inhibitory concentration determined using the Alamar Blue method. Emax = percentage cell death.
^{b} Minimum inhibitory concentration (MIC) determined based on CLSI guidelines after 48 hrs growth. Bioactivity in rich medium was determined using RPMI 1640 medium. Bioactivity in minimal medium was determined using M9 minimal medium.
^{c}Not tested because the organism does not grow in minimal medium.
^{d}Minimum inhibitory concentration (MIC) determined based on CLSI guidelines. Bioactivity in rich medium was determined using Mueller-Hinton 2 medium and bioactivity in minimal medium was determined using glucose-MOPS minimal medium.

Putative functions of the hvr-encoded proteins and proposed pantaphos biosynthetic pathway. Combining the structures determined above with the proposed functions of the Hvr proteins suggests reasonable biosynthetic route for the P. ananatis phosphonates (FIG. 7). As with most phosphonate natural products, the pathway begins with the rearrangement of phosphoenolpyruvate (PEP) to phosphonopyruvate (PnPy) catalyzed by the enzyme PEP mutase. In P. ananatis, this reaction would be catalyzed by the HvrA protein, which is highly homologous to known PEP mutases. Because the PEP mutase reaction is highly endergonic (ΔG˜125 kJ/mol), subsequent steps must be highly favorable to drive net phosphonate synthesis. In the proposed pathway, this thermodynamic driving force is provided by the exergonic condensation of acetyl-CoA and phosphonopyruvate (PnPy) catalyzed by the HvrC protein, which is a homolog of the biochemically characterized phosphonomethylmalate (PMM) synthase involved in FR-900098 biosynthesis. PMM would then be dehydrated to form 2-phosphonomethylmaleate by HvrD and HvrE. These proteins are homologs of the small and large subunits of isopropylmalate dehydratase, respectively, which catalyze the isomerization of 3-isopropylmalate to 2-isopropylmalate via a dehydrated intermediate (2-isopropylmaleate) during leucine biosynthesis.

We expect that HvrDE will not catalyze the full reaction, but rather stop at the dehydrated intermediate. Precedent for this partial reaction is found in the propionate catabolic pathway of some bacteria, which catalyze the dehydration of 2-methylcitrate to 2-methyl-cis-aconitate using a member of the isopropylmalate dehydratase family. Conversion of 2-phosphonomethylmaleate to pantaphos is likely to be catalyzed by HvrB, a homolog of the flavin-dependent monooxygenases, NtaA and ScmK (48% and 47% identity, respectively). Consistent with the idea that this is an oxygen dependent reaction, we have observed that poorly aerated cultures accumulate 2-phosphonomethylmaleate instead of pantaphos. Flavin-dependent monooxygenases often require a separate flavin reductase to provide the electrons needed for reduction of oxygen to water. We propose that this function is provided by HvrK, a member of the flavin reductase family that is 30% identical to NtaB, which serves this function in the analogous NtaA-catalyzed reaction. Finally, we suggest that the HvrI protein, which is a member of the major facilitator superfamily, is responsible for export of the phosphonate products.

The proposed pathway for pantaphos biosynthesis uses only seven of the eleven genes in the hvr operon (Scheme 1). Based on homology to proteins of known function, three of remaining genes are predicted to encode an O-methyltransferase (HvrF), an N-acetyltransferase (HvrG) and an ATP-Grasp family protein (HvrH). The final unassigned protein (HvrJ) has no characterized homologs and, thus, we cannot predict a function. A twelfth protein that may, or may not, be part of the hvr operon also encodes an ATP-Grasp family protein (HvrL). Members of the ATP-Grasp family of enzymes often catalyze peptide bond formation. Accordingly, we suspect that peptidic derivatives of pantaphos may be produced by P. ananatis. Considering the absence of nitrogen in pantaphos, a peptidic derivative could also help explain the presence of the putative N-acetyltransferase HvrG, which might act as a self-resistance gene similar to the pat gene that confers self-resistance during biosynthesis of phosphinothricin tripeptide. Finally, the putative O-methyltransferase HvrF is highly homologous to trans-acontitate methyltransferase, which is thought to be involved in resistance to the spontaneously formed trans isomer of this TCA cycle intermediate. An analogous role can be envisioned for HvrF, if similar trans isomer side products are produced during pantaphos biosynthesis.

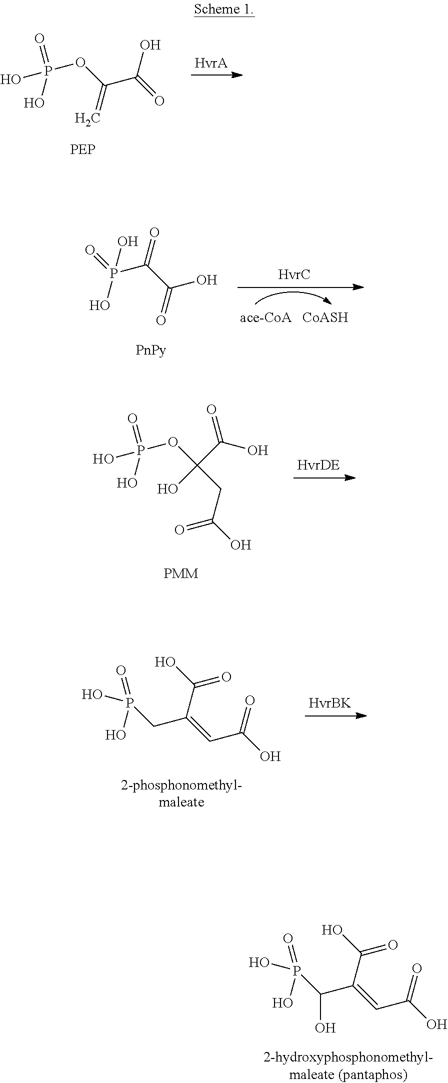

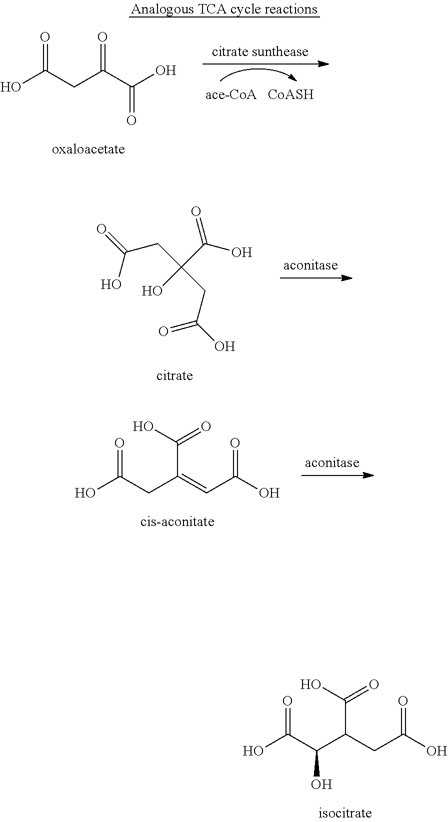 [SMILES: O=C(O)CC(=O)C(=O)O ; "CoASH" ; O=C(O)CC(O)(CC(=O)O)C(=O)O ; O=C(O)/C=C(/CC(=O)O)C(=O)O ; O=C(O)CC(C(=O)O)[C@@H](O)C(=O)O]

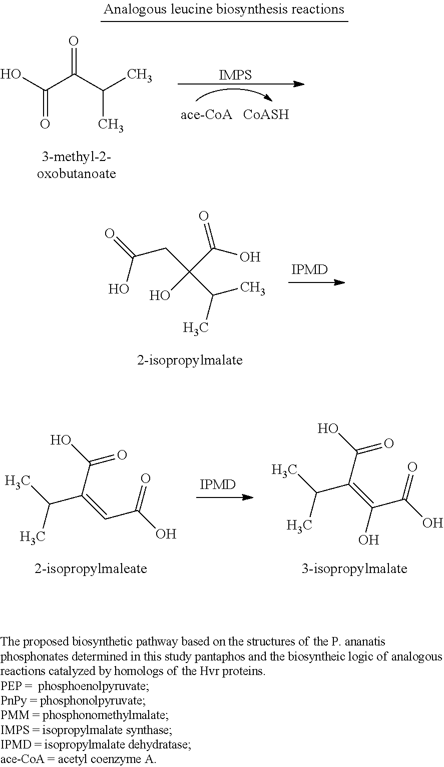

Homologs of the hvr operon in other bacteria. Asselin et al noted the presence of gene clusters similar to the hvr operon in a number of bacterial genome sequences. We felt it was worth revisiting this analysis in the light of the structures identified above. A total of thirty-three related gene clusters were identified in the NCBI genomic sequence databases using a combination of bioinformatics approaches. Based on the arrangement and the presence or absence of orthologous genes, nine types of Hvr-like biosynthetic gene clusters were observed. Putative orthologs of HvrA-F are conserved in all these groups, with the exception of Type VIII, which replaces the HvrDE proteins with a putative aconitase. As shown in FIG. 6, aconitase catalyzes a reaction that is essentially identical to the putative HvrDE reaction. Thus, we predict that the biosynthetic pathway encoded by the Type VIII hvr-like gene cluster has identical intermediates produced by paralogous enzymes. A feature that differentiates the nine types of hvr-like clusters is the presence or absence of one or more ATP-Grasp proteins, suggesting that a variety of peptidic derivatives of pantaphos might be produced in Nature. However, Type IX, which lacks ATP-grasp family proteins, likely has very different structural modifications compared to the other types, based on the presence of homologs to several additional enzyme families. Finally, the clusters also differ with respect to their putative export proteins and the presence/absence of a putative flavin reductase. The latter is not unexpected, as many flavin dependent enzymes can utilize generic reductases encoded by unlinked genes. Interestingly, the hvr-like clusters identified in our search were only found in a few of lineages within the proteobacteria and actinobacteria.

### Discussion

The phosphonate natural products produced by P. ananatis LMG 5342 are the principal virulence factor involved in onion center rot. Indeed, our data show that application of purified pantaphos produces identical lesions in the absence of bacteria. Although bioactive phosphonate natural products are well known, data supporting a direct role for these molecules in pathogenesis are rare. To date the sole known example is a complex phosphonate-modified polysaccharide produced by Bacteroides fragilis, which was shown to promote abscess formation in the mammalian gut. The demonstration that pantaphos is both necessary and sufficient for onion center rot adds a second phosphonate natural product to this short list and confirms the predicted function of the P. ananatis hvr locus proposed by Asselin et al (Mol Plant Microbe Interact 2018, 31:1291).

Despite the fact that unmodified pantaphos is phytotoxic, it seems likely that modified derivatives of the compound are also produced by P. ananatis. As described above, the hvr locus encodes two ATP-Grasp proteins. Members of this protein family often catalyze the ATP-dependent formation of peptide bonds, including those found in the peptidic phosphonate natural products rhizocticin, plumbemycin and phosphonoalamide. Addition of amino acid substituents often enhances uptake of bioactive compounds. For example, phosphinothricin tripeptide is a potent antibacterial compound, while unmodified phosphinothricin has poor activity. It should be noted, however, that these transport-mediated effects are species specific. Thus, phosphinothricin and phosphinothricin tripeptide are equally effective herbicides. A particularly striking example of specificity conferred by amino acid substituents is seen in the bioactivity profile of rhizocticin and plumbemycin. These natural products have the same bioactive phosphonate warhead attached to different amino acids, which presumably governs their uptake by specific peptide transport systems. As a result, rhizocticin is a potent antifungal agent that lacks antibacterial activity; whereas plumbemycin is a potent antibacterial that lacks antifungal activity. Based on these precedents, we suspect that peptidic derivatives of pantaphos may be produced during plant infection that increase its potency or specificity for a particular target species, perhaps accounting for the unstable compound 3 we observed in spent media.

Interestingly, pantaphos is not the only phosphonate produced by P. ananatis species. At least four distinct phosphonate biosynthetic gene clusters can be found in the currently sequenced P. ananatis genomes. With the exception of the hvr operon, neither the structures, nor the biological functions of the molecules produced by these gene clusters can be suggested at this time; however, our data clearly show that the pgb cluster is not required for onion center rot. Significantly, the strict correlation between plant pathogenicity and the presence of the hvr operon breaks down when larger numbers of Pantoea genomes are analyzed. Accordingly, P. ananatis strains PA4, PNA 14-1 and PNA 200-3 lack the hvr operon, but still cause onion disease; whereas strains PANS 04-2, PNA 07-10 and PNA 200-7 carry the hvr locus, but do not cause the disease. These data indicate that additional virulence traits are important for onion pathogenesis in Pantoea species. They also demonstrate the need for caution when using the presence/absence of the hvr cluster as a marker for plant pathogenicity.

Gene clusters similar to the hvr locus are relatively common in sequenced bacterial genomes; however, their phylogenetic distribution is rather restricted. Interestingly, many of the bacteria encoding these hvr-like gene clusters are associated with either plant or insect hosts. Homologs of the hvr operon are particularly abundant in members of the closely related Photorhabdus and Xenorhabdus genera. These bacteria have a unique lifestyle that relies on infection of an insect host via a nematode vector. Similarly, an hvr-locus is found in Bartonella senegalensis OSO₂, a putative intracellular pathogen of mammals. Given the cytotoxic effects we observed in human cells, it is tempting to suggest that pantaphos-like compounds may be involved in bacterial pathogenesis in both insects and humans. Homologs of the hvr operon are also seen in several members of the actinobacteria, including species of Streptomyces. These bacteria, which are common epiphytes and endophytes, are known for their production of a wide array of secondary metabolites including antibacterial, antifungal and phytotoxic compound. Thus, it seems likely that bioactive pantaphos-like molecules are important for mutualistic interactions of actinobacteria as well.

The idea that pantaphos and its putative derivatives are involved in diverse mutualistic interactions begs the question of the biological target for these molecules. Amongst the organisms tested to date, only plants and human cells show significant sensitivity to pantaphos, while bacteria and fungi were completely insensitive to the molecule. A simple explanation for these observations is that the target is shared by plants and animals and absent from bacteria and fungi. Alternatively, the target could be shared by all organisms, but be sufficiently different in bacteria and some fungi that it is not sensitive to the molecule. It is also possible that bacteria and some fungi share the ability to inactivate pantaphos, or that they are incapable of transporting the molecule into their cells. Significant experimental effort, beyond the scope of this initial report, will be required to distinguish between these possibilities; however, at least for E. coli, the data suggests that lack of transport is not responsible for the absence of bioactivity. Possible targets for pantaphos can be envisioned based on the structure of pantaphos, which is similar to a number of common metabolites including citrate, isocitrate, aconitate, isopropylmalate and maleate. Because most characterized phosphonates act as molecular mimics of normal cellular metabolites, this suggests the possibility that pantaphos may target the TCA cycle or leucine biosynthesis. Because maleic acid compounds are known to inhibit transaminases, it is also possible that the molecule inhibits the synthesis of another essential amine-bearing metabolite.

Finally, the studies described here have significant agricultural implications, which provide strong motivation for future studies on the biosynthesis and molecular target of pantaphos, as well as potential mechanisms for resistance to the compound. The identification of pantaphos as the principal virulence factor in onion rot suggests multiple approaches to deal with agricultural infestations of Pantoea ananatis. Because the molecule is required for virulence, it seems likely that inhibitors of the pantaphos biosynthetic pathway would prevent plant infection. It should also be possible to identify the molecular target of the compound in plants using purified or synthetic pantaphos, which would pave the way for development of crops with resistant alleles that would be immune to the disease. Plants expressing the putative pantaphos-modifying enzymes encoded by the hvr locus, or unrelated phosphonate catabolic genes, would also be expected to be specifically resistant to Pantoea infection. Finally, there is a desperate need to develop new treatments that are effective in combatting the alarming rise of herbicide-resistant weeds. The potent phytotoxicity of pantaphos suggests that it may have agricultural utility similar to that of the widely used phosphonate herbicides glyphosate and phosphinothricin. The real possibility of developing pantaphos-resistant crops strengthens this idea, as does the absence of bioactivity towards bacteria and fungi, suggesting that the molecule would have minimal effects on the soil microbiome. A note of caution is appropriate, however, given the moderate cytotoxicity we observed in human cell lines.

### General Synthetic Methods.

The invention also relates to methods of making the compounds and compositions of the invention. The compounds and compositions can be prepared by any of the applicable techniques of organic synthesis, for example, the techniques described herein. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as standard organic reference texts such as March's Advanced Organic Chemistry: Reactions, Mechanisms, and Structure, 5^{th} Ed. by M. B. Smith and J. March (John Wiley & Sons, New York, 2001), Comprehensive Organic Synthesis; Selectivity, Strategy & Efficiency in Modern Organic Chemistry, in 9 Volumes, Barry M. Trost, Ed.-in-Chief (Pergamon Press, New York, 1993 printing)); Advanced Organic Chemistry, Part B: Reactions and Synthesis, Second Edition, Cary and Sundberg (1983); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999).

A number of exemplary methods for the preparation of the compounds of the invention are provided below. These methods are intended to illustrate the nature of such preparations are not intended to limit the scope of applicable methods.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically, the temperatures will be −100° C. to 200° C., solvents will be aprotic or protic depending on the conditions required, and reaction times will be 1 minute to 10 days. Work-up typically consists of quenching any unreacted reagents followed by partition between a water/organic layer system (extraction) and separation of the layer containing the product.

Oxidation and reduction reactions are typically carried out at temperatures near room temperature (about 20° C.), although for metal hydride reductions frequently the temperature is reduced to 0° C. to −100° C. Heating can also be used when appropriate. Solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions). Standard synthetic techniques such as azeotropic removal of reaction by-products and use of anhydrous reaction conditions (e.g. inert gas environments) are common in the art and will be applied when applicable.

Protecting Groups. The term “protecting group” refers to any group which, when bound to a hydroxy or other heteroatom prevents undesired reactions from occurring at this group and which can be removed by conventional chemical or enzymatic steps to reestablish the hydroxyl group. The particular removable protecting group employed is not always critical and preferred removable hydroxyl blocking groups include conventional substituents such as, for example, allyl, benzyl, acetyl, chloroacetyl, thiobenzyl, benzylidene, phenacyl, methyl methoxy, silyl ethers (e.g., trimethylsilyl (TMS), t-butyl-diphenylsilyl (TBDPS), or t-butyldimethylsilyl (TBS)) and any other group that can be introduced chemically onto a hydroxyl functionality and later selectively removed either by chemical or enzymatic methods in mild conditions compatible with the nature of the product.

Suitable hydroxyl protecting groups are known to those skilled in the art and disclosed in more detail in T. W. Greene, Protecting Groups In Organic Synthesis; Wiley: New York, 1981 (“Greene”) and the references cited therein, and Kocienski, Philip J.; Protecting Groups (Georg Thieme Verlag Stuttgart, New York, 1994), both of which are incorporated herein by reference.

Protecting groups are available, commonly known and used, and are optionally used to prevent side reactions with the protected group during synthetic procedures, i.e. routes or methods to prepare the compounds by the methods of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis.

### Herbicidal Formulations.

In general, agrochemical formulations especially liquid form comprises either inorganic or organic solvents. Most of the known organic solvents known in the art are non-biodegradable and highly flammable. Organic solvents-based agrochemical formulations generally use a solvent that is preferably water-immiscible to dissolve the active component completely and produces a clear homogenous liquid free from extraneous matter. Alternatively, organic solvents typically have a low flash point, are non-biodegradable, cause skin irritation and possess medium or high evaporation rate etc. but provide a clear homogenous liquid. The known agrochemical compositions further include at least a surfactant wherein the performance and dosage of the included surfactant is based on the active content and solvent in the formulation, type of active ingredient, and solubility of the active ingredient in the solvent and the required emulsion performance of the final product.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, brain cancer, colon cancer, breast cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Methods and Materials

We constructed a P. ananatis LMG 5342 recombinant strain able to produce high quantities (>3 mg per liter) of a natively produced phosphonic acid small molecule produced from a biosynthetic gene cluster within a pathogenicity island (aka “HiVir” or hvr) of this bacterium. We have isolated and purified the small molecule and found it to be (2E)-3-carboxy-4-hydroxy-4-phosphonobut-2-enoate (compound 1) based on our nuclear magnetic resonance and mass spectrometry analyses. This small molecule acts in an herbicidal manner based on our data from herbicide bioassays with onion and mustard seedlings. The exact mechanism of action is unknown, but it produces similar toxicity as does the known herbicides phosphinothrcin and glyphosate. The DNA and protein sequences of P. ananatis may be found at ATGC database as disclosed in Kristensen et al., Nucleic Acids Res. 2017 Jan. 4; 45(D1):D210-D218. The ATGC database is hosted jointly at the University of Iowa at dmk-brain.ecn.uiowa.edu/ATGC/and the NCBI at ftp.ncbi.nlm.nih.gov/pub/kristensen/ATGC/atgc_home.html or at ftp.ncbi.nlm.nih.gov/pub/kristensen/ATGC/atgc_list.html. The database accession numbers for the nucleic acid sequences of the genes of the hvr operon of P. ananatis are hvrA (WP_013027161.1), hvrB (WP_041455823.1), hvrC (WP_013027159.1), hvrD (WP_014605075.1), hvrE (WP_013027157.1), hvrF (WP_013027156.1), hvrG (WP_014605077.1), hvrH (WP_013027154.1), hvrI (WP_013027153.1), hvrJ (WP_014605079.1) hvrK (WP_013027151.1), and hvrL (WP_013027150.1). Whole genome sequences of P. ananatis also may be found using Genbank accession number HE617160.1, NC_016816.1, HE617161.1, or NC_016817.1

Bacteria, plasmids and growth conditions. Bacterial strains, plasmids and primers used in this study are described in Table 3 and 4. General chemical and molecular biology reagents were purchased from Sigma-Aldrich or New England Biolabs. Phosphinothricin (glufosinate) was purchased from GoldBio (CAS #77182-82-2). Standard bacterial growth media were prepared as described (Current Protocols in Molecular Biology 2019, 125:e81, e82, e83). E. coli strains were typically grown at 37° C.; P. ananatis strains were typically grown at 30° C. Phosphonate induction media (PIM) was prepared as follows: 8.37 g/L MOPS, 0.72 g/L Tricine, 0.58 g/L NaCl, 0.51 g/L NH₄Cl, 1.6 g/L KOH, 0.1 g/L MgCl₂·6H₂O, 0.05 g/L K₂SO₄, 0.2 g/L K₂HIPO₄ were dissolved H₂O and steam sterilized for 20 min at 121° C. After cooling, 1× sterile trace element solution was added (1000× stock: 1.5 g/L nitrilotriacetic acid trisodium salt, 0.8 g/L Fe(NH₄)₂(SO₄)₂·7H₂O, 0.2 g/L Na₂SeO₃, 0.1 g/L CoCl₂·6H₂O, 0.1 g/L MnSO₄·H₂O, 0.1 g/L Na₂MoO₄·2H₂O, 0.1 g/L Na₂WO₄·2H₂O, 0.1 g/L ZnSO₄·7H₂O, 0.1 g/L NiCl₂·6H₂O, 0.01 g/L H₃BO₃, 0.01 g/L CuSO₄·5H₂O) followed by addition of 1% (v/v) sterile glycerol and 50 μg/mL sterile kanamycin. For culturing of Candida and Saccharomyces strains, Rosewell Park Memorial Institute 1640 medium (RPMI; Sigma-Aldrich R6504) was used for liquid culturing and Sabouraud Dextrose Agar (SDA; Difco™ 210950) for solid plating unless otherwise specified. For Aspergillus strains, Rosewell Park Memorial Institute 1640 medium (RPMI) was used for liquid culturing and Potato Dextrose Agar (PDA; Difco™ 213400) was used for solid plating unless otherwise specified. YMM medium (6.7 g/L yeast nitrogen base without amino acids [BD Difco™ 291940], 20 g/L glucose, 1× trace element solution) was used as minimal media for fungal strains.

TABLE 3
Microorganisms used in this study.

| | | Source/ |
|---|---|---|
| Strain | Genotype/Construction | Reference |
| Escherichia coli | | |
| DH5α/λpir | F- endA1 glnV44 thi-1 recA1 relA1 gyrA96 deoR nupG | a, b |
| | Φ80dlacZ ΔM15 Δ(lacZYA-argF)U169, hsdR17(rK- | |
| | mK⁺), λpir | |
| WM6026 | lacI^{q}, rrnB3, ΔlacZ4787, hsdR514, ΔaraBAD567, | c |
| | ΔrhaBAD568, rph-1, attλ::pAE12(ΔoriR6K- | |
| | cat::Frt5), ΔendA::Frt, uidA(ΔMluI)::pir, attHK::pJK100 | |
| | 6 Δ(oriR6K-cat::Frt5; trfA::Frt) | |
| WM6242 | lacI^{q}, rrnB3, Δ(lacZ4787), hsdR514, attP22(EcoB), | d |
| | Δ(araBAD)567, Δ(rhaBAD)568, rph-1, Δ(phnC-P), | |
| | Δ(phoA), HKattB::pJK077(ΔaadA-oriR6K), lambda- | |
| | attB::pJK074(Δcat-oriR6K) | |

Pantoea ananatis

| | | |
|---|---|---|
| LMG 5342 | Native phosphonate producer | ATCC #22920 |
| B-133 | wild-type | ARS NRRL |
| B-14773 | wild-type | ARS NRRL |
| MMG1888 | Δpgb Δhvr; Markerless deletion in MMG1984 using | This study |
| | allele exchange plasmid pAP04 | |
| MMG1904 | Δphn; Markerless deletion in LMG 5342 using allele | This study |
| | exchange plasmid pAP05 | |
| MMG1912 | Δhvr; Markerless deletion in LMG 5342 using allele | This study |
| | exchange plasmid pAP04 | |
| MMG1920 | Δphn Δhvr; Markerless deletion in MMG1904 using | This study |
| | allele exchange plasmid pAP04 | |
| MMG1984 | Δpgb; Markerless deletion in LMG 5342 using allele | This study |
| | exchange plasmid pAP02 | |
| MMG1988 | Δpgb Δphn; Markerless deletion in MMG1904 using | This study |
| | allele exchange plasmid pAP02 | |
| MMG2010 | Δphn Δpgb hvr::pAP01; pAP01 recombinant host for | This study |
| | over-expression of Hvr-BGC | |
| MMG2012 | Δpgb Δphn Δhvr; Markerless deletion in MMG1988 | This study |
| | using allele exchange plasmid pAP04 | |

| | |
|---|---|
| Enterococcus faecalis ATCC 19433 | ATCC #19433 |
| Staphylococcus aureus ATCC 29213 | ATCC #29213 |
| Klebsiella pneumoniae ATCC 27736 | ATCC #27736 |
| Acinetobacter baumannii ATCC 19606 | ATCC #19606 |
| Pseudomonas aeruginosa PAO1 | Hergenrother |
| | Lab (UIUC) |
| Escherichia coli ATCC 25922 | ATCC #25922 |
| Salmonella enterica LT2 | ATCC #700720 |
| Candida albicans SN250 | Burke Lab (UIUC) |
| Aspergillus fumigatus 1163 | Burke Lab (UIUC) |
| Saccharomyces cerevisiae X2180-1A | Imlay Lab (UIUC) |

a Cell 1978, 15(4): 1199-208
b Proc Natl Acad Sci USA 1990, 87(12): 4645-4649
c Nat Chem Biol 2007, 3: 480-485
d Chem Biol 2008, 15(8): 765-770

Bioinformatic analyses of phosphonate metabolism in P. ananatis strains. P. ananatis genomes were downloaded from NCBI database and Hidden Markov Model (HMM) search was performed using HMMER version 3.2.1. The HMMs used for analysis were phosphoenolpyruvate phosphomutase (pepM, TIGR02320), phosphonoacetaldehyde hydrolase (phnX, TIGR01422), phosphonoacetate hydrolase (phnA, TIGR02335), 2-AEP transaminase (phnW, TIGR02326 and TIGR03301), phosphonopyruvate hydrolase (palA, TIGR02321), phosphonoacetaldehyde dehydrogenase (phnY, TIGR03250), HD-phosphohydrolase (phnZ, TIGR00277 and PF01966), C—P lyase (phnJ, PF06007). Genomes with an HMM match were considered to have the associated metabolism. Genomes containing pepM were screened for the presence of the hvr locus by mapping them to the P. ananatis LMG 5342 gene cluster using Geneious Prime® 2020.2.1 software. For pepM genes that did not correspond to the hvr operon, gene cluster boundaries around the pepM were deduced based on the presence of either flanking integrative and conjugative elements or genes that appeared to be in an operon together with pepM. BLASTP was used to assign functions to genes in the gene cluster based on homology to proteins of known function.

Genome sequencing of Pantoea NRRL strains. The genomes of strains P. ananatis NRRL B-14773 and P. ananatis B-133 (since renamed as P. stewartii based on NCBI average nucleotide identity analyses) were sequenced for use in this study. High molecular weight genomic DNA was purified using Qiagen® DNeasy UltraClean Microbial Kit. Purified genomic DNA was prepared using Shotgun Flex DNA library prep and sequenced using Illumina MiSeq v2 platform (250 nt paired end reads) by the Roy J. Carver Biotechnology Sequencing Center, UIUC. Genome reads were trimmed using BBDuk software, assembled using SPAdes 3.14.1 and annotated using RAST Server. Assembled reads were submitted to the NCBI Whole Genome Shotgun (WGS) database. This WGS project has been deposited at DDBJ/ENA/GenBank under the accessions JACETZ000000000 (for P. stewertii NRRL B-133 and JACEUA000000000 (for P. ananatis NRRL B-14773). Genomes were analyzed for phosphonate metabolism as described above.

TABLE 4
Plasmids used in this study.

| | | Source/ |
|---|---|---|
| Plasmids | Features/Construction/Use | Reference |
| pAE4 | oriT, Apr^{R}, λattP, ΦC31 int, ΦC31attP | d |
| pAH56 | uidAF, λattP, oriR6K, Kan^{R}, lacI^{q}, Ptac | e |
| pAP01 | hvrA, oriT, λattP, oriR6K, Kan^{R}, lacI^{q}, Ptac; Gibson assembly | This study |
| | with pAP10 PCR product (PCR primers pAP10-rev/for) and | |
| | P. ananatis LMG 5342 pepM gene (PCR primers Hvr-PepM- | |
| | rev/for); used for integrating Ptac promotor upstream of Hvr-BGC | |
| pAP02 | sacB, Amp^{R}, oriR6K, T7ₚ, Kan^{R}, lacₚ; Gibson assembly with | This study |
| | pHC001A-SacI/XhoI-digested plasmid and 1 kb upstream (PCR | |
| | primers Pgb-left-hArm_R/F) and downstream (PCR primers | |
| | Pgb-right-hArm_R/F) homology fragments to Pgb gene cluster; | |
| | used to make markerless deletion of Pgb gene cluster | |
| pAP04 | sacB, Amp^{R}, oriR6K, T7ₚ, Kan^{R}, lacₚ; Gibson assembly with | This study |
| | pHC001A-SacI/XhoI-digested plasmid and 1 kb upstream (PCR | |
| | primers Hvr-left-hArm_R/F) and downstream (PCR primers | |
| | Hvr-right-hArm_R/F) homology fragments to Hvr gene cluster; | |
| | used to make markerless deletion of Hvr gene cluster | |
| pAP05 | sacB, Amp^{R}, oriR6K, T7ₚ, Kan^{R}, lacₚ; Gibson assembly with | This study |
| | pHC001A-SacI/XhoI-digested plasmid and 1 kb upstream (PCR | |
| | primers Phn-left-hArm_R/F) and downstream (PCR primers | |
| | Phn-right-hArm_R/F) homology fragments to Phn gene cluster; | |
| | used to make markerless deletion of Phn gene cluster | |
| pAP10 | uidAF, oriT, λattP, oriR6K, Kan^{R}, lacI^{q}, Ptac; Gibson assembly | This study |
| | with pAH56-SalI-digested plasmid and the oriT PCR-fragment | |
| | from pAE4 plasmid (PCR primers pAH56-pAE4oriT-rev/for); | |
| | used as parent plasmid for construction of pAP01 | |
| pHC001A | sacB, Amp^{R}, oriR6K, T7ₚ, Kan^{R}, lacₚ; used as parent plasmid | f |
| | for allele exchange plasmid construction | |

d Chem Biol 2008, 15(8): 765-70
e J Bacteriol 2001, 183(21): 6384-93
f J Biol Chem 2011, 286(25): 22283-90

Genetic methods for making unmarked deletion mutations. Approximately 1 kb of DNA upstream and downstream of the region to be deleted were cloned into pHC001A (see Table 4 for complete list of plasmid constructs). The resulting plasmids were introduced into electrocompetent E. coli DH5α/λpir and maintained in LB media+50 μg/mL kanamycin (LB-Kan). Plasmid constructs were moved into the conjugation strain WM6026 via electroporation-mediated transformation, then transferred to P. ananatis recipients via conjugation. Conjugations were performed by streaking isolated colonies of donor and recipient together in small (2 cm×2 cm) patches on agar-solidified LB medium containing 60 μM diaminopimelic acid (DAP) to allow growth of WM6026-derived donor strains, which are DAP auxotrophs. After overnight incubation at 30° C., the patches were picked and restreaked on LB-Kan without added DAP to select for P. ananatis recombinants that carry the deletion plasmids, which cannot replicate autonomously in P. ananatis, inserted into the target locus by homologous recombination. Exconjugants were purified by streaking for isolated colonies on LB-Kan at 30° C., then by streaking on LB without antibiotics to allow for segregation of the integrated plasmid. Recombinants that had lost the integrated plasmid were then isolated by streaking on LB media without NaCl containing 5% sucrose, which selects against the sacB gene encoded on the integrated deletion plasmid. Loss of the integrated plasmid was verified by showing that the purified recombinants were kanamycin sensitive. Finally, recombinants carrying the desired deletion were identified by PCR-based screening using primers (Table 5) described in Polidore et al., mBio. 2021 January-February; 12(1): e03402-20, which reference and supporting information is incorporated herein by reference in its entirety.

TABLE 5
Primers used in this study.

| Primer Name | Sequence (5′→3′) | Use |
|---|---|---|
| Pgb-int-rev | CTTGCTGCAGGTAGGGGT | PCR-based assay for detection of |
| | (SEQ ID NO: 26) | phosphocholine cytidylyltransferase |
| Pgb-int-for | TCTATCCACGGCAAACCACT | gene (n.t. 2,701,405->2,700,635)^{a} |
| | (SEQ ID NO: 27) | within Pgb gene cluster |
| dPgb-rev | TGATGGCCTGCAAGACGG | PCR-based assay for detection of |
| | (SEQ ID NO: 28) | the deletion of Pgb gene cluster |
| dPgb-for | TCTATCCACGGCAAACCACT | |
| | (SEQ ID NO: 29) | |
| Phn-int-rev | CAGCGCAACAGACTGGGA | PCR-based assay for detection of |
| | (SEQ ID NO: 30) | alpha-D-ribose 1- |
| | | methylphosphonate 5-triphosphate |
| Phn-int-for | CCCATTCCGCCATGAGCA | diphosphatase gene (phnM; n.t. |
| | (SEQ ID NO: 31) | 1,796,249->1,797,385)ª within |
| | | Phn gene cluster |
| dPhn-rev | ACGGTAAGATTGGGCGCC | PCR-based assay for detection of |
| | (SEQ ID NO: 32) | the deletion of Phn gene cluster |
| dPhn-for | GGCCAACGATCGCGGATA | |
| | (SEQ ID NO: 33) | |
| PANA_3283 | GCTGCTATCCCCGAGATAATGA | PCR-based assay for detection of |
| 812-833R | (SEQ ID NO: 34) | MFS transporter gene (hvrI; |
| PANA_3283 64- | GCTGAAGGGATTCAGACGGTTA | 809,348->810,584)ª within Hvr |
| 85F | (SEQ ID NO: 35) | gene cluster; primer sequences |
| | | taken from the reference below^{g} |
| dHvr-rev | TTACCGCCACCTTGCTGG | PCR-based assay for detection of |
| | (SEQ ID NO: 36) | the deletion of Hvr gene cluster |
| dHvr-for | TTTCGCCCGTTCCCCTTC | |
| | (SEQ ID NO: 37) | |
| PANA-Hvr- | GCCGTCCTGCCATATCTCAA | Detection of Hvr marker gene; |
| pepM-rev | (SEQ ID NO: 38) | Phosphoenolypyruvate mutase gene |
| PANA-Hvr- | TAACGGACTCAGCATCTCGC | (hvrA; n.t. 801,910->802,764)ª |
| pepM-for | (SEQ ID NO: 39) | |
| pHC001A-MCS- | CCCGACCCGAAACACCAT | Sequencing primer |
| rev | (SEQ ID NO: 40) | |
| pHC001A-MCS- | TGTTCGCCAGGCTCAAGG | Sequencing primer |
| for | (SEQ ID NO: 41) | |
| Phn-left- | ACTAAAGGGAACAAAAGCTGGA | For pAP05 construction |
| hArm_R | GCTCCGCCAAAAATCAGCTGTG | |
| | (SEQ ID NO: 42) | |
| Phn-left- | GGATTTAATTGTGAAAGACTCTC | |
| hArm_F | CGCTCGTG | |
| | (SEQ ID NO: 43) | |
| Phn-right- | GCGGAGAGTCTTTCACAATTAA | |
| hArm_R | ATCCTCACATCAGTAGAGG | |
| | (SEQ ID NO: 44) | |
| Phn-right- | CTGGATGATCCTCCAGCGGGGC | |
| hArm_F | CCCCCCTCGAGTTCATGGCGCGG | |
| | CTTTCG | |
| | (SEQ ID NO: 45) | |
| Pgb-left- | ATTTTATTTATTTAAACGTTAAA | For pAP02 construction |
| hArm_R | CAAGAAATTCATC | |
| | (SEQ ID NO: 46) | |
| Pgb-left- | TCCTCCAGCGGGGCCCCCCCTCG | |
| hArm_F | AGTTGAAGCGGCTAACTTCC | |
| | (SEQ ID NO: 47) | |
| Pgb-right- | AGGGAACAAAAGCTGGAGCTCT | |
| hArm_R | TGTTTCATCCATCATACC | |
| | (SEQ ID NO: 48) | |
| Pgb-right- | TTAACGTTTAAATAAATAAAATT | |
| hArm_F | GCTTGTCTCATG | |
| | (SEQ ID NO: 49) | |
| Hvr-left- | CTGACGGATTTTACAAACGCAA | For pAP04 construction |
| hArm_R | AAACCCCCGCC | |
| | (SEQ ID NO: 50) | |
| Hvr-left- | CTGGATGATCCTCCAGCGGGGC | |
| hArm_F | CCCCCCTCGAGTAATCGCCGCCC | |
| | ACGCCG | |
| | (SEQ ID NO: 51) | |
| Hvr-right- | ACTAAAGGGAACAAAAGCTGGA | |
| hArm_R | GCTCCCATCATTACGTTTATGCC | |
| | (SEQ ID NO: 52) | |
| Hvr-right- | GTTTTTGCGTTTGTAAAATCCGT | |
| hArm_F | CAGGTGCAC | |
| | (SEQ ID NO: 53) | |
| SEQ-pAP-rev | ACTATGAGCACGTCGGCG | sequencing primer |
| | (SEQ ID NO: 54) | |
| SEQ-pAP-for | CGCACTCCCGTTCTGGAT | sequencing primer |
| | (SEQ ID NO: 55) | |
| aph-rev | CAGGATGAGGATCGTTTCGC | Kan^{R} marker gene detection |
| | (SEQ ID NO: 56) | |
| aph-for | TCGAACCCCAGAGTCCCG | Kan^{R} marker gene detection |
| | (SEQ ID NO: 57) | |
| pAP10-rev | ATGTATATCTCCTTCTTACAAGC | For pAP01 construction |
| | TTGGC | |
| | (SEQ ID NO: 58) | |
| pAP10-for | TCTAGATGCACTCCACCGCTGAT | |
| | GACATCAG | |
| | (SEQ ID NO: 59) | |
| Hvr-PepM-rev | ATGTCATCAGCGGTGGAGTGCA | |
| | TCTAGATTAAGGAATCAGTGAA | |
| | ATAATTTC | |
| | (SEQ ID NO: 60) | |
| Hvr-PepM-for | AAGCTTGTAAGAAGGAGATATA | |
| | CATATGATCAAAAAACTTATTGC | |
| | AG | |
| | (SEQ ID NO: 61) | |
| pAH56- | CATGAGAATTAATTCCGGGGAT | For pAP10 construction |
| pAE4oriT-rev | CCGTCGACACTACCATCGGGGG | |
| | CCATC | |
| | (SEQ ID NO: 62) | |
| pAH56- | CTACAGCCTCGGGAATTGCTGC | |
| pAE4oriT-for | AGGTCGACTCTAGATGCACTCC | |
| | ACCGC | |
| | (SEQ ID NO: 63) | |

^{g}Mol Plant Microbe Interact 2018, 31(12):1291-1300

Introduction of IPTG inducible Ptac system. Plasmid construct, pAP01, was introduced into WM6026 by electroporation with selection on LB+50 μg/mL kanamycin (LB-Kan). The plasmid was then transferred to P. ananatis MMG1988 as described in the preceding section. Recombinants that carry an integrated copy of pAP01 inserted into the hvrA gene were selected on LB-Kan. The resulting colonies were screened for the plasmid integration using primers for aph and lacI^{q} as well as the Hvr-marker gene hvrI. The resulting strain, P. ananatis MMG2010, was maintained in LB-Kan to prevent loss of the integrated plasmid.

NMR and MS. The ¹H-NMR, ¹³C-NMR and ³¹P-NMR spectra were recorded on an Agilent DD2 600 MHz spectrometer (600 MHz for ¹H, 150 MHz for ¹³C and 243 MHz for ³¹P). Samples were prepared in 20-100% D20 as the locking solvent. Quantitative ³¹P NMR was performed using an internal standard of 0.5 mM dimethylphosphinate with addition of 0.9 mM EDTA, and acquisition was performed using 5× the T1 measurement (relaxation time) for the sample. Phosphonate peak integrals were calculated using MestReNova v11.0.1 software and normalized to the internal standard. Concentrations were calculated based on the ratio of the normalized phosphonate peak integrals to the known concentration of internal standard. Mass spectrometry was performed by the School of Chemical Sciences Mass Spectrometry Laboratory using a Waters® Q-TOF Ultima ESI in which 10 μL of sample was injected at a concentration of 10 μg/mL in methanol.

IPTG-induced expression of the hvr operon in P. ananatis AIIG2010. A frozen glycerol stock of P. ananatis MMG2010 was revived on LB+50 μg/mL kanamycin and incubated at 30° C. for 24 hours. A single colony was then transferred to 5 mL of phosphonate induction media (PIM). The culture was incubated at 30° C. for 48 hours, then 0.5 mL of culture was transferred to 50 mL PIM and incubated at 30° C. for 24 hours. The next day, 8 mL of culture was transferred to each of four flasks containing 800 mL PIM medium plus 1 mM IPTG. These 800 mL cultures were incubated shaking at 175 rpm at 30° C. for 72 hours. After growth, cultures were centrifuged at 8000 rpm for 20 minutes to remove cells and debris and the supernatant was concentrated by freeze-drying. Quantitative ³¹P NMR analysis was performed on the concentrated supernatant aliquot to determine phosphonate production levels after adding dimethylphosphinate (0.5 mM final) as an internal standard.

Purification of pantaphos and compound 2. A 3.2 L culture of P. ananatis MMG2010 was grown in PIM medium with 1 mM IPTG as described in the preceding section. After centrifugation to remove cells, the spent medium was freeze-dried, and the dried material resuspended in 300 mL H₂O. 1200 mL of 100% cold methanol was then added for a final concentration of 75% methanol and incubated at −20° C. overnight. Precipitated material was removed and saved using vacuum filtration with a Whatman® grade 42 ashless filter and the methanol-soluble fraction dried to completion using initial rotary evaporation followed by freeze-drying. The 48.0 g of dried material obtained by this process (Sample A) was saved for further purification. The precipitated material saved from the above methanol extraction was subject to a secondary 75% methanol extraction as described. Then the methanol-soluble fraction was dried to completion using initial rotary evaporation followed by freeze-drying. The 1.60 g of dried material obtained by this process (Sample B) was saved for further purification. Sample A was subject to Fe³⁺-IMAC purification as follows. Ten grams of Chelex resin (sodium form) was converted to the H⁺ form by incubation in 1 M HCl for 30 minutes followed by wash with 5 column volumes (CV) of water. Next, the resin was charged with Fe⁺³ by resuspension in 100 mL of 300 mM FeCl₃·6H₂O for 1 hour at 4° C. followed by washing with 100 mL of 0.1% acetic acid and incubation in 100 mL of 0.1% acetic acid overnight at 4° C. Sample A was acidified using concentrated acetic acid to pH 3 and incubated with the Fe-IMAC resin at 4° C. for 2 hours. The solution was separated from the resin using a gravity column and the flow-through containing unbound phosphonic acids was saved (Sample-Flowthrough). Bound phosphonic acids were eluted from the Fe-IMAC resin using a gradient of 100 mL NH₄HCO₃ (1, 5, 25, 50, 100, 250, 500, and 1000 mM) and fractions collected and neutralized to neutral pH using acetic acid. The eight fractions were concentrated using initial rotary evaporation followed by freeze-drying. Sample-Flowthrough, containing any unbound phosphonic acids, obtained from above was combined with Sample B and subjected to a second round of Fe-IMAC purification as described above. Fractions were concentrated using initial rotary evaporation followed by freeze-drying. From each of the Fe-IMAC purifications, the fractions were combined in 2-3 mL H₂O as follows: the 500-1000 mM NH₄HCO₃ fractions (Sample 1), the 1-5 mM NH₄HCO₃ fractions (Sample-2), and the 25-50-100-250 mM NH₄HCO₃ fractions (Sample 3). Samples 1, 2, and 3 were separately concentrated and dried via freeze-drying to obtain 45.1 mg, 30.6 mg, and 6.9 mg of dried material respectively. Quantitative phosphorus NMR, based on a 0.5 mM dimethylphosphinate standard reference, and purity assessment using proton NMR was performed for each sample. Samples 1 and 3 contained 0.551 mmol and 0.215 mmol phosphonate respectively. However, Sample 2 was found to contain only 0.0165 mmol phosphonate and included residual phosphates that are not present in Samples 1 and 3, therefore, was not used for further purification steps.

Sample 1 was subjected to further purification using a Teledyne ISCO CombiFlash RF+ UV-Vis system using a RediSep SAX anion exchange resin. Sample 1 was lyophilized, then reconstituted in 75% methanol. Insoluble material was removed by centrifugation, resuspended in 1 mL of 100% D20 and examined by ³¹P NMR. Samples containing residual phosphonates were subjected to additional cycles of drying and 75% methanol until all phosphonate compounds were solubilized. The methanol-soluble fractions were then pooled and subjected to CombiFlash purification. To do this, 5.7g RediSep SAX column was equilibrated with 20 column volumes (CV) of 5% NH₄OH in H₂O, followed by 20 CV of H₂O, then 20 CV of 90% methanol. A 1 mL sample from the methanol-soluble fractions was loaded onto the column via direct injection followed by: 3.3 min 100% A (90% methanol), linear gradient to 100% B (5% NH₄OH in H₂O) over 15 min then 100% B for 6 min followed by 3.5 min 100% A at 18 mL/min flow rate. Fractions were monitored using UV 250 nm and 210 nm, fractions showing absorbance at either wavelength were combined and analyzed by ³¹P NMR. Fractions containing the δ_{P} 18 and 15 ppm phosphorus chemical shifts were saved and dried via rotary evaporation followed by freeze-drying.

Sample 3 and the Combiflash purified Sample 1 were then subject to HPLC purification separately. Dried samples were reconstituted in 1 mL H₂O, then 50-100 μL of sample was diluted in solvent B (90% acetonitrile+10 mM NH₄HCO₃ at pH 9.20) for a final concentration of 75% solvent B. Samples were then filtered through a 0.45 μm filter and purified using Atlantis HILIC Silica column (10×250 mm², 5 μm particle size) using gradient elution. Chromatography was performed at flow rate of 4 mL/min using H₂O+10 mM NH₄HCO₃ at pH 8.50 (solvent A) and 90% acetonitrile+10 mM NH₄CO₃ at pH 9.20 (solvent B). The gradient performed was as follows: 8 min at 90% solvent B, followed by a linear gradient to 70% solvent B over 20 min, then 50% solvent B over 1 min, hold at 50% solvent B over 8 min, then back to 90% solvent B over 1 min, followed by hold at 90% solvent B for 8 min. Fractions were collected and monitored for UV absorption at 210 and 250 nm. Fractions that absorbed at these wavelengths were combined and dried via rotary evaporation and analyzed using phosphorus NMR. Fractions were obtained that contained a pure phosphonate compound with a δ_{P} 15 ppm chemical shift (corresponding to pantaphos), and fractions were obtained containing a purified phosphonate compound with a r 18 ppm chemical shift (corresponding to compound 2). Additional fractions were obtained that contained a mixture of pantaphos and compound 2. Purified compounds were dried and saved at 4° C. for MS and NMR structural analyses as described above.

Onion bioactivity assays. Yellow onions purchased from a local market were surface sterilized in a laminar flow biosafety hood as follows. First, the outermost layers with any damage or browning were removed and discarded, followed by soaking for 10 minutes in 10% bleach. The onions were then rinsed three times with sterile dH₂O, followed by soaking in 70% EtOH for an additional 10 minutes, then rinsed four times with sterile dH₂O. The onions were left in a biosafety laminar flow hood until water and ethanol were completely evaporated. For testing virulence of microbial strains, the onions were inoculated by stabbing with a previously sterilized wooden toothpick dipped into a bacterial cell suspension (1×10³ CFU/mL in 1×PBS buffer). Chemical complementation studies were performed by addition of 100 μL of filter-sterilized sample into the hole created by the toothpick during inoculation. For testing bioactivity of crude and purified chemical samples in the absence of bacteria, holes were punched in sterilized onions using sterile toothpicks followed by addition of 100 μL of a filter-sterilized sample. Following inoculation and/or treatment with filter-sterilized compounds, the onions were placed in zip-lock plastic bags and incubated at 30° C. in the dark for the indicated number of days. Following incubation, the onions were sectioned across the site of inoculation/sample application to allow visual inspection of the center rot phenotype.

Mustard seedling and Arabidopsis thaliana Col-0 bioactivity testing. All seed preparation was performed in a laminar flow biosafety hood. Burpee® tendergreen mustard seeds were cleaned as follows: 10% bleach for 1 min, 3× rinse with sterile dH₂O, 70% ethanol for 1 min, followed by 5× rinse with sterile dH₂O. Washed seeds were transferred to a sterilized paper towel and placed inside a sterile container. Then, 5 mL of sterile tap water was added to the paper towel and the container was incubated in the dark for 48 hours to allow the seeds to germinate. Germinated seedlings were transferred to 24-well cell culture plates containing 1 mL of Murashige and Skoog agar (1% agar) per well. Arabidopsis thaliana Col-0 sterile seeds (Stock Number: CS1092) were purchased from the Arabidopsis Biological Resource Center (ABRC) and incubated at 4° C. for 2-3 days. Cold incubated seeds were then transferred to 24-well cell culture plates containing 1 mL of Murashige and Skoog agar (1% agar) per well and incubated in the dark until seed germination observed. To both mustard and Arabidopsis germinated seedlings, 20 μl of an appropriate dilution of the filter-sterilized compounds being tested were then added to each well to achieve the desired concentration. For negative controls, 20 μL of sterile dH₂O was spotted onto the agar. The 24-well plates were incubated in a 60% humidity-controlled growth room with a 16-hour light cycle at 23° C. for one week. Following incubation, plants were extracted from the growth agar by gentle pulling, which resulted in essentially agar free plants. Root length was measured immediately; dry weight was determined after drying for 24 hours at 150° C. Statistical analysis was performed among each condition to determine significance using the standard Welch's t-test analysis from GraphPad Prism 8.4.1 software.

Cell culture cytotoxicity screening. The compounds were evaluated for their ability to kill cancer cell lines in culture, using HOS (osteosarcoma); ES-2 (ovarian cancer); HCT 116 (colon cancer); A549 (lung carcinoma); and A172 (glioma) cells. Human skin fibroblast cells (HFF-1) were also assessed. Cells were seeded (3000 cells well⁻¹ for ES-2, HCT 116, A549 and A172; 4000 cells well⁻¹ for HFF-1 and 2500 cells well⁻¹ for HOS) in a 96-well plate and allowed to attach overnight. Cells were treated with pantaphos in water. The concentrations of the tested compounds were 5 nM to 100 μM (1% water final; 100 μl well⁻¹). Raptinal (50 μM) was used as a dead control. On each plate, five technical replicates per compound were performed. 72 hours post-treatment, cell viability was assessed using the Alamar Blue method (http://www.bio-rad-antibodies.com/measuring-cytotoxicity-proliferation-spectrophotometry-fluorescence-alamarBlue.html). Alamar Blue solution (10 μl of 440 μM resazurin in sterile 1×PBS) was added to each well, and the plate was incubated for 3-4 hrs. Conversion of Alamar Blue was measured with a plate reader (SpectraMax M3; Molecular Devices) by fluorescence (excitation wavelength: 555 nm; emission wavelength: 585 nm; cutoff 570 nm; autogain). Percentage death was determined by normalizing to water-treated cells and Raptinal-treated cells. For IC₅₀ determination, the data were plotted as compound concentration versus dead cell percentage and fitted to a logistic-dose-response curve using OriginPro 2019 (OriginLab). The data were generated in triplicate, and IC₅₀ values are reported as the average of three separate experiments along with SEM values.

Antibacterial bioassays. Susceptibility testing for the ESKAPE pathogens and Salmonella enterica LT2 was performed using a Kirby-Bauer method as outlined by the Clinical and Laboratory Standards Institute (CLSI). For rich media bioassays, Mueller Hinton Broth 2 (MH-2, Sigma-Aldrich, 90922) was used for all strains except 1% BHI was supplemented for Enterococcus faecalis ATCC 19433. For minimal media bioassays, glucose-MOPS minimal medium was used. Briefly, overnight bacterial cultures were sub-cultured and inoculated into 5 mL top agar (0.7% agar) for final concentration of 5×10⁵ CFU/mL. Then, 20 μL of a compound 2-fold dilution series (200, 100, 50, 25, 12.5, 6.25 μM in water) was spotted on 6 mm diameter blank paper discs (BD BBL™ 231039). Control discs received 20 μL of 50 mg/mL kanamycin. Plates were incubated at 35° C. for 20-24 hours. Minimum Inhibitory Concentration (MIC) was recorded as the lowest concentration of compound that resulted in a clear zone of inhibition around the disc. Sensitivity of the IPTG-inducible phosphonate uptake strain WM6242 to pantaphos was tested using a disk diffusion assay. Plates containing growth medium were overlayed with 5 mL of top agar (0.7% agar) inoculated with 100 μL (OD₆₀₀=0.8) of the phosphonate-specific E. coli indicator strain (WM6242) with or without addition of 1 mM IPTG. WM6242 is engineered with an IPTG-inducible, non-specific phosphonate uptake system (phnCDE). After the seeded overlay solidified, 6 mm paper disks were spotted with 10 μL of a dilution series (200, 100, 50, 25, 12.5, 6.25 μM in water) of pantaphos and applied to the plates that were then incubated at 37° C. for 24 hours. Phosphonate-specific activity was queried by comparing sensitivity to that of 200 μM kanamycin and 200 μM fosfomycin. Minimum Inhibitory Concentration (MIC) was recorded as the lowest concentration of compound that resulted in a clear zone of inhibition around the disc

Fungicidal bioassays. Methods for fungicide testing were adopted from the CLSI publication M27, Reference for Broth Dilution Antifungal. Briefly, a −80° C. fungal stock was streaked on the appropriate media as indicated previously and incubated at 35° C. for 24-48 hours. For Candida and Saccharomyces, a single colony was picked and resuspended in 1 mL 1×PBS and then diluted in growth medium to a final concentration of 1×10⁴ CFU/mL. For Aspergillus, spores from the hyphal growth on the growth medium plate were resuspended by swirling 1 mL of 90% saline+0.1% Tween-20 onto the lawn. This 1 mL yeast suspension was then diluted in growth medium to 1×10⁴ CFU/mL final concentration. Then, 2 μL of compound was added to 198 μL of the yeast suspension in a sterile 96-well round bottom plate. For positive controls, 2 μL of a stock solution of amphotericin B was added to a final concertation of 2 or 10 μM as specified. 200 μL of un-treated yeast suspension and an uninoculated media were included as controls. The plate was incubated at 35° C. for 24-48 hours depending on growth medium without shaking. Minimum Inhibitory Concentration (MIC) was determined visually by finding the concentration of compound at which there was no visual difference between that concentration and the uninoculated media control.

Identification and analysis of homologous Hvr biosynthetic gene clusters in bacteria. NCBI tblastn was used to identify bacterial genomes that contain Hvr-like gene clusters. The query was constructed by concatenating the gene translations of hvrA-L. Tblastn was used against the RefSeq Genomes and the RefSeq Representative Genomes databases using the ‘Organism’ parameter of ‘Bacteria [taxid:2]’, and the non-redundant nucleotide database. The results were filtered by 45% query coverage, which resulted in 185 unique bacterial strains. The GenBank files for each strain were downloaded and homologous Hvr gene clusters were determined using MultiGeneBlast. Homologous Hvr biosynthetic gene clusters were organized by type based on gene arrangement within the cluster and presence of additional gene functions.

### Example 2. Structure Elucidation of Phosphonate Compounds

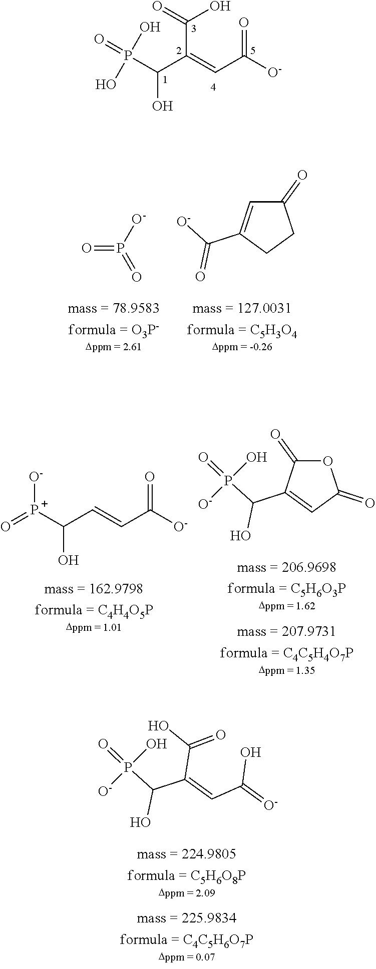

Structure elucidation of compound1: NMR spectral data are summarized in the main text (Table 1) and below; High-resolution mass-spectral data for compound 1 (purified pantaphos) are presented here. The designated mass fragments with assigned chemical structures are shown above. MS chemical formulas and mass error were calculated using ChemCalc workspace. Compound 1 was isolated as a white, amorphous solid. Its molecular formula was deduced by negative mode HIRMS (calcd. for C₅H₆O₈P⁻¹: 224.98058, observed m/z 224.9805 [Δppm 2.09]).

Compound 1 was dissolved in 100% D20 for NMR experiments. The ¹H-NMR spectrum for compound 1 revealed two signals at δ_{H} 4.31 and 5.91 ppm that appeared as doublets with coupling constants J of 15.3 Hz and 6.00 Hz, respectively. The large coupling constant J of 15.3 Hz is typical for protons bound to the adjacent carbon to a phosphorus atom in phosphonic acids. These protons were also correlated to the phosphorus atom of the compound at δ_{P} 15.40 ppm in the ¹H-³¹P HMBC analysis indicating close proximity (within 3-bond distance) to P. In addition, the downfield signal at δ_{H z}5.91 indicates a vinyl-carbon or alkene structure, which indicates that this signal corresponds to a single proton. The ¹³C-NMR spectrum revealed signals at δ_{C} 71.00 (d, J=144.00 Hz), 142.98 (s), 174.62 (s), 126.50 (d, J=9.05 Hz), and 175.20 (s) ppm indicating compound 1 contains five carbons. The large coupling constant of the signal at δ_{C} 71.00 ppm suggests this carbon is bonded to the phosphorus atom as this splitting pattern has been observed for C—P bonding in other phosphonic acid compounds. Therefore, this signal at δ_{C} 71.00 ppm is assigned as carbon position 1. None of the other carbon signals showed a typical C—P splitting pattern, therefore, the signals corresponding to these carbons must reflect carbon positions opposite the phosphonate moiety and adjacent to or nearby carbon 1.

Proton-carbon HSQC and HMBC experiments revealed the coupling of proton at δ_{H} 4.31 ppm to the carbon at position 1 (δ_{C} 71.00 ppm) and was observed to correlate to the other carbons at δ_{C} 142.98, 174.62, and 175.20 ppm supporting the assignment of these carbons at positions adjacent to or nearby carbon 1. The carbon signal at δ_{C} 142.98 ppm has no splitting pattern and aligns with the chemical shifts predicted for vinyl compounds bound to an adjacent carboxylic acid and methyl group suggesting carbon position 2 assignment. The similar carbon signals at δ_{C} 174.62 and 175.20 ppm have no splitting pattern and align with the chemical shifts predicted for carboxylic acids suggesting assignment to carbon positions 3 or 5. However, the splitting of the carbon signal at δ_{C} 126.50 ppm (d, J=9.05 Hz) indicates the presence of an adjacent proton as the ¹³C-NMR analysis was not performed with decoupling of ¹H. This is supported by the HSQC between this carbon and the proton at δ_{H} 5.91 ppm. These data fully support the assignment of the carbon at δ_{C} 126.50 ppm to carbon position 4. Based on the proton-carbon HMBC between the protons at δ_{H} 4.31 and 5.91 ppm and the carbons at δ_{C} 142.98, 174.62, and 175.20 ppm, we were able to confirm assignment of carbons at δ_{C} 142.98, 174.62, and 175.20 ppm to positions 2, 3, and 5, respectively. Finally, after ¹H-¹H correlation analyses, it was determined that the protons at δ_{H} 4.31 and 5.91 ppm are arranged in a cis carbon-carbon double bond configuration. Based on the agreements between the MS data and these NMR assignments the compound structure is identified as (E)-2-(hydroxy(phosphono)methyl)-4-oxopent-2-enoate.

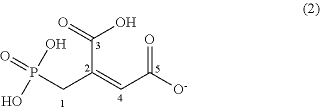 [SMILES: O=C([O-])/C=C(/CP(=O)(O)O)C(=O)O]

Structure elucidation of compound 2: NMR spectral data are summarized in the main text (Table 1); NMR spectra and high-resolution mass-spectral data for compound 2 are found at the end of this paragraph. MS chemical formulas and mass error were calculated using ChemCalc workspace. Compound 2 was isolated as a white, amorphous solid. Its molecular formula was deduced by negative mode HRMS (calcd. for C₅H₆O₇P⁻¹: 208.98566, observed m/z 208.9851 [Δppm −0.07]). Fractions containing pure compound 2 were dissolved in 100% D20 and subjected to proton and phosphorus NMR analyses. For carbon NMR experiments, a sample containing trace amounts of compound 1 were used as there was not enough concentrated pure compound 2 to perform carbon-13 analyses. The ¹H-NMR spectrum for compound 2 revealed two signals at δ 5.71 (d, J=6.00 Hz) and 2.43 ppm (d, J=18.0 Hz). The large coupling constant J of 18.0 Hz is typical for protons bound to the adjacent carbon to a phosphorus atom in phosphonic acids. These protons were also correlated to the phosphorus atom of the compound at r 18.46 ppm in the ¹H-³¹P HMBC analysis indicating close proximity (within 3-bond distance) to P. In addition, the downfield signal at δ_{H} 5.71 indicates a vinyl-carbon or alkene structure, which indicates that this signal corresponds to a single proton. ¹³C-NMR analysis revealed signals at δ_{C} 34.05 (d, J=123.0 Hz), δ 126.10 (d, J=10.60 Hz), δ 140.36 (s), δ 174.68 (s), and δ 177.02 (s) ppm associated with compound 2 indicating a five-carbon molecule. The large coupling constant of the carbon signal at δ_{C} 34.05 ppm suggests this carbon is bonded to the phosphorus atom as this splitting pattern has been observed for C—P bonding in other phosphonic acid compounds. Therefore, this signal at δ_{C} 34.05 ppm is assigned as carbon position 1. None of the other carbon signals showed a typical C—P splitting pattern, therefore, the signals corresponding to these carbons must reflect carbon positions opposite the phosphonate moiety and adjacent to or nearby carbon 1. Proton-carbon HSQC and HMBC experiments revealed the coupling of protons at δ_{H} 2.43 ppm to the carbon at position 1 (δ_{C} 34.05 ppm) and was observed to correlate to the other carbons at δ_{C} 140.36, 174.68, and 177.02 ppm supporting the assignment of these carbons at positions adjacent to or nearby carbon 1. The carbon signal at δ_{C} 140.36 ppm has no splitting pattern and aligns with the chemical shifts predicted for vinyl compounds bound to an adjacent carboxylic acid and methyl group suggesting carbon position 2 assignment. The similar carbon signals at δ_{C} 174.68 and 177.02 ppm have no splitting pattern and align with the chemical shifts predicted for carboxylic acids suggesting assignment to carbon positions 3 or 5. However, the splitting of the carbon signal at δ_{C} 126.10 ppm (d, J=10.60 Hz) indicates the presence of an adjacent proton as the ¹³C-NMR analysis was not performed with decoupling of ¹H. This is supported by the HSQC between this carbon and the proton at δ_{H} 5.71 ppm. These data fully support the assignment of the carbon at δ_{C} 126.10 ppm to carbon position 4. Based on the proton-carbon HMBC between the protons at δ_{H} 2.43 and 5.71 ppm and the carbons at δ_{C} 140.36, 174.68, and 177.02 ppm, we were able to confirm assignment of carbons at δ_{C} 140.36, 174.68, and 177.02 ppm to positions 2, 3, and 5, respectively. Finally, after ¹H-¹H correlation analyses, it was determined that the protons at δ_{H} 2.43 and 5.71 ppm are arranged in a cis carbon-carbon double bond configuration. Based on the agreements between the MS data and these NMR assignments the compound structure is identified as (E)-2-(phosphono)methyl)-4-oxopent-2-enoate.

### Example 3. Multigram Preparation of Phosphonate Compounds

Process flowchart for multigram scale preparation of phosphonate products disclosed herein:
1. Phosphate induction with IPTG in 10L bioreactor
2. Pellet cells and concentrate supernatant via freeze-drying
3. Methanol extraction of phosphonic acids
4. Purification from the methanol-soluble fraction using iron-IMAC
5. Further purification using flash chromatography and HILIC HPLC

### Detailed Steps of the Process:

Step 1: Frozen culture stock of phosphonate induction strain was streaked on LB medium and incubated ˜18 hours at 30° C.;

Step 2: Single colony transferred into 5 mL of phosphonate-induction-medium (PIM minus IPTG; see published reference for formulation) and incubated for 48 hours at 30° C.;

Step 3: 1 mL of previous culture transferred to 200 mL PIM (minus IPTG) and incubated 48 hours at 30° C.;

Step 4: 100 mL of previous culture was transferred to 10 Liters PIM (with 1 mM IPTG) in New Brunswick BIOFLO 110 Fermenter/Bioreactor and incubated for 96 hours as follows:
a) Oxygenation (bubbling air at bottom of reactor) through a sterile air filter with gauge set to ˜5 liters/min air flow rate;
b) Bioreactor equipped with heating jacket set to 30° C.;
c) Pantoea ananatis growth and phosphonate production indicated by yellow pigment development;

Step 5: Cells were pelleted (centrifugation 7500×g for 20 min) and culture supernatant (spent media) harvested and freeze dried until completely dehydrated;

Step 6: Phosphonic acids then extracted from the spent media using 75% cold methanol;

Step 7: The methanol-soluble fraction was separated either by filtration methods or centrifugation and the resulting solution was concentrated via rotary evaporation to remove methanol;

Step 8: methanol-soluble phosphonic acids were purified from the concentrated solution using 200 grams of Iron-IMAC resin affinity chromatography with a step-gradient of ammonium-bicarbonate up to 1 M. The resulting fractions were neutralized with acetic acid and concentrated using rotary evaporation;

Step 9: The resulting concentrated fractions containing phosphonic acids were further purified using a fast chromatography system with a strong-anion exchange column with a gradient of 5% ammonium-hydroxide. The resulting fractions that have an absorbance at 250 nm UV were pooled together and neutralized using acetic acid followed by concentration via rotary evaporation;

Step 10: The flash chromatography fractions can be further purified using HILIC HPLC with a gradient of acetonitrile+10 mM ammonium bicarbonate, pH 8.5 (solvent B) and water+10 mM ammonium bicarbonate, pH 8.5 (solvent A); fractions that absorb at 250 nm UV are collected and can be further separated using HILIC HPLC with a gradient of acetonitrile+10 mM ammonium acetate, pH 4 (solvent B) and water+10 mM ammonium acetate, pH 4 (solvent A). Finally, the remaining ammonium acetate is removed from pure compounds using HILIC HPLC with gradient of acetonitrile (solvent B) and water (solvent A).

### Example 4. Phosphonate-Related Gene Clusters Associated with Various Pathogenic and Non-Pathogenic P. Ananatis Strains

| | Isolation | Associated | Associated | | | Putative | Putative | | | # |
|---|---|---|---|---|---|---|---|---|---|---|
| Strain | host/ | with | with | | | phos- | phos | C-P | Genome | pepM |
| (sorted | environ- | plant | onion | Hvr | Pgb | phonate | phonate | lyase | completion | per |
| alphabetcally) | ment | [symbol] | disease | BGC * | BGC * | BGC-A * | BGC-B * | pathway ** | level | strain |
| P. ananatis 97-1 | onion | Y | Y | + | − | − | − | + | complete | 1 |
| P. ananatis AJ13355 | soil | n/a | nt | − | − | + | − | + | complete | 1 |
| P. ananatis AMG521 | rice | N^{g} | nt | − | − | − | − | + | scaffold | 0 |
| P. ananatis ARC272 | rice | n/a | nt | − | − | − | − | + | contig | 0 |
| P. ananatis ARC310 | rice | n/a | nt | − | − | − | − | + | contig | 0 |
| P. ananatis ARC311 | rice | n/a | nt | − | − | − | − | + | contig | 0 |
| P. ananatis (stewartii) | fungus | N | N | − | − | − | − | + | scaffold | 0 |
| B-133 *** | | | | | | | | | | |
| P. ananatis B-14773 *** | fungus | N | N | − | − | − | − | + | scaffold | 0 |
| P. ananatis B1-9 | onion | N^{g} | N | − | − | + | − | + | scaffold | 1 |
| P. ananatis B7 | maize | n/a | nt | + | − | − | − | + | contig | 1 |
| P. ananatis BAV 3296 | rain- | n/a | nt | − | − | − | − | + | contig | 0 |
| | isolated | | | | | | | | | |
| P. ananatisBD442 | maize | Y | N | − | − | − | + | + | contig | 1 |
| P. ananatis BRT175 | strawberry | n/a | nt | − | − | − | − | + | scaffold | 0 |
| P. ananatis CFH 7-1 | cotton | Y | nt | − | − | − | − | + | contig | 0 |
| P. ananatis DAR 76143 | rice | Y | nt | + | − | + | − | + | contig | 2 |
| P. ananatis DE0584 | soil | n/a | nt | − | − | − | − | + | scaffold | 0 |
| P. ananatis DZ-12 | maize | Y | nt | + | − | − | − | + | scaffold | 1 |
| P. ananatis F-C2 | acid mine | n/a | nt | + | − | − | − | + | contig | 1 |
| | drainage | | | | | | | | | |
| P. ananatis | clinical | n/a | nt | − | − | − | − | + | complete | 0 |
| FDAARGOS_680 | | | | | | | | | | |
| P. ananatis LMG 20103 | Eucalyptus | Y | Y | + | − | − | − | + | complete | 1 |
| P. ananatis LMG 2665 | pinneapple | Y | Y | + | − | − | − | + | scaffold | 1 |
| P. ananatis LMG 5342 | clinical | Y | Y | + | + | − | − | + | complete | 2 |
| P. ananatis M232A | maize | n/a | nt | + | − | − | − | + | contig | 1 |
| P. ananatis MMB-1 | soil | n/a | nt | + | + | − | − | + | contig | 2 |
| P. ananatis MR5 | groundnut | n/a ^{e} | nt | − | − | − | − | + | contig | 0 |
| | plant | | | | | | | | | |
| P. ananatis NFIX48 | n/a | n/a | nt | + | − | − | − | + | scaffold | 1 |
| P. ananatis NFR11 | n/a | n/a | nt | + | + | − | − | + | scaffold | 2 |
| P. ananatis NN08200 | sugarcane | n/a ^{e} | nt | − | − | − | − | + | complete | 0 |
| P. ananatis NS296 | rice seed | n/a ^{e} | nt | − | − | − | − | + | contig | 0 |
| P. ananatis NS303 | rice seed | n/a ^{e} | nt | − | − | − | − | + | contig | 0 |
| P. ananatis NS311 | rice seed | n/a ^{e} | nt | − | − | − | − | + | contig | 0 |
| P. ananatis PA13 | rice | Y | Y | + | − | + | − | + | complete | 2 |
| P. ananatisPA4 | onion seed | Y | Y | − | − | − | − | + | contig | 0 |
| P. ananatisPaMB1 | n/a | Y | nt | − | − | + | − | + | scaffold | 1 |
| P. ananatis PANS 01-2 | onion | Y | Y | + | − | − | − | + | contig | 1 |
| P. ananatis PANS 02-01 | n/a | n/a | nt | + | − | + | − | + | scaffold | 2 |
| P. ananatisPANS 04-2 | thrips | N | N | + | − | + | − | + | contig | 2 |
| P. ananatis PANS 200-1 | herb | N | N | − | − | − | − | + | scaffold | 0 |
| P. ananatis PANS 99-23 | grass | N | N | − | − | − | − | + | contig | 0 |
| P. ananatis PANS 99-3 | onion | Y | Y | + | − | − | − | + | contig | 1 |
| P. ananatis PANS 99-36 | coffee plant | N | N | − | − | − | − | + | contig | 0 |
| P. ananatis PNA 06-1 | onion | Y | Y | + | − | − | − | + | contig | 1 |
| P. ananatis PNA 07-1 | onion | Y | Y | + | − | − | − | + | scaffold | 1 |
| P. ananatisPNA 07-10 | onion | N | N | + | − | − | − | + | scaffold | 1 |
| P. ananatis PNA 11-1 | onion | N | N | − | − | − | − | + | scaffold | 0 |
| P. ananatisPNA 14-1 | onion | Y | Y | − | − | − | − | + | contig | 0 |
| P. ananatis PNA 15-1 | onion | Y | Y | + | − | − | − | + | contig | 1 |
| P. ananatisPNA 200-3 | onion seed | Y | Y | − | − | − | − | + | contig | 0 |
| P. ananatisPNA 200-7 | onion seed | N | N | + | − | − | − | + | scaffold | 1 |
| P. ananatis PNA 86-1 | soil | n/a | nt | − | − | − | − | + | scaffold | 0 |
| P. ananatis PNA 98-11 | onion | Y | Y | + | − | − | − | + | scaffold | 1 |
| P. ananatis PNA 99-7 | onion | N | N | − | − | − | − | + | contig | 0 |
| P. ananatis R100 | rice seed | n/a | nt | + | − | − | − | + | complete | 1 |
| P. ananatis RSA47 | rice seed | n/a ^{e} | n | − | − | − | − | + | contig | 0 |
| P. ananatis S6 | corn seed | n/a ^{g} | n | − | − | − | − | + | contig | 0 |
| P. ananatis S7 | corn seed | Y | nt | + | − | − | − | + | contig | 1 |
| P. ananatis S8 | corn seed | n/a ^{g} | nt | − | − | − | − | + | contig | 0 |
| P. ananatis Sd-1 | rice seed | n/a | nt | − | − | − | − | + | scaffold | 0 |
| P. ananatis SGAir0210 | tropical air | n/a | nt | − | − | − | − | + | chromo- | 0 |
| | | | | | | | | | some | |
| P. ananatis strain 1.38 | rice | N^{g} | nt | − | − | − | − | + | contig | 0 |
| P. ananatis SUPP2219 \hat{ } | rice | Y | Y | + | n/a | − | − | n/a | locus | 1 |
| P. ananatis UBA12293 | meta- | n/a | nt | − | − | − | − | + | scaffold | 0 |
| | genome | | | | | | | | | |
| P. ananatis | tomato and | Y | nt | − | − | − | − | + | contig | 0 |
| Weeds Lee_18a | pepper | | | | | | | | | |
| | phyllo- | | | | | | | | | |
| | sphere | | | | | | | | | |
| | meta- | | | | | | | | | |
| | genome | | | | | | | | | |
| P. ananatis YJ76 | rice | n/a ^{e} | nt | − | − | + | − | + | complete | 1 |
| Pantoea sp. AG702 | rice | n/a | nt | + | − | − | − | + | scaffold | 1 |

bold text = strains with exceptions to the correlation between hvr and pathogenicity
n/a = data not available
nt = not tested
^{g} = strains that show ability to promote plant growth based on data from the associated reference
^{e} = strains that exhibit an endophytic lifestyle based on data from associated reference
\hat{ } deposited as only a virulence determining genetic locus not full genome
* locus identified based on presence of pepM gene and the associated genomic region referenced to P. ananatis LMG 5342 hvr gene cluster
** locus identified based on presence of phnJ gene
*** denotes strains that were sequenced in this study (see Materials and Methods) [symbol]
indicates data missing or illegible when filed

| | | | Putative | Putative | |
|---|---|---|---|---|---|
| | | | phosphonate | phosphonate | C-P lyase |
| | Hvr BGC | Pgb BGC | BGC-A | BGC-B | pathway |
| Total positive strains | 27 | 3 | 8 | 1 | 64 |
| % of total strains | 42% | 5% | 12% | 2% | 98% |
| Total # strains with at | 32 | — | — | — | — |
| least 1 pepM | | | | | |
| % of strains with pepM | 49% | — | — | — | — |
| # strains with one pepM | 25 | — | — | — | — |
| # strains with two pepM | 7 | — | — | — | — |

### Example 5. Cytotoxicity Profiling

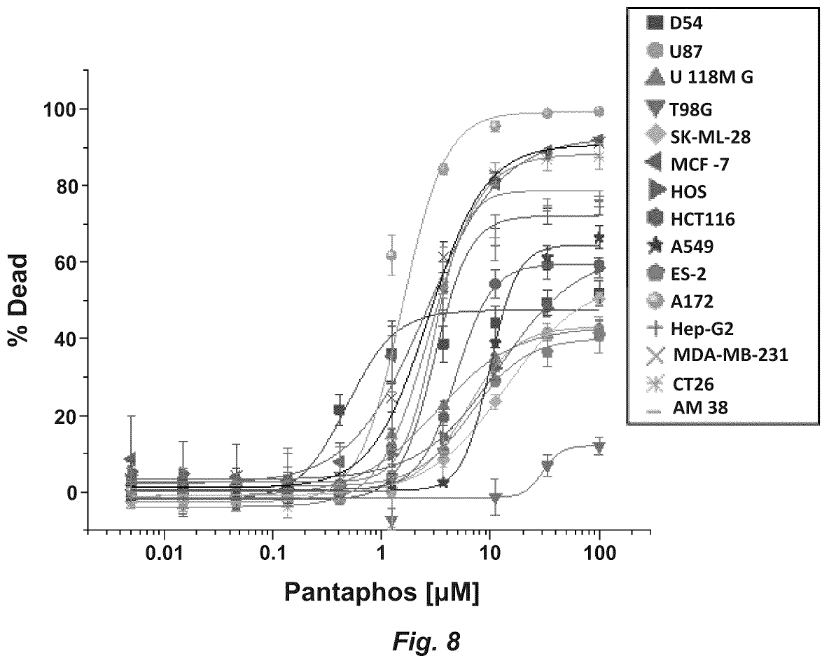

We previously conducted cytotoxicity profiling against a panel of human cell lines, including normal fibroblast cells (HFF-1 cell line), and five cancer cell lines (HOS (human osteosarcoma), ES-2 (human ovarian cancer), A-549 (human lung cancer) and A-172 (human glioma). Pantophos showed modest cytotoxicity to several human cell lines (Table 6). With the exception of one ovarian cancer cell line (ES-2), which was unaffected at the maximum dose, the IC₅₀ levels were roughly similar, in the range of 6.0 to 37.0 mM for each of the cell lines tested. One glioma cell line (A-172) was especially sensitive to pantaphos (IC₅₀ of 1.0 mM). To ask whether this sensitivity is specific to glioma cell lines, we expanded the panel to include an additional ten human cancer lines (FIG. 8 and Table 6). Consistent with previous data some cell lines are sensitive, while others are resistant, including the glioblastoma cell line TG98. Thus, pantaphos cytotoxicity is not a feature of all glioblastomas. However, cell lines from other cancer types, including MCF-7 (breast cancer) MDA-MB-231 (breast cancer) CT26 (colon carcinoma) HepG2 (liver cancer) AM38 (glioblastoma) were sensitive to the compound. These data suggest that these cell lines carry mutations that confer sensitivity to pantaphos, paving the way for use of the molecule as a therapy for carcinomas carrying these genetic markers.

TABLE 6
Cytotoxicity of pantaphos across various cancer cell lines.

| Cell line | IC₅₀ | Eₘₐₓ |
|---|---|---|
| D54 | ND | 46-58% |
| U87 | ND | 38-48% |
| U118MG | ND | 38-45% |
| T98G | >100 | ND |
| SK-ML-28 | ND | 47-51% |
| MCF-7 | 2.88 ± 6.28 | 90-92% |
| HOS | 36.98 ± 6.28 | 58% |
| HCT-116 | 10.42 ± 2.00 | 59% |
| A-549 | 14.73 ± 0.61 | 66% |
| ES-2 | >100 | ND |
| A-172 | 1.01 ± 0.06 | 99% |
| HepG2 | 4.22 ± 0.57 | 71-77% |
| MDA-MB-231 | 2.68 ± 0.23 | 91% |
| CT26 | 3.38 ± 0.09 | 81-92% |
| AM38 | 3.97 ± 1.01 | 74-78% |

### Example 6. Nucleic Acid and Amino Acid Sequences

| Gene | | SEQ ID |
|---|---|---|
| Name | Nucleic Acid Sequence | NO |
| hvrA | ATGATCAAAAAACTTATTGCAGAAAAGGGTACTCTGATTTTTATTGAGG | SEQ ID |
| | CCCATAATCCGCTCTCCGCATTAATTGCGTCTAAAGCAGAACAAACTAA | NO: 1 |
| | TTCAGAAGGCCGTATTGTCAAATTTGACGGTATATGGTCAAGCTCGTTA | |
| | ACGGACTCAGCATCTCGCGGTATTCCCGATAACGAAACACTGGCATTA | |
| | AGCAGCAGGTTAGAAAATATTGCTGATATCCGAAATGTGACAGACATG | |
| | CCCATCATCATGGATGCTGATACGGGGGGAAAACCAGAACATTTTAGT | |
| | TATTACGTAAAAAGAATGATTAACAACGGTGTAAATGGCGTCATCATC | |
| | GAAGATAAAACAGGATTAAAGAAAAATTCTTTGTTCGGCACTGAAGTA | |
| | GAACAGACTCTCGCAGATATTAATGATTTTTCAGAGAAGATTAAAAGA | |
| | GGAAAATCTGCAGTTTATATTGATGATTTTATGATCATAGCCAGACTTG | |
| | AAAGTCTTATTGCAGGGTTCGACGTAGAACATGCACTCGAACGTGCCG | |
| | ACGCATACGTCGAAGCCGGGGCAGACGGAATTATGATTCATAGTTGTA | |
| | AGAAGACTCCGGATGAGGTTTTCTTATTCAGTACGAAATTTCGGAAAA | |
| | AATATCCATCAGTACCATTAATTTGTGTTCCTACTACTTATTCTGCAACC | |
| | AGCAACAGAGAACTCAGTGAAGCGGGTTTTAACGTGATCATTTATGCA | |
| | AACCATATGCTCAGGGCTGCTTATAAAGCAATGGAAAATGTTTCAAAA | |
| | GAAATATTGAGATATGGCAGGACGGCAGAGATAGAAAAATCTTGCAT | |
| | GAGTGTAAAGGAAATTATTTCACTGATTCCTTAA | |
| hvrB | TTGCATGAGTGTAAAGGAAATTATTTCACTGATTCCTTAAAGAGAATGA | SEQ ID |
| | CCATGAAAAAAAAAGAGATGATAATAGGTGCCTATATATCGTATGGAA | NO: 2 |
| | CAGGACATCATCCTGCTTCATGGCGCGAAAGTGGCGTAAATGCAGCGG | |
| | CAGCGCTTGATATTGATACGTATGCCAATCTTGCAAGAGTATGTGAGA | |
| | AAGGATTAGCTGATTTACTTTTTCTTGCAGATACGCCTTCTGTATTTCAG | |
| | GACAATATGGACGGTTATGGCAGCAGGGTATCTGTACTGGAGCCATTA | |
| | TCATTATTATCGTATTTAGCATCTCAAACACAAAATATTGGGCTGGTTG | |
| | CGACGGCTTCAACGACGTACAAACACCCTTACAACATTGCGAGGGAAT | |
| | TTGCTTCGCTGGATTACATTAGCAAAGGAAGGGCAGGGTGGAATCTG | |
| | GTGACATCCTCAAAGTCGGATGCGGCTAAAAACTTTGGCCTTGCCGCT | |
| | CATCCAGAACATTCAAAGCGTTATGATATGGCCTGGGAAGCATGGCAG | |
| | GTTATCAGTGGTTTGTGGGACAGCTGGGAAGACAATGCTTTAGTAAGG | |
| | AATAAAACCAGTGGACAATTCTTCGTTAAGGATAAATACCGGGAAATA | |
| | AATTTTGAGGGTGAATATTTTAATGTAAAAGGCCCATTAAACATAGCTC | |
| | GCCCTCCTCAGGGCTATCCCGTCATCGTCCAGGCGGGTTCCTCTGAGG | |
| | AAGGAAAAGAGTTAGCTGCAAAAACAGCAGATATTGTTTTTACTGCAC | |
| | AAAACAATATTGAAGATGCAAAAAAATTCTATGATGATCTTAAAGGGC | |
| | GAATGGAAAAATACGGAAGGTCAAAGAGTGAACTTCTTATTCTTCCCG | |
| | GATTAAGCTTTTATATTGCAAGTGACGAATCTAAAGCCCGTAAAAAGCT | |
| | TAATGATCTCAACGCACTGATCCCTCAATCATTTGGCTTAAGTATGTTAT | |
| | CAGATTTACTGGGTGGGGTTGATTTAAAAAACAATGATCCTGAAGGAC | |
| | CATTACCAGATTTACCGAAATCTAATGGTAATCAGAGTAGACAAAAAA | |
| | TTATTATCGATTTAGCTCGAAAAGAAAAACTATCTATCAAGCAACTTTA | |
| | TGAAAAAATAATCATTTCAAGAGGACATTATACATTTACAGGTTCCTAT | |
| | CAAGATTTAGCAGATGAGATGATTAAGTGGGTTGAAAATGAAGCATGT | |
| | GATGGTTTCAACATTATGCCTCCTCTCATGCCTGAATCTCTTATTAATCT | |
| | TTTCGATCATGTCATTCCACTTATTCAGGCAAGAGGATGGTATAAAAAA | |
| | TCATATTCTACTGGAACATTAAGAGAAAAACTGGGGCTTAAAAGACCT | |
| | ACTAATAAATTGTTTAATCAATAA | |
| hvrC | ATGCTTAATAAAAATCTGATTCTTGAAGATACCACTTTACGTGATGGTG | SEQ ID |
| | AGCAGGCGCCAGGTGTTGCATTTACACCAGAGCAAAAAGTAGAAATTT | NO: 3 |
| | TTTATCTACTTGCAAATATGGGCGTTAAATGGATCGAAGCCGGAATAC | |
| | CTGCGATGAAGGGTGATGAAGTAAAGGCTCTGTCGGAAATGTTAGAG | |
| | AGAAAAAATGAAATTAACATCATCGCGTGGAACCGAGGCGTGCTTGAA | |
| | GACATTGAGTACAGTATCTCACTTGGATTCAAAGCGGTGCATATCGGG | |
| | CTACCGACTTCAGCTATCCATTTAGAGAAAAGCGTTAAGAAAGATAAG | |
| | TCCTGGCTTGTAAAGACGGCTTCAGATTTAGTTAAGTTTGCCAAAGACA | |
| | AAGGGATGTTTGTTTCTATCAGCGCAGAAGATATAGGCCGAACAGATA | |
| | TTGGATTCTTACAGGAGTATGCACAGGTAGTTGCTGAGGCTGGAGCCG | |
| | ATCGTCTTCGCCTCTCTGACACGATTGGTATTCTTTCTCCTGCACAATAC | |
| | AAAGAAAAAGTCTCTTTGTTAAATAAGAACGTCAACATCGATTTGCAGT | |
| | GCCATTGCCACAATGATTTTGGTTTTGCAGTTGCTAACACGCTGGCAGG | |
| | CATTGAAGCAGGAGCACGCTACTTTCATGTCTGCGTCAATGGCATTGG | |
| | TGAAAGGGCTGGAATGCCAGACCTGGCACAAGTTGCTATGGCATTGCA | |
| | CTTTTTCCACGGGGTTGATTTAGGGCTCGATTTAACAAAATTAATCGCG | |
| | TTGAGTGAAACGGTCGCCAGGTACAGCCATCAAAAAATCAGTCCATGG | |
| | CAGCCGATCGTAGGCGATAACGTTTTTGCACATGAATCGGGCATTCAC | |
| | GCAAATGGTATGCTCAAAGACAGCAGTACTTTTGAACCCTTCGACCCA | |
| | GCTACGGTGGGAGGAGAACGACGTCTGGTCGTGGGTAAACATTCCGG | |
| | TCGCGCCATTATCAAACATTTTCTCGAAGAATCAGGCGTGAAAGCTGCC | |
| | GACGATAAGGCTCTTGATCGCTGTTTAGAACGCGTGAGAAGTCATGCC | |
| | GTGCGCCACCCCGGTGGGATCCCTCCACATGTATTAGTTGATCTGTATA | |
| | CCGCGGGGTAA | |
| hvrD | ATGAAAAGTAATCAGCCGATTGTTAACCAGATCATTGCGTCACACAGT | SEQ ID |
| | GGCAGAGGTCAGGTTTCAGCAGGTGAACTGATCACGGTAGATGTTGA | NO: 4 |
| | CTACGTCTATGTTCAGGATGGAAATTCACCGACCGTGGCAAAACTGTTT | |
| | CAGGATTATCATCTGTCTGAGGTGCTAAAACCCGATAAAATCGGGTTCT | |
| | TCTTCGACCATTCAGTTCTGGTACCTGATAAAACCATGGCTAAACGTGT | |
| | CAACGAGGCCATGGAATTTGCAAAAAAACTTGGAATAAACATCTATTC | |
| | ACGAGGGGAGGGAATTAGTCACGTCATTGCCCTGGAGAGTAAAATAT | |
| | TTAAACCCGGCAATATAGTGCTGGGCGCAGATTCCCATACTTGTACAG | |
| | GGGGGGCCGTACAGTCTTTAGCGCTGGGAATGGGGGCTTCGGATATT | |
| | CTGGTTGCTATGTTAACAGGACAAACGTGGTTGAAAGTCCCTCAAACA | |
| | GTCCATTTGTGTATTAAGGGTAAAACGGGAAAAGATGTGCGGGCAAA | |
| | AGATGTCATGTTGGCACTTTTAAATAAGTACGGACAAACACCATTTCTT | |
| | TATAAATCGATCGAAGTTTCAGGGGAATGGGCAGAAGAGCTAACGCTT | |
| | GACGAAGCTGCAAGTTTTGCAAGTATGGCTGTTGAGTTAGGAGCCAAA | |
| | TGCATATTTATGCCAGATGGGCAAGGCAGGCCTGAGGGGCTATTGAA | |
| | GGCGGATGCCTCAGTGGCAGACAGTGTCATCAATTTTTCTGTATCAGA | |
| | ATTAATGCCCCATATCGCACCACCTCACAGTCCTTTGTATGCTAAACCTG | |
| | CAAATGACTTTGAGGGTCTGAAATTTGATTATATTTTCATTGGAAGCTG | |
| | TACTAACAGCAGACTTGAAGATATCAAAGAGGTGGCCGAAATTGTTGC | |
| | TGGTAAAACAATACATCCCGATATTCACTGCCTTCTGACGCCAGGATCG | |
| | AAAAGTGTTTATCTAAAAGCTCTCCAGGCGGGATATATCGATACGCTTA | |
| | TCCGCTCGGGCATTATTGTCACCCCACCGGGTTGTGGAGCTTGTGTGG | |
| | GTACCCAAGGAACCATTCCTGCGGATGGGGAGAAAGTATTAAGTACG | |
| | ATGAACCGCAATTTTAAGGGAAGAATGGGGAATGCTGAGGCAGACAT | |
| | CTTTTTATGTTCTCCACGAACTGCAGCGATGGTTGCATTGAACGGCACT | |
| | GTTCCACATTTTGAGGGAGAGTCCGCATATGAGTAA | |
| hvrE | CTGTTCCACATTTTGAGGGAGAGTCCGCATATGAGTAAAACTGCTGAA | SEQ ID |
| | AATTATCGCGTCAGACGCGTGGAAGGGAACATCTCGACCGACGATATT | NO: 5 |
| | ATACCTGCGCGCTATAAACATATGTATACCGAGCCGGCCCAGCTGGCA | |
| | CCGCATCTTTTTGAGAGCCGTTTTCCCGGATTTAGGGAAACGCTCAGTA | |
| | TCAATGATGTGCTTGTATGTGATCAAATATTCGGTATAGGGAGTTCGC | |
| | GGGAGCAGGCAGTAACAACTCTGTTGGCATGTGGTGTTAAATATGTAT | |
| | TTTCCCCTTCTTTCGGGAGGATTTTTTTTAGGAACTCTTGGAATTTGGGT | |
| | TTACATGCGATTGAGGTCGATACGAGTGAACTTGCAGATTTAAGTGAA | |
| | ATTAAAATAGAACTGACTGGAGGGGTAATTTATACAGAAAATAATCAA | |
| | ATAAATTTTTTCCCTCCCAGCTCGCAGATGACGGCAATTGTCAGTGCAG | |
| | GTGGCATAATACCCTACACCATAAATAAAATTATGGAAAAAAAAGGTG | |
| | ATATTTTAAGAGGTTATAGCAATGAAAAGTGA | |
| hvrF | ATGAAAAGTGAAAAGTTTGATGGTTTGGCTGATAACTATGATAAATAT | SEQ ID |
| | CGTCCCCGTTATCCTGCAATCCTTTTCAAGGAAATCCATGACTGGATGC | NO: 6 |
| | AGCCGTCTGCCAAAAATATATACGATATTGGCGCAGGCACAGGTATTG | |
| | CTATTGAAGGTATGACACGTGTCACTGGAAAACACTATGATTTCACGG | |
| | CGATAGATATTTCTGAAGATATGATAAAAAAAGGAAGGGAAAAACTG | |
| | CCTGGTACGACTTGGGTTAAAGGAAAAGCGGAAGATATTCTTTCTGAT | |
| | AAAAGCCGTATTGACGTCATTATGGCGGCACAGTCCTTCCAATGGATG | |
| | GATAGAGCTAAAACATTAGAAGTTTCGATAAAATCTTTAAATAAGGGC | |
| | GGGGTTTTTGCAGTTTTGCAAAACAATCGAGATTACAGAAATAATGAA | |
| | ATGCTTAACAAATATGAAGGTTTGCTAGAGAAATTTAGTCCAGGTTAC | |
| | AGCAGACATTATCGCGACTATGACTATGAAAATGAAATCACCAATGTTT | |
| | TTAAATTGCCTATTGCTAACTTTAAGAAAGTCGTCACAGGGTGGACTAT | |
| | GGAAATGATTTCAGAAGATTTTTTTGGATTCATTTCTTCCTCAACCCAG | |
| | GTACAAAGAGCTATTGAGAACGATCGTAATGGATTCTGGAAGGAGATT | |
| | GAAATCTTGATTGACGAACACTCAGTTGGTGGAAAAATTAGCATAGAT | |
| | TATATAAGTGAGTTATTTATAGCTAAGAAGCGTGATGATTCATAG | |
| hvrG | ATGATTCATAGCATGACGATTCAGGACATCAGCATTGAACAAGCTTCG | SEQ ID |
| | TATGAAGATGCGAAGCTGTTAAGAAAGGCTTTAGAAAAAGTTTACGAA | NO: 7 |
| | CCTTATACACTGAATTTCTCACCTACCGCTTTGCAGTTTACTGAAAATAT | |
| | CATTGCTCAGGAATCCTCGAAGTGGCTAGTCGCGAAATACAAGTCAGA | |
| | CATTGTGGGGGCAGTGAGATATGAACTTTATGATATTTATCTGGACTTC | |
| | CATTTTCTCTGCGTCACACCACCATTCAGAAAAATGGGAGTAGGGAAT | |
| | GAATTATTTCATAAACTGAAGAAAATAGCGTATGAAAAAAGAAAGGAT | |
| | TTTATGAAGATCGTTTTGCGAGATTCGCTAAGCTATAACCGACGCTATT | |
| | TTGAAAGTAAAGGATTTTACTTTTATCATAAATACCAGACAAATATGCA | |
| | TAGTGTATTTATTTTAAAATTAAACGGTGAAAAGCCATGA | |
| hvrH | ATGAATAAAAAAGCATTGGTTATTGGGTTGAAAAGTAATATGGAAAGA | SEQ ID |
| | GTCATCAAAGGGCTCAATGAGATAGAATTTATTATTATTGACAGAGGA | NO: 8 |
| | ACACTGGATAACGAAAGTATAGATTATATCATTAATCTTTCAGATGATT | |
| | TAATGCATAAAAAATCATTCAGTTATGTTATAGCCAGTTCTGAGGATTT | |
| | TATCGCTTTGGCTGGGTTATTGCGTAATCGTTATTCACTTTATGGTGAA | |
| | AAATATTATAAAAGTACAATTGCAACCAATAAATTCCTGATGCGTAATT | |
| | TTTGCTCAGGTTTTTTATCCTGTCCAAAGTTCTGGCTATCAGGTGAGAT | |
| | CATCAATTCAGAGAATCTTTTACTGTCATCCCAGAAAGATTACATCGTA | |
| | AAACCTCTTACAGGTAGTTCTGCAAAACATGTCGAAACAGTCACACAG | |
| | GAAGACTTAAACCAATATCTTAATGAAAATAACAAATTGATGTTAATTG | |
| | AAGAAAAGGTTTTAATGAGAGATGAATATCATCTTGATTGTATAATCA | |
| | AAGACGGTAATATCCTCTTTTCAACACTGTCAATTTACGACAGGCCTAT | |
| | ACTCGAGGCAAAAAGTAAAAACAGAGCCAGTATCAACTTACCCGATGG | |
| | CACACGGCTTCATGAAGATGCCTTAATACTTGCCACGAATTTGCAAAGT | |
| | CATTTTGAAATGACTAATGGCGTTTTTCATATTGAAATGTATCATACTCA | |
| | GGATGGATTTATACTCGGTGAGTTTGGTATCCGCCCTCCCGGAGCGGG | |
| | TGTGACCGATATGTATTATATGTATAGAGGCGTCGATTTCTGGGAGGC | |
| | ATTTATTTATTCTCAGATTGATAAAGAATTTATTTTACCCCTAAATCATA | |
| | AGTCCGATAAATATTGTGCTGCTATTGGGATATGTTCTTCTTTCCCGGT | |
| | CGATGATATCAGAAGTACGTCAAAAGTATCCGTGGATAAATATGTAAA | |
| | TTTGCGTGAGAATGCTGCTAAACCCGCAGTTCCCAGTTCAACGTCTTTT | |
| | AATCATATGATTTATGTTTCTTCATCATCCTTAGAAGAAATAAGGAATTT | |
| | TTTGCATGATATTTCTGACAGACAAGAGTAA | |
| hvrI | ATGTTAAAATATGAAACCAAAAAAAACGCTTTCTTCCAGATATACAATT | SEQ ID |
| | TTCTTTCATGCTCGGCTGAAGGGATTCAGACGGTTATATTTTTGTGGCT | NO: 9 |
| | TATATATCATGAAACTCATAGTCCGATGCTTGTCAGTCTGACTATAGTTT | |
| | CTTCATATCTTCCATCAGCCGTATTAGGATTCTTTTTCCTCAAAAAAGCT | |
| | GATGCGAGCAGTCCTGGAAAACAGTTATTTATCAGTAATGTTTCTCTTA | |
| | GTGCCATATCGCTAATTGTCTATTTTATTTTAATGAGAAATGAGGGTTTT | |
| | GAACTAATCACGCTCAGTATCTTTTATTTGGCACAGGCCGTATTATCTG | |
| | TTGTCAAAATGTTTAATAAGACATCCCAAAATCGCATTATCAGAACAGC | |
| | ATTCAGTAATAGTGATGCAATAAAAGCACTTCAGCTTGCCTCCTCTGGC | |
| | ATGCAGGCCGCTCAGGTTTTTGGCGCGGCCATAGGAGGATGGGCTATC | |
| | AGTACGGGTTACTACATGCAAGCGCTCATGCTGACCTGCTGCATCTACC | |
| | TTTTGAATATTTATATTTCGACACTTTTTGAGAAAGGGAATCCTGATAAT | |
| | AAAGACGCTGAGGTTATTGTGCAGATTCACTCTGTCACTAAGAAAAAT | |
| | GAATCCTTTACATATCTTTTTCGCAGTAAGGACTTTTTGTTACCCTTGAT | |
| | TTTTACTGTTCCATCAAGTGGAGCTTTACAATTTTTAAACACATCCCTTC | |
| | CTTCATTATCTTCTCTATATGGTAATAGCGAGAAAATTTATCCAGTGCTG | |
| | AATATGACTCTACAATGTGCTGTCATTATCTCGGGGATAGCAGCAGCA | |
| | CTCAATATGCTTTCTCTAAAGTCTTCATTACGGTTTTCTCTGTGTATTTCT | |
| | GGCATCTGTCTGATTTTGATGTGTTTATCATCGAGAAATTATTATGCCG | |
| | TATACTTTTTTTTATTTCTCACTTCTTTTTTTGTTTCATGGCATATGATTTC | |
| | TATTAAGGTACTCACGAATCAAATGCCTGATATAGAAAATATTGGTAAA | |
| | TTCACAATGATGAGAAACTCAGTCGCTTCTGGTGTGAAAATTGTCTTTT | |
| | CTATTTCGTCTGGTGCTTTTTTGACTTTTTACAGCATAACTACAACTTATC | |
| | TAATACTTGCGATACTGCTTATTTTTTTCAATGTACTATGGGTTTGTCAG | |
| | AGTCGCAGTTTTAACTATGAGGATTTAGGTGATGTTAAATTTCAAAAGT | |
| | GA | |
| hvrJ | ATGTTAAATTTCAAAAGTGATTTTTCTGAATTTATTACTGGTTTTTATCTT | SEQ ID |
| | AAACAGTTTACTCATTTAAATACGCAGGAAAGAGAACATGTGCTTGAA | NO: 10 |
| | ACGCTTGGCGTCACACCCAGCGCAATCAATGAGTTTATAACATCAGAA | |
| | GATATTTATATTACCTTACCTCATGCAAGTATGAATGTTTTTTTTCCAAG | |
| | AGCGGGGGTCTCACGTTATGTCTACGATCTACAAAATAGTAACAAAAA | |
| | CGCGTTCCATTTAAGGTTTTTCTTGACCCATACCAACTTTAGCGATCTGA | |
| | ACTGGCGCCCTTATGCATGGTGGTTTAATAACGGCGGGAAAATAGATA | |
| | AACTTACCTTTTTCACACGAAATAAAAAAAAGAAACATAATATCGTTTA | |
| | CTCGCTAAAACCAAATGAAATGCACTCTGCCTCTGTAGATCGACGTCTG | |
| | CGTCATGACTTTGATACAAGTATGAAGTTTAAACGTATTTCTCTTAGCTT | |
| | CATCTATATGACAGCCGTTCAAGAGGTCAATAGCGGTTTTGCACATAA | |
| | AGGCCGTACCCTTTATCTCCCACTTGACGCATTTGTAGCTTTTATTATTC | |
| | ATCAGGCAAAAAACGATGTTATATCTTTTAATTTTTTAGAAGCTTTTTTA | |
| | AGCCAGGCACAATGCAGAAGACTGAATGGCGAAGAATTGAGTTTTACC | |
| | AGTGAATGGCGCGATGCATTTATCTTTGATAATTTTACAAACATTGCGC | |
| | TGTTAAATTTTTTTCAGCCTGCAGCATTTGTGGGAGGCGAAAAAATGG | |
| | ATAACTACTGGCATCAGGTGATTGAAAAATGGAAAATGGCATTACCAA | |
| | ATCAATCTGAAGTTGAGTTTGCGTTACCCACTAATCTGGTCATGCCTGC | |
| | CGTTCAAGAGTATATTTACCCTTATAAACCATCAAGCGATATAGCAGAA | |
| | CAGCTTATTAAAAATAACATCCCATACAGTCTTACCATGGCTATTCAGG | |
| | AACATGATCTTTTTTCAAAGAAAGAATAA | |
| hvrK | ATGCTAGATAAAAGTGCTTTTCGCCACGCAATGTCGCATCTTCCTACTG | SEQ ID |
| | CAGTCACTATTGTAACATCAAGCGGCTCCTGCGGGGCTGCAGCATGTA | NO: 11 |
| | CAGTTTCATCCGTGTGTTCTGTCACCGACGACCCACCCACTTTACTCGTA | |
| | TGTATAAATCGTGCCTCCAATAACAATAGTGTTATAAGAAATAATGGCT | |
| | CTTTGTGTGTCAGCATTCTTTCAGGAGAGCAAAGCAACATTGCAATGCA | |
| | ATGTGCTAATCATCAGGTTTCAGTTGAGGAGCGCTTAGCATTATTCGAC | |
| | GCAGACGTTCTGGTCACGGGGTCTCCTGCTGTAGTTGATTCCGTTTGCT | |
| | GTTTAGATTGTCGTGTAGATAATATTGTGGAATCAGGAACCCACTCTGT | |
| | CTTCTTTTGTCAGGTTCTTTCTTCTAAAGCTTTTGTGGGGAAAGATGCG | |
| | CTGGTTTATTATTGCCGGAATTATCATCGTGTATCATAA | |
| hvrL | ATGATGAAAAGTTTAATTATTACGTCGCATTTTAATAGTGATTTATCCTC | SEQ ID |
| | TATGGCATGCGATGAGTTTGGATTAAAAAGATCACTAATTTTCTATACT | NO: 12 |
| | CCAAAAATAAATGAACTTAGCTTAGGTTTAAAATATGAGTACTACGATA | |
| | AGATAGATTCTTACTCTGTAAGATTAATGGAGCTTGGGAATGAAATTG | |
| | ATTTTATATGGAGCACTTCCGGTTCTGAAATCAACTCGTTCGCTGAATT | |
| | AAAAGCTGTTGAGTTGGCAGGATTAAGAAACATCTATCTAGGCATGAA | |
| | TGAAAATGCTTTTTCTACAGCTGCTTATAAAAGCATCACGCAAGAGTTA | |
| | ATGGCATTTTTGGGTGTTAGTATTCCCAAAGGGCTTCAATGTAACACAA | |
| | AAAAAGAAATACATGATTTCCTTGAATATAATGGTGGAACTATAGTCT | |
| | GTAAAGCTAACAATGGAGCAGGTGGTGTAAATCAATTTTATTGCCAAA | |
| | GATTTGATGACATATATAAACTCCCTCATGAGGTAACTGATTGGTATGT | |
| | AGAACAGTTTCTGAAAGGACTTGAGTTTTCTGTAAACGCTTATATGCTA | |
| | AATGGGTTTTATATAGCCTCCCCGATAATGTTTAAAGGCGAAACTGATA | |
| | TTCATAGCGGGCACGCAATGGACAAATTCAGGTATATTAGCAAGCTAA | |
| | AAAATAAAAGCTTAAATGAAAAAATAAATGCCATATTAAGTAAAATTA | |
| | GTAATACCAATATCTTTAATGGATGGATAGAGGTTGAGTTTATAAAAA | |
| | CACATCAGGATTTGGTAGTTATAGAAATTAATGCGAGGTATAATGGAA | |
| | CAATCAGAGCTACTGGATATGCTTGTAATGAAAATTTATATCAATTGGA | |
| | TTTGGAAAGTAAAATTTATAATAAATTTTCTTCACAATTAAATCATGAAA | |
| | ACGAAGTCATTGAGATGCCAATTCATTTGAAACTTGAAACAGGTTTAA | |
| | AAGAATTTGGTTTCGTCCAAAAAATGAAATCTCGAAAAACCAACACAG | |
| | GAAGGGCCACAATATGGGGTGAAGATCAATGTGAACTATTAGAGAGG | |
| | ATCAAAAATACTGAATTAGAAATATACTCAGAAAGAATTATTCATGGCA | |
| | TAAATGAGAGTAAGGAGTTATTCGAAAAATATATTTAA | |
| pTAC | GCGCAACGCAATTAATGTGAGTTAGCTCACTCATTAGGCACCCCAGGC | SEQ ID |
| promotor | TTTACACTTTATGCTTCCGGGTTTTCTTTGCCTCACGATCGCCCCCAAAA | NO: 13 |
| sequence | CACATAACCAATTGTATTTATTGAAAAATAAATAGATACAACTCACTAA | |
| | ACATAGCAATTCAGATCCGGTCCAGTAATGACCTCAGAACTCCATCTGG | |
| | ATTTGTTCAGAACGCTCGGTTGCCGCCGGGCGTTTTTTATTGGTGAGAA | |
| | TCGCAGCAACTTGTCGCGCCAATCGAGCCATGTCGTCGTCAACGACCC | |
| | CCCATTCAAGAACAGCAAGCAGCATTGAGAACTTTGGAATCCAGTCCC | |
| | TCTTCCACCTGCTGATCCGGATCGATCCCGTTATCGACTGCACGGTGCA | |
| | CCAATGCTTCTGGCGTCAGGCAGCCATCGGAAGCTGTGGTATGGCTGT | |
| | GCAGGTCGTAAATCACTGCATAATTCGTGTCGCTCAAGGCGCACTCCC | |
| | GTTCTGGATAATGTTTTTTGCGCCGACATCATAACGGTTCTGGCAAATA | |
| | TTCTGAAATGAGCTGTTGACAATTAATCATCGGCTCGTATAATGTGTGG | |
| | AATTGTGAGCGGATAACAATTTCACACAGGAAACAGAATTGATCCGGC | |
| | CAAGCTTGTAAGAAGGAGATATACAT | |
| Protein | | SEQ ID |
| Name | Amino Acid Sequence | NO |
| HvrA | MIKKLIAEKGTLIFIEAHNPLSALIASKAEQTNSEGRIVKFDGIWSSSLTDSAS | SEQ ID |
| | RGIPDNETLALSSRLENIADIRNVTDMPIIMDADTGGKPEHFSYYVKRMIN | NO: 14 |
| | NGVNGVIIEDKTGLKKNSLFGTEVEQTLADINDFSEKIKRGKSAVYIDDFMII | |
| | ARLESLIAGFDVEHALERADAYVEAGADGIMIHSCKKTPDEVFLFSTKFRKK | |
| | YPSVPLICVPTTYSATSNRELSEAGFNVIIYANHMLRAAYKAMENVSKEILR | |
| | YGRTAEIEKSCMSVKEIISLIP | |
| Hvrb | MHECKGNYFTDSLKRMTMKKKEMIIGAYISYGTGHHPASWRESGVNAA | SEQ ID |
| | AALDIDTYANLARVCEKGLADLLFLADTPSVFQDNMDGYGSRVSVLEPLSL | NO: 15 |
| | LSYLASQTQNIGLVATASTTYKHPYNIAREFASLDYISKGRAGWNLVTSSKS | |
| | DAAKNFGLAAHPEHSKRYDMAWEAWQVISGLWDSWEDNALVRNKTSG | |
| | QFFVKDKYREINFEGEYFNVKGPLNIARPPQGYPVIVQAGSSEEGKELAAK | |
| | TADIVFTAQNNIEDAKKFYDDLKGRMEKYGRSKSELLILPGLSFYIASDESKA | |
| | RKKLNDLNALIPQSFGLSMLSDLLGGVDLKNNDPEGPLPDLPKSNGNQSR | |
| | QKIIIDLARKEKLSIKOLYEKIIISRGHYTFTGSYQDLADEMIKWVENEACDG | |
| | FNIMPPLMPESLINLFDHVIPLIQARGWYKKSYSTGTLREKLGLKRPTNKLF | |
| | NQ | |
| HvrC | MLNKNLILEDTTLRDGEQAPGVAFTPEQKVEIFYLLANMGVKWIEAGIPA | SEQ ID |
| | MKGDEVKALSEMLERKNEINIIAWNRGVLEDIEYSISLGFKAVHIGLPTSAI | NO: 16 |
| | HLEKSVKKDKSWLVKTASDLVKFAKDKGMFVSISAEDIGRTDIGFLQEYAQ | |
| | VVAEAGADRLRLSDTIGILSPAQYKEKVSLLNKNVNIDLQCHCHNDFGFAV | |
| | ANTLAGIEAGARYFHVCVNGIGERAGMPDLAQVAMALHFFHGVDLGLDL | |
| | TKLIALSETVARYSHQKISPWQPIVGDNVFAHESGIHANGMLKDSSTFEPF | |
| | DPATVGGERRLVVGKHSGRAIIKHFLEESGVKAADDKALDRCLERVRSHAV | |
| | RHPGGIPPHVLVDLYTAG | |
| HvrD | MKSNQPIVNQIIASHSGRGQVSAGELITVDVDYVYVQDGNSPTVAKLFQD | SEQ ID |
| | YHLSEVLKPDKIGFFFDHSVLVPDKTMAKRVNEAMEFAKKLGINIYSRGEGI | NO: 17 |
| | SHVIALESKIFKPGNIVLGADSHTCTGGAVQSLALGMGASDILVAMLTGQT | |
| | WLKVPQTVHLCIKGKTGKDVRAKDVMLALLNKYGQTPFLYKSIEVSGEWA | |
| | EELTLDEAASFASMAVELGAKCIFMPDGQGRPEGLLKADASVADSVINFS | |
| | VSELMPHIAPPHSPLYAKPANDFEGLKFDYIFIGSCTNSRLEDIKEVAEIVAG | |
| | KTIHPDIHCLLTPGSKSVYLKALQAGYIDTLIRSGIIVTPPGCGACVGTQGTIP | |
| | ADGEKVLSTMNRNFKGRMGNAEADIFLCSPRTAAMVALNGTVPHFEGE | |
| | SAYE | |
| HvrE | MFHILRESPHMSKTAENYRVRRVEGNISTDDIIPARYKHMYTEPAQLAPHL | SEQ ID |
| | FESRFPGFRETLSINDVLVCDQIFGIGSSREQAVTTLLACGVKYVFSPSFGRI | NO: 18 |
| | FFRNSWNLGLHAIEVDTSELADLSEIKIELTGGVIYTENNQINFFPPSSQMT | |
| | AIVSAGGIIPYTINKIMEKKGDILRGYSNEK | |
| HvrF | MKSEKFDGLADNYDKYRPRYPAILFKEIHDWMQPSAKNIYDIGAGTGIAIE | SEQ ID |
| | GMTRVTGKHYDFTAIDISEDMIKKGREKLPGTTWVKGKAEDILSDKSRIDV | NO: 19 |
| | IMAAQSFQWMDRAKTLEVSIKSLNKGGVFAVLQNNRDYRNNEMLNKYE | |
| | GLLEKFSPGYSRHYRDYDYENEITNVFKLPIANFKKVVTGWTMEMISEDFF | |
| | GFISSSTQVQRAIENDRNGFWKEIEILIDEHSVGGKISIDYISELFIAKKRDDS | |
| HvrG | MIHSMTIQDISIEQASYEDAKLLRKALEKVYEPYTLNFSPTALQFTENIIAQE | SEQ ID |
| | SSKWLVAKYKSDIVGAVRYELYDIYLDFHFLCVTPPFRKMGVGNELFHKLK | NO: 20 |
| | KIAYEKRKDFMKIVLRDSLSYNRRYFESKGFYFYHKYQTNMHSVFILKLNGE | |
| | KP | |
| HvrH | MNKKALVIGLKSNMERVIKGLNEIEFIIIDRGTLDNESIDYIINLSDDLMHKK | SEQ ID |
| | SFSYVIASSEDFIALAGLLRNRYSLYGEKYYKSTIATNKFLMRNFCSGFLSCPK | NO: 21 |
| | FWLSGEIINSENLLLSSQKDYIVKPLTGSSAKHVETVTQEDLNQYLNENNKL | |
| | MLIEEKVLMRDEYHLDCIIKDGNILFSTLSIYDRPILEAKSKNRASINLPDGTR | |
| | LHEDALILATNLQSHFEMTNGVFHIEMYHTQDGFILGEFGIRPPGAGVTD | |
| | MYYMYRGVDFWEAFIYSQIDKEFILPLNHKSDKYCAAIGICSSFPVDDIRST | |
| | SKVSVDKYVNLRENAAKPAVPSSTSFNHMIYVSSSSLEEIRNFLHDISDRQE | |
| HvrI | MLKYETKKNAFFQIYNFLSCSAEGIQTVIFLWLIYHETHSPMLVSLTIVSSYL | SEQ ID |
| | PSAVLGFFFLKKADASSPGKQLFISNVSLSAISLIVYFILMRNEGFELITLSIFYL | NO: 22 |
| | AQAVLSVVKMFNKTSQNRIIRTAFSNSDAIKALQLASSGMQAAQVFGAAI | |
| | GGWAISTGYYMQALMLTCCIYLLNIYISTLFEKGNPDNKDAEVIVQIHSVTK | |
| | KNESFTYLFRSKDFLLPLIFTVPSSGALQFLNTSLPSLSSLYGNSEKIYPVLNM | |
| | TLQCAVIISGIAAALNMLSLKSSLRFSLCISGICLILMCLSSRNYYAVYFFLFLT | |
| | SFFVSWHMISIKVLTNQMPDIENIGKFTMMRNSVASGVKIVFSISSGAFLT | |
| | FYSITTTYLILAILLIFFNVLWVCQSRSFNYEDLGDVKFQK | |
| HvrJ | MLNFKSDFSEFITGFYLKQFTHLNTQEREHVLETLGVTPSAINEFITSEDIYIT | SEQ ID |
| | LPHASMNVFFPRAGVSRYVYDLQNSNKNAFHLRFFLTHTNFSDLNWRPY | NO: 23 |
| | AWWFNNGGKIDKLTFFTRNKKKKHNIVYSLKPNEMHSASVDRRLRHDFD | |
| | TSMKFKRISLSFIYMTAVQEVNSGFAHKGRTLYLPLDAFVAFIIHQAKNDVI | |
| | SFNFLEAFLSQAQCRRLNGEELSFTSEWRDAFIFDNFTNIALLNFFQPAAFV | |
| | GGEKMDNYWHQVIEKWKMALPNQSEVEFALPTNLVMPAVQEYIYPYKP | |
| | SSDIAEQLIKNNIPYSLTMAIQEHDLFSKKE | |
| HvrK | MLDKSAFRHAMSHLPTAVTIVTSSGSCGAAACTVSSVCSVTDDPPTLLVCI | SEQ ID |
| | NRASNNNSVIRNNGSLCVSILSGEQSNIAMQCANHQVSVEERLALFDADV | NO: 24 |
| | LVTGSPAVVDSVCCLDCRVDNIVESGTHSVFFCQVLSSKAFVGKDALVYYC | |
| | RNYHRVS | |
| HvrL | MMKSLIITSHFNSDLSSMACDEFGLKRSLIFYTPKINELSLGLKYEYYDKIDSY | SEQ ID |
| | SVRLMELGNEIDFIWSTSGSEINSFAELKAVELAGLRNIYLGMNENAFSTA | NO: 25 |
| | AYKSITQELMAFLGVSIPKGLQCNTKKEIHDFLEYNGGTIVCKANNGAGGV | |
| | NQFYCQRFDDIYKLPHEVTDWYVEQFLKGLEFSVNAYMLNGFYIASPIMF | |
| | KGETDIHSGHAMDKFRYISKLKNKSLNEKINAILSKISNTNIFNGWIEVEFIK | |
| | THQDLVVIEINARYNGTIRATGYACNENLYQLDLESKIYNKFSSQLNHENEV | |
| | IEMPIHLKLETGLKEFGFVQKMKSRKTNTGRATIWGEDQCELLERIKNTELE | |
| | IYSERIIHGINESKELFEKYI | |

### Example 7. Pharmaceutical Dosage Forms

The following formulations illustrate representative pharmaceutical dosage forms that may be used for the therapeutic or prophylactic administration of a composition of a formula described herein, a composition specifically disclosed herein, or a pharmaceutically acceptable salt or solvate thereof (hereinafter referred to as ‘Composition X’):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/mL) | mg/mL |
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | mg/mL |
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |
| (vi) Aerosol | mg/can |
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |
| (vii) Topical Gel 1 | wt. % |
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | wt. % |
| ‘Composition X’ | 5% |
| Methylcellulose | 2% |
| Methyl paraben | 0.2% |
| Propyl paraben | 0.02% |
| Purified water | q.s. to 100 g |
| (ix) Topical Ointment | wt. % |
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | wt. % |
| ‘Composition X’ | 5% |
| White bees wax | 10% |
| Liquid paraffin | 30% |
| Benzyl alcohol | 5% |
| Purified water | q.s. to 100 g |
| (xi) Topical Cream 2 | wt. % |
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1-20. (canceled)

21. A compound of Formula I: 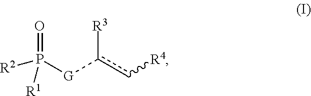
or a salt thereof;
wherein [symbol]
represents single or double bond; [symbol]
represents double or single bond, wherein both [symbol] and [symbol] are not double bonds;
G is X^{A}CHOR⁵, O, C(═O), C(═CH₂), CHP(═O)(R⁶)₂, or CX^{B}₂;
X^{A} is absent or O;
each X^{B} is independently H or halo;
R¹ and R² are each independently OR^{A} or an amino acid;
R³ is —C(═O)R⁷ or a triazole or tetrazole;
R⁴ is —C(═O)R⁸ or a triazole or tetrazole;
R⁵ is H, —(C₁-C₆)alkyl, —(C₃-C₆)cycloalkyl, aryl, or heteroaryl;
each R⁶ is independently OR^{B} or an amino acid;
R⁷ and R⁸ are each independently OR^{C} or an amino acid; and
each R^{A}, R^{B} and R^{C} are independently H, —(C₁-C₆)alkyl, —(C₃-C₆)cycloalkyl, aryl, or heteroaryl;
wherein the compound is not 2-(hydroxy(phosphono)methyl)maleic acid or 2-(phosphonomethyl)maleic acid (pantaphos).

22. A nucleic acid molecule comprising hvr operon of Pantoea Sp. and optionally an inducible promoter operably linked to the hvr operon.

23. The nucleic acid molecule of claim 22, comprising one or more genes selected from the group consisting of hvrA, hvrB, hvrC, hvrD, hvrE, and hvrK, wherein the one or more genes are operably linked to the inducible promoter.

24. The nucleic acid molecule of claim 22, comprising the inducible promoter according to SEQ ID NO: 13 and a nucleic acid sequence according to SEQ ID NO: 1, 2, 3, 4, 5, and 11 encoding genes hvrA, hvrB, hvrC, hvrD, hvrE, and hvrK, respectively, wherein the genes are operably linked to the inducible promoter.

25. The nucleic acid molecule of claim 22, wherein the Pantoea Sp. is Pantoea ananatis.

26. The nucleic acid molecule of claim 22, wherein the inducible promoter is a tac promoter.

27. The nucleic acid molecule of claim 22, comprising one or more genes selected from the group consisting of hvrA, hvrB, hvrC, hvrD, hvrE, and hvrK, wherein the one or more genes are operably linked to the inducible promoter.

28. An expression vector comprising the nucleic acid molecule of claim 22, wherein induction of the promoter and expression of the genes causes the production of a phosphonate compound of Formula I: 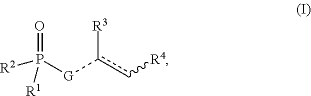
or a salt thereof;
wherein [symbol]
represents single or double bond; [symbol]
represents double or single bond, wherein both [symbol] and [symbol] are not double bonds;
G is X^{A}CHOR^{5′} O, C(═O), C(═CH₂), CHP(═O)(R⁶)₂, or CX^{B}₂;
X^{A} is absent or O;
each X^{B} is independently H or halo;
R¹ and R² are each independently OR^{A} or an amino acid;
R³ is —C(═O)R⁷ or a triazole or tetrazole;
R⁴ is —C(═O)R⁸ or a triazole or tetrazole;
R⁵ is H, —(C₁-C₆)alkyl, —(C₃-C₆)cycloalkyl, aryl, or heteroaryl;
each R⁶ is independently OR^{B} or an amino acid;
R⁷ and R⁸ are each independently OR^{C} or an amino acid; and
each R^{A}, R^{B} and R^{C} are independently H, —(C₁-C₆)alkyl, —(C₃-C₆)cycloalkyl, aryl, or heteroaryl.

29. The expression vector of claim 28, wherein the phosphonate compound is 2-(hydroxy(phosphono)methyl)maleic acid (pantaphos).

30. A recombinant cell for producing a phosphonate compound comprising the nucleic acid molecule of claim 22, wherein the phosphate compound is represented by Formula I: 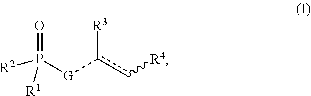
or a salt thereof;
wherein [symbol]
represents single or double bond; [symbol]
represents double or single bond, wherein both [symbol] and [symbol] are not double bonds;
G is X^{A}CHOR⁵, O, C(═O), C(═CH₂), CHP(═O)(R⁶)₂, or CX^{B}₂;
X^{A} is absent or O;
each X^{B} is independently H or halo;
R¹ and R² are each independently OR^{A} or an amino acid;
R³ is —C(═O)R⁷ or a triazole or tetrazole;
R⁴ is —C(═O)R⁸ or a triazole or tetrazole;
R⁵ is H, —(C₁-C₆)alkyl, —(C₃-C₆)cycloalkyl, aryl, or heteroaryl;
each R⁶ is independently OR^{B} or an amino acid;
R⁷ and R⁸ are each independently OR^{C} or an amino acid; and
each R^{A}, R^{B} and R^{C} are independently H, —(C₁-C₆)alkyl, —(C₃-C₆)cycloalkyl, aryl, or heteroaryl.

31. The recombinant cell of claim 30, wherein the nucleic acid molecule is integrated into a chromosome of the cell.

32. The recombinant cell of claim 30, wherein the cell is selected from a genus from the group consisting of Pantoea, Clostridium, Zymomonas, Escherichia, Salmonella, Serratia, Erwinia, Klebsiella, Shigella, Rhodococcus, Pseudomonas, Bacillus, Lactobacillus, Lactococcus, Enterococcus, Alcaligenes, Paenibacillus, Arthrobacter, Corynebacterium, Brevibacterium, Schizosaccharomyces, Kluveromyces, Yarrowia, Pichia, Zygosaccharomyces, Debaryomyces, Candida, Brettanomyces, Pachysolen, Hansenula, Issatchenkia, Trichosporon, Yamadazyma, and Saccharomyces.

33. The recombinant cell of claim 30, wherein the cell is of the genus Pantoea, Escherichia, or Saccharomyces; or wherein the cell is Pantoea ananatis, Escherichia coli, or Saccharomyces cerevisiae.

34. A process for producing a phosphonate compound according to Formula I comprising the steps of:
a) providing a cell culture of a recombinant cell of claim 30, wherein the recombinant cell produces the phosphonate, and the cell culture is about 1 L to about 10 L in volume;
b) mixing an inducer molecule with the cell culture;
c) incubating the induced cell culture for up to 96 hours with constant oxygenation;
d) pelleting cells of the cell culture and collecting a supernatant;
e) concentrating the supernatant;
f) extracting the phosphonate from the concentrated supernatant using methanol extraction to form an extracted supernatant; and
g) purifying the phosphonate from a methanol soluble fraction of the extracted supernatant.

35. The process of claim 34, wherein the phosphonate is 2-(hydroxy(phosphono)methyl)maleic acid.

36. The process of claim 34, wherein step g comprises iron-IMAC purification followed by flash chromatography and HILIC HPLC.

37. The process of claim 34, wherein the cell culture comprises Pantoea ananatis, Escherichia coli, or Saccharomyces cerevisiae; or wherein the cell culture is Pantoea ananatis.

38. The process of claim 34, wherein the cell culture is Pantoea ananatis, the constant oxygenation has a flow rate of 5 L/min, and the cell culture is maintained at a temperature of 30° C.

39. A method for forming 2-(hydroxy(phosphono)methyl)maleic acid: 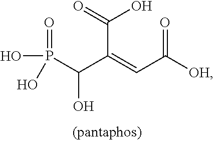 [SMILES: O=C(O)/C=C(\C(=O)O)C(O)P(=O)(O)O]
or salt thereof, comprising:
a) isomerizing phosphoenolpyruvate (PEP) to 3-phosphonopyruvate (PnPy);
b) condensing an acetyl group and PnPy to form phosphonomethylmalate (PMM);
c) dehydrating PMM to 2-phosphonomethylmaleate; and
d) oxidizing 2-phosphonomethylmaleate to pantaphos;
wherein each step a)-d) is completed in a vessel.

40. The method of claim 39, wherein isomerizing is catalyzed by PEP mutase (HvrA); condensing is catalyzed by phosphonomethylmalate synthase (HvrC) and the acetyl group is acetyl-CoA; dehydrating is catalyzed by large isopropylmalate dehydratase (HvrD) and/or small isopropylmalate dehydratase (HvrE) dehydratase; and oxidizing is catalyzed by flavin-dependent monooxygenase (HvrB) and optionally flavin reductase (HvrK).
